(19) United States
(12) Patent Application Publication
(10) Pub. No.: US 2025/0127804 A1
(43) Pub. Date: Apr. 24, 2025

(54) RIBOSE LINKERS AND CONJUGATES THEREOF

(71) Applicants: ELYSIUM HEALTH INC., New York, NY (US); UNIVERSITY OF SOUTH ALABAMA, Mobile, AL (US)

(72) Inventors: Ryan DELLINGER, New York, NY (US); Marie MIGAUD, Mobile, AL (US); Jyothi DHUGURU, Mobile, AL (US)

(21) Appl. No.: 18/833,971

(22) PCT Filed: Feb. 3, 2023
(86) PCT No.: PCT/US2023/061917

§ 371 Date: Jul. 29, 2024

Related U.S. Application Data
(60) Provisional application No. 63/306,722, filed on Feb. 4, 2022.

(51) Int. Cl. A61K 31/7024; A61K 31/09; A61P 3/02; C07H 15/04

(52) U.S. Cl. CPC A61K 31/7024; A61K 31/09; A61P 3/02; C07H 15/04

(57) ABSTRACT

The present disclosure provides a compound of Formula I: A-R1-A′ wherein A and A′ are independently H or [Formula I], wherein A, A′, and R1 are as described herein. Also provided are methods of making the compounds described herein, and use of the compounds, e.g., in NAD-increasing compositions.

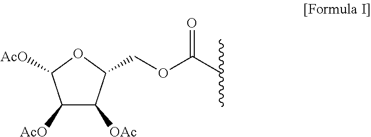 [SMILES: *C(=O)OC[C@H]1O[C@@H](OC(C)=O)[C@H](OC(C)=O)[C@@H]1OC(C)=O]

### FIELD OF THE INVENTION

The present disclosure provides a compound of Formula I.

A-R1-A′ [Formula I]

wherein A and A′ are independently H or

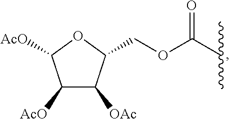

wherein A, A′, and R1 are as described herein. Also provided are methods of making the compounds described herein, and use of the compounds, e.g., in NAD-increasing compositions.

### BACKGROUND

Research in the field of aging, including metabolic dysfunction and metabolic aging, has provided insight into the various roles that nicotinamide adenine dinucleotide (NAD⁺) plays as patients age and diseases progress. From these findings it is known that NAD⁺ decreases as humans get older, and it is desirable to find mechanisms or treatments to maintain NAD⁺ levels to provide increased health benefits.

Nicotinamide riboside and derivatives thereof, including nicotinate riboside, the reduced form of nicotinamide riboside, nicotinamide mononucleotide and nicotinate mononucleotide, are precursors of nicotinamide adenine dinucleotide (NAD⁺) and of its reduced form NADH. Together NADH and NAD⁺ are abbreviated NAD. As a NAD⁺ precursor, nicotinamide riboside has been shown in mice to enhance oxidative metabolism and protect against high-fat diet induced obesity, inflammation, etc., which has resulted in significant interest in nicotinamide riboside and its derivatives. The reduced form of nicotinamide riboside was also shown to dramatically increase NAD levels in animal models. Since nicotinamide riboside is a naturally occurring compound, nicotinamide riboside and its derivatives have great potential as natural, nutritional supplements, which may provide health benefits without causing side effects. Nicotinamide riboside and its derivatives are hydrophilic and require an equilibrative transporter to cross tissues and mammalian cell membranes. Nicotinamide riboside is also a substrate for a ubiquitous hydrolytic enzyme, purine phosphorylase, and is readily metabolized by microbial phosphorylases, thus reducing its oral availability. The reduced form of nicotinamide riboside does not undergo these degradation processes and therefore has the potential to increase the overall NAD boosting upon oral ingestion. The same is true for the acid, nicotinic acid riboside, and the phosphorylated species. However, another approach to decrease the hydrolytic decay of nicotinamide riboside is to prevent enzymatic degradation while facilitating cellular uptake for incorporation in the NAD biosynthetic pathways. Modification of the riboside moiety provide such opportunity. However, such modification must not only provide means to by-pass degradation processes to allow intracellular NAD boosting, but it must also offer high payload, increase energetic capacity, and provide means to manipulate the physicochemical properties of the highly polar species in order to provide intracellular availability despite transport limitations.

### SUMMARY OF THE INVENTION

The present disclosure provides a compound of Formula I:

A-R1-A′ [Formula I]

wherein A and A′ are independently H or

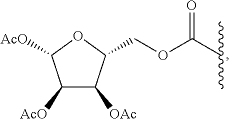

wherein A, A′, and R1 are as described herein.

The present disclosure further provides a compound of Formula II:

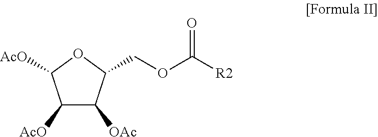

wherein R2 is a substituted or unsubstituted C₈₋₂₄ alkyl, C₈₋₂₄ alkenyl, aryl, alkylaryl, alkenylaryl, or PEG ester alkyl, and Ac is an acetyl group.

The present disclosure further provides a compound of Formula III:

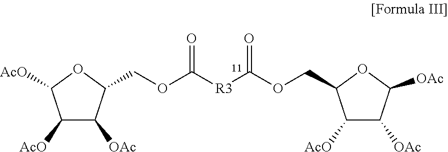

wherein R3 is a substituted or unsubstituted C₁₋₄ alkyl, C₂₋₄ alkenyl, or C₁₋₃ alkyl carboxylate optionally substituted with a protected or a free amine, or wherein R3 comprises a carbon bonded to (i) a hydroxyl group and (ii) one of

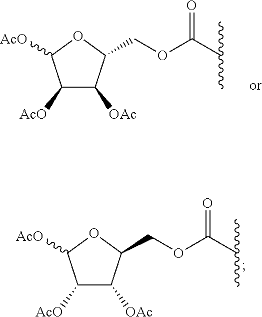 [SMILES: *C(=O)OC[C@H]1OC(OC(C)=O)[C@H](OC(C)=O)[C@@H]1OC(C)=O ; *C(=O)OC[C@@H]1OC(OC(C)=O)[C@@H](OC(C)=O)[C@H]1OC(C)=O]

and
Ac is an acetyl group.

The compounds provided herein can be conjugated, e.g., to a nucleobase and/or to a nicotinamide or a salt, solvate, or derivative thereof, such as dihydronicotinamide, nicotinic acid, nicotinic acid ester, or a reduced form thereof.

Also provided are methods of making and using the compounds described herein, e.g., in a NAD-increasing composition.

### BRIEF DESCRIPTION OF THE DRAWINGS

The following drawings form part of the present specification and are included to further demonstrate exemplary embodiments of certain aspects of the present invention.

FIG. 1A, FIG. 1B, and FIG. 1C respectively show exemplary ¹H NMR, ¹³C NMR, and mass spectra of compound 4 in Synthetic Scheme 1 of Example 1.

FIG. 2A, FIG. 2B, and FIG. 2C respectively show exemplary ¹H NMR, ¹³C NMR, and mass spectra of compound 6a as described in Example 2.

FIG. 3A, FIG. 3B, and FIG. 3C respectively show exemplary ¹H NMR, ¹³C NMR, and mass spectra of compound 6b as described in Example 2.

FIG. 4A, FIG. 4B, and FIG. 4C respectively show exemplary ¹H NMR, ¹³C NMR, and mass spectra of compound 6c as described in Example 2.

FIG. 5A, FIG. 5B, and FIG. 5C respectively show exemplary ¹H NMR, ¹³C NMR, and mass spectra of compound 6d as described in Example 2.

FIG. 6A, FIG. 6B, and FIG. 6C respectively show exemplary ¹H NMR, ¹³C NMR, and mass spectra of compound 6e as described in Example 2.

FIG. 7A, FIG. 7B, and FIG. 7C respectively show exemplary ¹H NMR, ¹³C NMR, and mass spectra of compound 6f as described in Example 2.

FIG. 8A, FIG. 8B, and FIG. 8C respectively show exemplary ¹H NMR, ¹³C NMR, and mass spectra of compound 6g as described in Example 2.

FIG. 9A and FIG. 9B respectively show exemplary ¹H NMR and ¹³C NMR spectra of compound 6h as described in Example 2.

FIG. 10A, FIG. 10B, and FIG. 10C respectively show exemplary ¹H NMR, ¹³C NMR, and ¹⁹F NMR spectra of compound 8a as described in Example 3.

FIG. 11A, FIG. 11B, and FIG. 11C respectively show exemplary ¹H NMR, ¹³C NMR, and mass spectra of compound 8b as described in Example 3.

FIG. 12A, FIG. 12B, and FIG. 12C respectively show exemplary ¹H NMR, ¹³C NMR, and ¹⁹F NMR spectra of compound 8c as described in Example 3.

FIG. 13A, FIG. 13B, and FIG. 13C respectively show exemplary ¹H NMR, ¹³C NMR, and ¹⁹F NMR spectra of compound 8d as described in Example 3.

FIG. 14 shows exemplary ¹H NMR spectra of compound 8e as described in Example 3.

FIG. 15A, FIG. 15B, and FIG. 15C respectively show exemplary ¹H NMR, ¹³C NMR, and ¹⁹F NMR spectra of compound 8f as described in Example 3.

FIG. 16A, FIG. 16B, and FIG. 16C respectively show exemplary ¹H NMR, ¹³C NMR, and ¹⁹F NMR spectra of compound 8g as described in Example 3.

FIG. 17 shows exemplary ¹H NMR spectra of compound 8h as described in Example 3.

FIG. 18A, FIG. 18B, and FIG. 18C respectively show exemplary ¹H NMR, ¹³C NMR, and mass spectra of compound 9b as described in Example 4.

FIG. 19 shows exemplary ¹H NMR spectra of compound 6i as described in Example 6.

FIG. 20 shows exemplary ¹H NMR spectra of compound 8i as described in Example 6.

FIG. 21 shows exemplary ¹H NMR spectra of compound 17 as described in Example 7.

FIGS. 22A-22G show exemplary ¹H NMR spectra of compounds 6(a-g), respectively, as described in Example 1.

FIG. 23A shows exemplary ¹H NMR spectra of compound 8g prior to column chromatography, as described in Example 2. FIGS. 23B and 23C show exemplary ¹H NMR and ¹³C NMR spectra, respectively, of compound 8g, following column chromatography as described in Example 2.

FIGS. 24A-33 relate to Example 5. FIGS. 24A and 24B show exemplary ¹H NMR and ¹⁹F NMR spectra, respectively, of NRTA triflate before ion-exchange.

FIGS. 25A and 25B show exemplary ¹H NMR and ¹⁹F NMR spectra, respectively, of NRTA-chloride after ion exchange (top panels), before ion exchange (middle panels), and during ion exchange (bottom panels).

FIGS. 26A and 26B show exemplary ¹H NMR and ¹⁹F NMR spectra, respectively, of compound 10g (NRLR-Cl) following column purification and ion exchange.

FIG. 27 shows exemplary ¹⁹F NMR spectra of NRLR in D₂O before (bottom panel) and NRLR-Cl in D₂O after (top panel) ion exchange chromatography.

FIG. 28 shows exemplary ¹H NMR spectra of NRLR-Cl after column and ion exchange (top panel), NRLR after column purification (middle panel), and NRLR before column purification (bottom panel).

FIG. 29 shows exemplary ¹H NMR spectra of NRLR without column chromatography (“crude NRLR”).

FIG. 30 shows exemplary ¹H NMR spectra of crude NRLR before (top panel) and after (bottom panel) ion exchange.

FIGS. 31A and 31B show exemplary ¹⁹F NMR spectra and mass spectra, respectively, of crude NRLR after ion exchange.

FIG. 32 shows exemplary ¹⁹F NMR spectra of crude NRLR before (top panel) and after (bottom panel) ion exchange.

FIGS. 33A, 33B, 33C, and 33D show exemplary ¹H NMR, ¹⁹F NMR, ¹³C NMR, and HSQC spectra, respectively, of compound 10g after the reverse phase column chromatography.

FIGS. 34A-39 relate to Example 9. FIG. 34A shows exemplary ¹H NMR spectra of a purine nucleoside phosphorylase (PNP) enzyme activity assay with NR.

FIG. 34B shows exemplary ¹H NMR spectra of a purine nucleoside phosphorylase (PNP) enzyme activity assay with NR and NRTA. The top panel shows NR after 5 minutes of PNP addition. The second, third, and fourth panels show NRTA after 20 minutes of PNP addition, 0 minutes of PNP addition, and without PNP, respectively.

FIG. 34C shows exemplary ¹H NMR spectra of a PNP assay with a NRTA over a longer time course at the indicated time points: 24 hours, 20 minutes, or 5 minutes after PNP addition, or before PNP addition.

FIG. 35 shows exemplary ¹H NMR spectra of NRLR-triflate in 100% DMSO.

FIG. 36 shows exemplary ¹H NMR spectra of a lipase assay with NRLR at the indicated time points: 12 hours, 6 hours, 1 hour, 30 minutes, or 5 minutes after lipase addition.

FIG. 37 shows exemplary ¹H NMR spectra of a lipase assay with NRLR and higher concentration of lipase at the indicated time points: 12 hours, 6 hours, and 5 minutes after lipase addition.

FIG. 38 shows exemplary ¹H NMR spectra of a PNP assay on NRLR at the indicated time points: 24 hours, 18 hours, 12 hours, 6 hours, 1 hour, or 5 minutes after PNP addition, or before PNP addition.

FIG. 39 shows exemplary ¹H NMR spectra of a PNP assay on NR adipate at the indicated time points: 18 hours, 12 hours, 6 hours, 1 hour, 30 minutes, 15 minutes, or 5 minutes after PNP addition, or before PNP addition.

FIG. 40A shows exemplary ¹H NMR spectra of a PNP assay on NR adipate, NR malonate, NR laurate, and NR-chloride after 1 day of incubation with PNP. FIG. 40A shows exemplary ¹H NMR spectra of a PNP assay on NR adipate, NR malonate, NR laurate, and NR-chloride after 2 days of incubation with PNP.

### DETAILED DESCRIPTION OF THE INVENTION

The present disclosure provides novel ribose linker compounds, which may be functionalized with biologically active agents of interest or prodrugs thereof, of Formula I:

A-R1-A′ [Formula I]

wherein A and A′ are independently H or

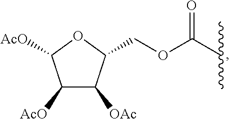

wherein A, A′, and R1 are as described herein.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y and any numbers that fall within x and y.

The term “Ac” refers to acetyl.

The term “alkyl” means an acyclic alkyl moiety that is linear or branched, preferably containing more than 1 carbon atom, e.g., about 2 to about 50 carbon atoms, or about 4 to about 40 carbon atoms, or about 6 to about 30 carbon atoms, or about 8 to about 24 carbon atoms. Said alkyl moiety can be optionally substituted with groups as described herein, e.g., halo, hydroxyl, amino (e.g., primary, secondary, or tertiary amino), carboxyl, carboxyalkyl, methoxy, ethoxy, alkoxyamino, alkoxyamido, trifluoromethyl, sulfonyl, sulfonamido, acetamido, cyano, nitro, or any combination thereof.

The term “alkenyl” refers to an unsaturated, acyclic hydrocarbon moiety that is linear or branched and that contains at least one double bond, e.g., 1, 2, 3, 4, 5, or more than 5 double bonds, and preferably containing about 2 to about 50 carbon atoms, or about 4 to about 40 carbon atoms, or about 6 to about 30 carbon atoms, or about 8 to about 24 carbon atoms. Said alkenyl moiety can be optionally substituted with groups as described herein, e.g., halo, hydroxyl, amino, amido, carboxyl, carboxyalkyl, methoxy, ethoxy, alkoxyamino, alkoxyamido, trifluoromethyl, sulfonyl, sulfonamido, acetamido, cyano, nitro, azido, or any combination thereof. Examples of suitable alkenyl moieties include octen-1-yl, nonen-1-yl, decen-1-yl, stearyl, oleyl, linoleyl, linolenyl, arachidonyl, eicasopentaenyl, and docosahexaenyl.

The term “alkynyl” refers to an unsaturated, acyclic hydrocarbon moiety that is linear or branched and that contains at least one triple bonds, e.g., 1, 2, 3, 4, 5, or more than 5 triple bonds, and preferably containing about 2 to about 50 carbon atoms, or about 4 to about 40 carbon atoms, or about 6 to about 30 carbon atoms, or about 8 to about 24 carbon atoms. Said alkynyl moiety can be optionally substituted with groups as described herein, e.g., halo, hydroxyl, amino (e.g., primary, secondary, or tertiary amino), carboxyl, carboxyalkyl, methoxy, ethoxy, alkoxyamino, alkoxyamido, trifluoromethyl, sulfonyl, sulfonamido, acetamido, cyano, nitro, or any combination thereof.

The term “alkoxy” includes linear or branched oxy-containing moieties, each having an alkyl portion as described above, e.g., having about 2 to about 50 carbon atoms, or about 4 to about 40 carbon atoms, or about 6 to about 30 carbon atoms, or about 8 to about 24 carbon atoms. The term “alkoxyalkyl” includes alkyl moieties having one or more alkoxy moieties attached to the alkyl moiety.

The term “aryl” means a fully unsaturated mono- or multi-ring carbocycle. Examples of such moieties include substituted or unsubstituted phenyl (or benzyl), naphthyl, and anthracenyl. The term “aryl,” as used alone or within other terms, means a mono- or multi-ring aromatic ring structure containing one to four rings, wherein such rings may be attached together in a pendent manner or may be fused. Such an aryl group may have one or more substituents such as, but not limited to, alkyl, hydroxy, halo, haloalkyl, amino, nitro, cyano, alkoxy, and alkylamino. The term “aryl” refers to both cyclic structures consisting only of carbons (carboaryls) and cyclic structures comprising carbon and a heteroatom, e.g., selected from the group consisting of nitrogen, sulfur, and oxygen (heteroaryls). The term “aryl” also includes polycyclic heteroaryls, e.g., indole, phthalide (benzofuran), 1H-indazole, and 1H-pyrrolo[2,3-b]pyridine, indoline, tetrahydroquinoline, and 2,3-dihydrobenzofuran.

The term “BOC” or “Boc” refers to tert-butoxycarbonyl.

The term “prodrug” means a chemical derivative of an active parent drug that, upon spontaneous or enzymatic biotransformation, releases the active parent drug. The term “prodrug” includes variations or derivatives of the compounds of this invention that have groups cleavable under metabolic conditions, including solvolysis or enzymatic degradation. In some embodiments, the prodrug is pharmacologically inactive or exhibits reduced activity relevant to its active parent drug.

The term “substituted” means that any one or more hydrogen atoms is replaced with any suitable substituent, provided that the normal valency is not exceeded and the replacement results in a stable compound. Suitable substituents include, but are not limited to, alkyl, alkylaryl, aryl, heteroaryl, halide, hydroxyl, carboxylate, carbonyl (including alkylcarbonyl and arylcarbonyl), phosphate, amino (including alkylamino, dialkylamino, hydroxylamino, dihydroxylamino, alkyl hydroxylamino, arylamino, diarylamino and alkylarylamino), thiol (including alkylthiol, arylthiol and thiocarboxylate), sulfate, nitro, cyano and azido.

### Ribose Monomers

The present disclosure provides novel ribose linker compounds, which may be functionalized with biologically active agents of interest or prodrugs thereof, of Formula I:

A-R1-A′ [Formula I]

wherein A and A′ are independently H or

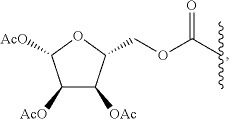

wherein A and A′ are not both H, and:
when one of A or A′ is H, R1 is a substituted or unsubstituted C₈₋₂₄ alkyl, C₈₋₂₄ alkenyl, aryl, alkylaryl, or alkenylaryl; or
when A and A′ are both not H, R1 is a substituted or unsubstituted C₁₋₄ alkyl, C₂₋₄ alkenyl, polyethylene glycol (PEG) ester alkyl, or C₁₋₃ alkyl carboxylate optionally substituted with a protected or free amine; and
Ac is an acetyl group.

In some embodiments, one of A or A′ of Formula I is hydrogen. Thus, in some embodiments, the compound of Formula I is:

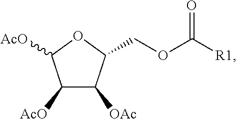

for example, a compound of Formula II

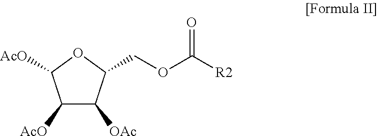

Suitably, the R1 of Formula I or the R2 of Formula II is a substituted or unsubstituted C₈₋₂₄ alkyl, C₈₋₂₄ alkenyl, aryl, alkylaryl, alkenylaryl, or PEG ester alkyl.

Suitably, the R1 of Formula I or the R2 of Formula II is an unsubstituted C₂₋₄₀ alkyl, or an unsubstituted C₄₋₃₆ alkyl, or an unsubstituted C₆₋₃₀ alkyl, or an unsubstituted C₈₋₂₄ alkyl. For example, R1 or R2 and the oxycarbonyl (O—C═O) to which it is bonded can together form a saturated fatty acid ester group, e.g., an ester of caprylic acid (“capryloyl”; C₈), capric acid (“caproyl”; C₁₀), lauric acid (“lauroyl”; C₁₂), myristic (“myristoyl”; C₁₄), palmitic acid (“palmitoyl”; C₁₆), stearic acid (“stearoyl”; C₁₈), arachidic acid (“arachidoyl”; CM), behenic acid (“behenoyl”; C₂₂), or tetracosanoic acid (“tetracosanoyl”; C₂₄). In certain embodiments, R1 or R2 is a C₁₂ alkyl.

In some embodiments, the compound of Formula I or Formula II is:

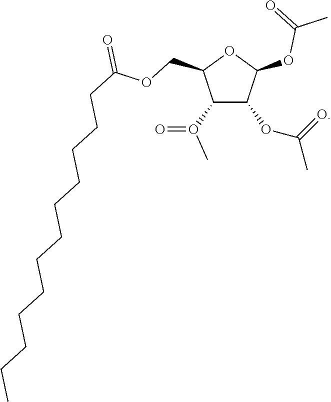

In further embodiments, the R1 of Formula I or the R2 of Formula II is an unsubstituted C₂₋₄₀ alkenyl, or an unsubstituted C₄₋₃₆ alkenyl, or an unsubstituted C₆₋₃₀ alkenyl, or an unsubstituted C₈₋₂₄ alkenyl. For example, R1 or R2 and the oxycarbonyl (O—C═O) to which it is bonded can together form an unsaturated fatty acid ester group, e.g., an ester of myristoleic acid (“myristoleoyl”), palmitoleic acid (“palmitoleoyl”), sapienic acid (“sapienoyl”), oleic acid (“oleoyl”), elaidic acid (“elaidoyl”), vaccenic acid (“vaccenoyl”), linoleic acid (“linoleoyl”), linoelaidic acid (“linoelaoyl”), alpha-linolenic acid (“alpha-linolenoyl”), gamma-linolenic acid (“gamma-linolenoyl”), arachidonic acid (“arachidonoyl”), eicosapentaenoic acid (“eicosapentaenoyl”), erucic acid (“erucoyl”), or docosahexaenoic acid (“docosahexaenoyl”). In some embodiments, R1 or R2 and the oxycarbonyl to which it is bonded forms an ester of an essential fatty acid, e.g., an ester of oleic acid, linoleic acid, or alpha-linolenic acid. In further embodiments, R1 or R2 and the oxycarbonyl to which it is bonded forms an ester of an omega-3 fatty acid, e.g., alpha-linolenic acid, eicosapentaenoic acid, or docosahexaenoic acid.

In yet further embodiments, the R1 of Formula I or the R2 of Formula II is a substituted alkyl, e.g., C₂₋₄₀ alkyl, C₄₋₃₆ alkyl, C₆₋₃₀ alkyl, or C₈₋₂₄ alkyl as described herein, or a substituted alkenyl, e.g., C₂₋₄₀ alkenyl, C₄₋₃₆ alkenyl, C₆₋₃₀ alkenyl, or C₈₋₂₄ alkenyl as described herein. Suitable alkyl and alkenyl substituents are known to one of ordinary skill in the art. As non-limiting examples, the alkyl or alkenyl can be substituted, at any one or more positions along the alkyl or alkenyl chain, with one or more of an alkyl, alkylaryl, aryl, heteroaryl, halide, hydroxyl, carboxylate, carbonyl, alkylcarbonyl, arylcarbonyl, phospho, amino, alkylamino, dialkylamino, hydroxylamino, dihydroxylamino, alkylhydroxylamino, arylamino, diarylamino and alkylarylamino, thiol, alkylthiol, arylthiol, thiocarboxylate, sulfate, nitro, cyano, azido, or any combination thereof.

In still further embodiments, the R1 of Formula I or the R2 of Formula II is an aryl, alkylaryl, or alkenylaryl. For example, R1 or R2 can be a substituted or unsubstituted phenyl or benzyl. Suitably, the substitution may be at any position in the aryl. Suitably, the substitution comprises an alkyl, alkylaryl, aryl, heteroaryl, halide, hydroxyl, carboxylate, carbonyl, alkylcarbonyl, arylcarbonyl, phospho, amino, alkylamino, dialkylamino, hydroxylamino, dihydroxylamino, alkylhydroxylamino, arylamino, diarylamino and alkylarylamino, thiol, alkylthiol, arylthiol, thiocarboxylate, sulfate, nitro, cyano, and azido. In certain embodiments, R1 or R2 is a substituted benzyl, for example, an amino-substituted benzyl. In a specific embodiment, R1 or R2 is a para-aminobenzyl. In further embodiments, R1 or R2 is an alkylaryl or alkenylaryl, for example, a C₁₋₂₄ alkyl or C₁₋₂₄ alkenyl and an aryl group described herein, e.g., a phthalide.

In still further embodiments, the R1 of Formula I or the R2 of Formula II comprises a pharmaceutically active agent. Suitably, R1 or R2 comprises a vitamin, e.g., retinol. Suitably, R1 or R2 and the oxycarbonyl to which it is bonded forms a succinate ester of retinol. In further embodiments, R1 or R2 and the oxycarbonyl to which it is bonded forms a mycophenolate ester. Thus, in embodiments, the compound of Formula I or Formula II is:

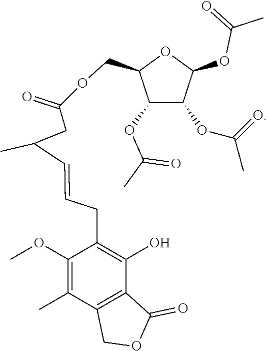 [SMILES: COc1c(C)c2c(c(O)c1C/C=C/C(C)CC(=O)OC[C@H]1O[C@@H](OC(C)=O)[C@H](OC(C)=O)[C@@H]1OC(C)=O)C(=O)OC2]

In additional embodiments, the R1 of Formula I or the R2 of Formula II is an alkyl polyethylene glycol (PEG) ester, i.e., the R1 or R3 has the structure of —O(CH₂)—ₘO(CH₂CH₂O)ₙCH₃, wherein m is an integer from 1 to 10, and n is an integer from 5 to 200. In embodiments, the alkyl PEG ester comprises about 5 to about 200 ethylene glycol units, or about 8 to about 150 ethylene glycol units, or about 10 to about 100 ethylene glycol units, or about 20 to about 80 ethylene glycol units, or about 30 to about 70 ethylene glycol units, or about 40 to about 60 ethylene glycol units. In some embodiments, the compound of Formula I or Formula II is:

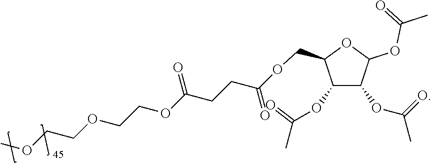

### Ribose Dimers

In certain embodiments, neither of A and A′ of Formula I is hydrogen. In such embodiments, the compound of Formula I is:

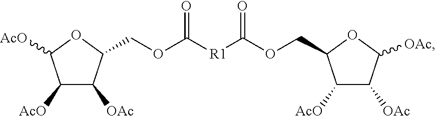

for example, a compound of Formula III:

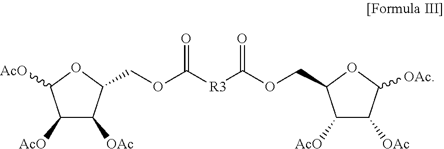

In embodiments, the compound of Formula III is any one of:

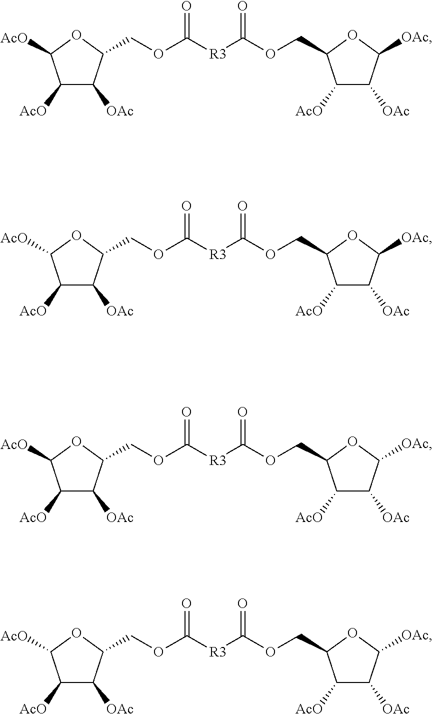

or a mixture thereof.

Suitably, the R1 of Formula I or the R3 of Formula III is a substituted or unsubstituted C₁₋₄ alkyl, C₁₋₄ alkenyl, polyethylene glycol (PEG) ester alkyl, or C₁₋₃ alkyl carboxylate optionally substituted with a protected or free amine.

Suitably, the R1 of Formula I or the R3 of Formula III is an unsubstituted C₁₋₁₀ alkyl, or an unsubstituted C₁₋₈ alkyl, or an unsubstituted C₁₋₆ alkyl, or an unsubstituted C₁₋₄ alkyl. For example, R1 or R3 can be —CH₂—, —CH₂CH₂—, —CH₂CH₂CH₂—, or —CH₂CH₂CH₂CH₂—.

In some embodiments, the R1 or R3 and the oxycarbonyl groups to which it is bonded together form a malonic diester (R1 or R3 is —CH₂—); a succinic diester (R1 or R3 is (—CH₂CH₂—); a glutaric diester (R1 or R3 is —CH₂CH₂CH₂—); or an adipic diester (R1 or R3 is —CH₂CH₂CH₂CH₂—).

For example, the compound of Formula I or Formula III can be any one of:

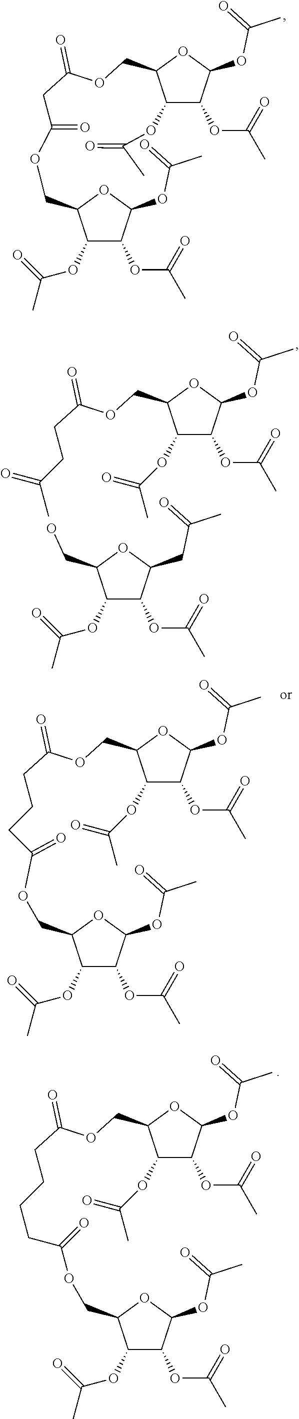

In further embodiments, the R1 of Formula I or the R3 of Formula III is a substituted alkyl, e.g., C₁₋₁₀ alkyl, C₁₋₈ alkyl, C₁₋₆ alkyl, or C₁₋₄ alkyl as described herein. As non-limiting examples, the alkyl can be substituted, at any one or more positions along the alkyl chain, with one or more of an alkyl, alkylaryl, aryl, heteroaryl, halide, hydroxyl, carboxylate, carbonyl, alkylcarbonyl, arylcarbonyl, phospho, amino, alkylamino, dialkylamino, hydroxylamino, dihydroxylamino, alkylhydroxylamino, arylamino, diarylamino and alkylarylamino, thiol, alkylthiol, arylthiol, thiocarboxylate, sulfate, nitro, cyano, azido, or any combination thereof. Suitably, R1 or R3 is an alkyl substituted with an amino group. The amino group may comprise a protected or unprotected amine (also referred to herein as a “free” amine). In certain embodiments, R1 or R3 is an alkyl (e.g., C₁₋₃ alkyl) substituted with a Boc-protected amine. In further embodiments, R1 or R3 is an alkyl (e.g., C₁₋₃ alkyl) substituted with an N-Boc-protected glutamate or aspartate. In still further embodiments, R1 or R3 is an alkyl (e.g., C₁₋₃ alkyl) substituted with

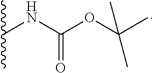 [SMILES: *NC(=O)OC(C)(C)C]

In certain embodiments, the compound of Formula I or Formula III is:

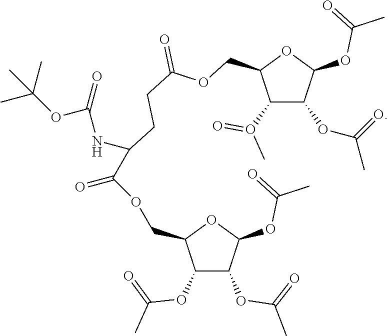

Suitably, the R1 of Formula I or the R3 of Formula III is an unsubstituted C₂₋₁₀ alkenyl, or an unsubstituted C₂₋₈ alkenyl, or an unsubstituted C₂₋₆ alkenyl, or an unsubstituted C₂₋₄ alkenyl, wherein the alkenyl comprises one or more double bonds. For example, R1 or R3 can be —CH═CH—, —CH═CHCH₂—, —CH₂CH═CH—, —CH═CHCH₂CH₂—, —CH₂CH═CHCH₂—, or —CH₂CH₂CH═CH—. In a further example, R1 or R3 can include multiple double bonds, which may be located at any position within the alkenyl chain. For example, R1 or R3 can be —CH═C═CH—, —CH═C═CHCH₂—, —CH₂CH═C═CH—, or —CH═C═C═CH—. Suitably, the R1 or R3 and the oxycarbonyl groups to which it is bonded together form a fumarate ester or a maleate ester. In an embodiment, the compound of Formula I or Formula III is:

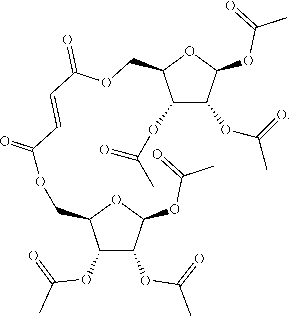 [SMILES: CC(=O)O[C@@H]1O[C@H](COC(=O)/C=C/C(=O)OC[C@H]2O[C@@H](OC(C)=O)[C@H](OC(C)=O)[C@@H]2OC(C)=O)[C@@H](OC(C)=O)[C@H]1OC(C)=O]

In further embodiments, the R1 of Formula I or the R3 of Formula III is a substituted alkenyl. e.g., C₂₋₁₀ alkenyl, C₂₋₈ alkenyl, C₂₋₆ alkenyl, or C₂₋₄ alkenyl as described herein. As non-limiting examples, the alkenyl can be substituted, at any one or more positions along the alkenyl chain, with one or more of an alkyl, alkylaryl, aryl, heteroaryl, halide, hydroxyl, carboxylate, carbonyl, alkylcarbonyl, arylcarbonyl, phospho, amino, alkylamino, dialkylamino, hydroxylamino, dihydroxylamino, alkylhydroxylamino, arylamino, diarylamino and alkylarylamino, thiol, alkylthiol, arylthiol, thiocarboxylate, sulfate, nitro, cyano, azido, or any combination thereof.

In still further embodiments, the R1 of Formula I or the R3 of Formula III is an alkyl PEG ester, i.e., the R1 or R3 has the structure of —O(CH₂)ₘO(CH₂CH₂O)ₙ, wherein m is an integer from 1 to 10, and n is an integer from 5 to 200. In embodiments, the alkyl PEG ester comprises about 5 to about 200 ethylene glycol units, or about 8 to about 150 ethylene glycol units, or about 10 to about 100 ethylene glycol units, or about 20 to about 80 ethylene glycol units, or about 30 to about 70 ethylene glycol units, or about 40 to about 60 ethylene glycol units. In some embodiments, the compound of Formula I or Formula III is:

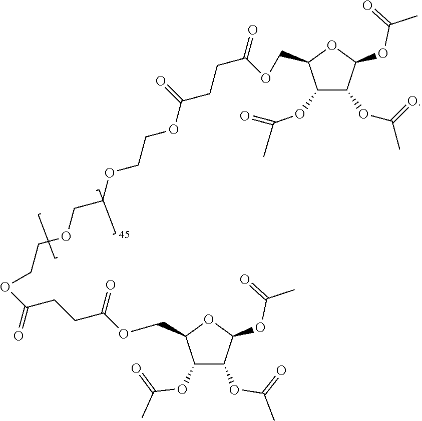

### Ribose Trimers

In further embodiments, R3 of the compound of Formula III comprises a carbon bonded to (i) a hydroxyl group and (ii) one of

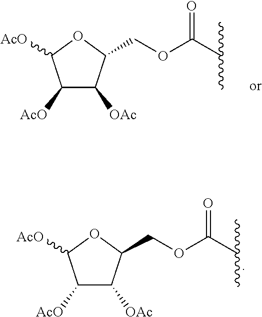 [SMILES: *C(=O)OC[C@H]1OC(OC(C)=O)[C@H](OC(C)=O)[C@@H]1OC(C)=O ; *C(=O)OC[C@@H]1OC(OC(C)=O)[C@@H](OC(C)=O)[C@H]1OC(C)=O]

Suitably, the compound of Formula III is:

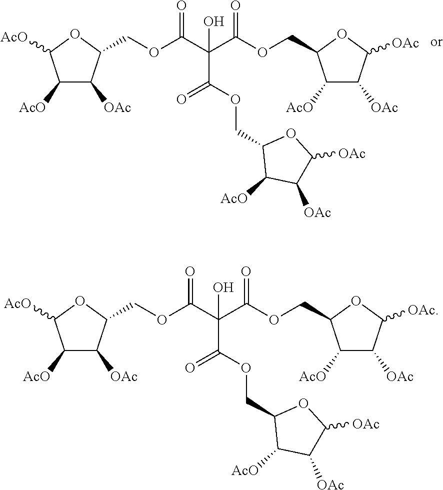 [SMILES: CC(=O)OC1O[C@@H](COC(=O)C(O)(C(=O)OC[C@H]2OC(OC(C)=O)[C@H](OC(C)=O)[C@@H]2OC(C)=O)C(=O)OC[C@H]2OC(OC(C)=O)[C@H](OC(C)=O)[C@@H]2OC(C)=O)[C@H](OC(C)=O)[C@@H]1OC(C)=O ; CC(=O)OC1O[C@H](COC(=O)C(O)(C(=O)OC[C@H]2OC(OC(C)=O)[C@H](OC(C)=O)[C@@H]2OC(C)=O)C(=O)OC[C@H]2OC(OC(C)=O)[C@H](OC(C)=O)[C@@H]2OC(C)=O)[C@@H](OC(C)=O)[C@H]1OC(C)=O]

In embodiments the compound of Formula III is any of

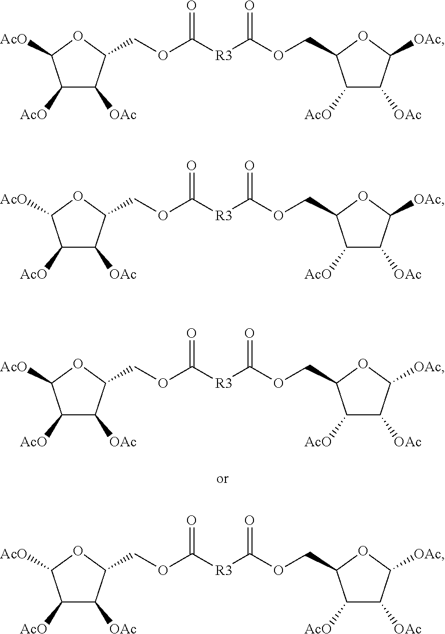

wherein R3 is a carbon bonded to (i) a hydroxyl group and (ii)

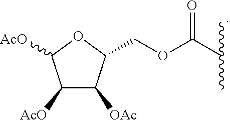 [SMILES: *C(=O)OC[C@H]1OC(OC(C)=O)[C@H](OC(C)=O)[C@@H]1OC(C)=O]

In embodiments, the compound of Formula III is any of

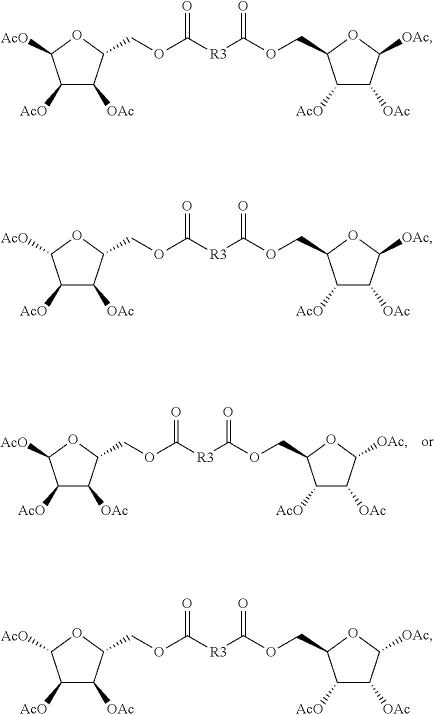

wherein R3 is a carbon bonded to (i) a hydroxyl group and (ii)

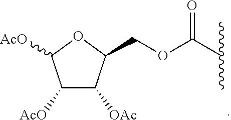 [SMILES: *C(=O)OC[C@@H]1OC(OC(C)=O)[C@@H](OC(C)=O)[C@H]1OC(C)=O]

### Nucleoside Conjugates

In some embodiments, the ribose linker compounds provided herein, e.g., compounds of Formula I, Formula II, or Formula III, are coupled to one or more additional compounds. The one or more additional compounds can be a biologically active agent, for example a therapeutic agent. Suitably, the biologically active agent is a nucleobase. As used herein, “nucleobase” includes all naturally occurring nucleobases typically found in DNA and RNA, e.g., adenine, cytosine, guanine, thymine, and uracil; modified nucleobases, such as hypoxanthine, xanthine, 7-methylguanine, 5,6-dihydrouracil, 5-methylcytosine, and 5-hydroxymethylcytosine; artificial nucleobases, also referred to as nucleobase analogues, such as isoguanine and isocytosine; and pyridine-nucleobases, e.g., nicotinamide and/or a salt, solvate, or derivative thereof, such as dihydronicotinamide, nicotinic acid, and nicotinic acid ester, and reduced forms thereof.

Suitably, the one or more additional compounds, e.g., nucleobase, are attached to the ribose of Formula I at the 1′ carbon of the ribose ring, e.g., to provide a compound of Formula IV, wherein the one or more additional compounds is depicted as R4:

B—R1-B′ [Formula IV]

wherein B and B′ are independently H or

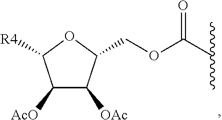

wherein Ac is an acetyl group; B and B′ are not both H; and R1 is as described herein for Formula I. In some embodiments, when one of B or B′ of Formula IV is H, R1 is a substituted or unsubstituted C₈₋₂₄ alkyl, C₈₋₂₄ alkenyl, aryl, alkylaryl, or alkenylaryl, as described herein. In further embodiments, when B and B′ are both not H, R1 is a substituted or unsubstituted C₁₋₄ alkyl, C₁₋₄ alkenyl, PEG ester alkyl, or C₁₋₃ alkyl carboxylate optionally substituted with a protected or free amine, as described herein.

Suitably, the one or more additional compounds, e.g., nucleobase, are attached to the ribose of Formula II at the 1′ carbon of the ribose ring, e.g., to provide a compound of Formula VI, wherein the one or more additional compounds is depicted as R4:

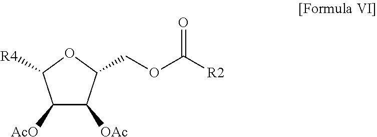

wherein Ac is an acetyl group, and R2 is as described herein for Formula II. In some embodiments, R2 is a substituted or unsubstituted C₈₋₂₄ alkyl, C₈₋₂₄ alkenyl, aryl, alkylaryl, alkenylaryl, or PEG ester alkyl, as described herein.

Suitably, the one or more additional compounds, e.g., nucleobase, are attached to the ribose of Formula III at the 1′ carbon of the ribose ring, e.g., to provide a compound of Formula VIII, wherein the one or more additional compounds is depicted as R4 and R4′:

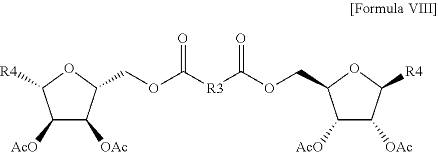

wherein Ac is an acetyl group, and R3 is as described herein for Formula III. In some embodiments, R3 is a substituted or unsubstituted C₁₋₄ alkyl, C₁₋₄ alkenyl, or C₁₋₃ alkyl optionally substituted with a protected or free amine, as described herein.

In further embodiments, R3 comprises a carbon bonded to (i) a hydroxyl group and (ii) one of

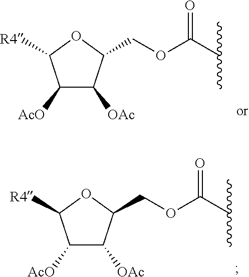

In some embodiments, the compound of Formula VIII is

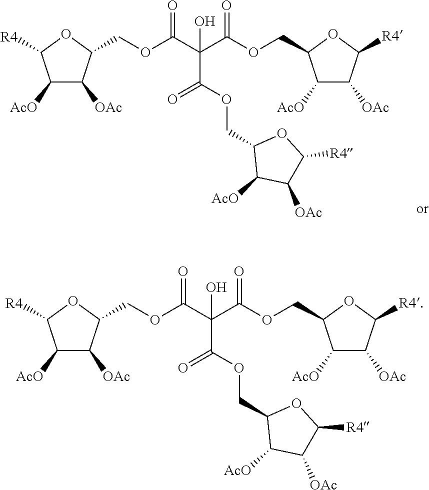

Suitably, R4 of Formula IV, Formula VI, and Formula VIII, R4′ of Formula VIII, and/or R4″ of Formula VIII is a nucleobase. Nucleobases are described herein. In embodiments, R4, R4′, and R4″ are each independently a nucleobase selected from adenine, cytosine, guanine, thymine, uracil; a modified nucleobase selected from hypoxanthine, xanthine, 7-methylguanine, 5,6-dihydrouracil, 5-methyleytosine, and 5-hydroxymethyleytosine; an artificial nucleobase selected from isoguanine and isocytosine; and a pyridine-containing compound selected from nicotinamide, dihydronicotinamide, nicotinic acid, nicotinic acid ester, and a reduced form thereof. In certain embodiments, R4, R4′, and/or R4″ is nicotinamide or a salt, solvate, or derivative thereof, e.g., dihydronicotinamide, nicotinic acid, nicotinic acid ester, or a reduced form thereof. Structures of nicotinamide and related compounds are provided below.

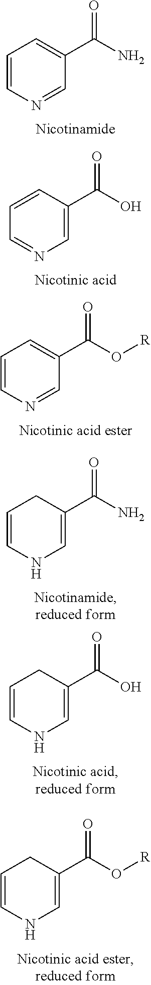

The point of attachment of R4, R4′, and/or R4″ to the ribose of Formula IV, Formula VI, and/or Formula VIII can be determined by one of ordinary skill in the art. Suitably, in embodiments, the nucleobase is conjugated via a nitrogen atom, e.g., the nitrogen at the 1 position of a pyrimidine ring (e.g., cytosine, uracil, thymine, and the like) or a nitrogen at the 9 position of a purine ring (e.g., adenine, guanine, and the like). In embodiments, the nicotinamide and/or related compound described herein are conjugated via the nitrogen of the pyridine or dihydropyridine ring. It will be understood by one of ordinary skill in the art that upon conjugation of the oxidized form of nicotinamide and its derivatives via the nitrogen atom in the ring, the nitrogen atom will carry a +1 charge. It will be further understood by one of ordinary skill in the art that upon conjugation of the reduced form of nicotinamide and its derivatives via the nitrogen atom in the ring, the nitrogen atom in the ring will no longer be bonded to the hydrogen.

Suitably, the nicotinamide or a salt, solvate, or derivative thereof is linked to the compound of Formula IV, VI, or VIII via the nitrogen of the pyridine ring, to provide a nicotinamide ribose (NR) conjugate or a salt, solvate, or derivative thereof.

### Nicotinamide Ribose Conjugates

Nicotinamide riboside (NR) is a precursor to nicotinamide adenine dinucleotide (NAD or NAD+), nicotinamide adenine dinucleotide phosphate (NADP or NADP+), and their respective phosphorylated forms (NADH and NADPH, respectively), all of which are important enzyme cofactors. For example, NAD+ is involved in metabolic processes such as energy production, DNA repair, cellular detoxification, the inflammatory response, and protein folding. The structure of NR is provided below:

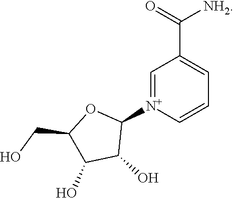 [SMILES: NC(=O)c1ccc[n+]([C@@H]2O[C@H](CO)[C@@H](O)[C@H]2O)c1]

As shown above, NR is a quaternary salt and is capable of forming an ionic bond with a counterion, e.g., a counteranion. Examples of counterions include the anions of suitable organic acid such as formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic, and triflic (i.e., trifluoromethanesulfonic) acids, theophylline acetic acids, and 8-halotheophyllines, e.g., 8-bromotheophylline and the like. Further examples of pharmaceutical acceptable inorganic or organic acid counterions include the pharmaceutically acceptable salts listed in J. Pharm. Sci. 66(1):2 (1977).

Suitably, the R4 in Formula IV is nicotinamide or a derivative thereof, e.g., dihydronicotinamide, nicotinic acid, nicotinic acid ester, or a reduced form thereof, thereby providing a compound of Formula V, wherein R5 is nicotinamide, dihydronicotinamide, nicotinic acid, nicotinic acid ester, or a reduced form thereof:

C—R1-C′ [Formula V]

wherein C and C′ are independently H or

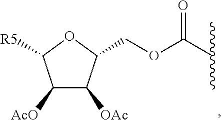

wherein Ac is an acetyl group; C and C′ are not both H; and R1 is as described herein for Formula I. In some embodiments, when one of C or C′ of Formula IV is H, R1 is a substituted or unsubstituted C₈₋₂₄ alkyl, C₈₋₂₄ alkenyl, aryl, alkylaryl, or alkenylaryl, as described herein. In further embodiments, when C and C′ are both not H, R1 is a substituted or unsubstituted C₁₋₄ alkyl, C₁₋₄ alkenyl, PEG ester alkyl, or C₁₋₃ alkyl carboxylate optionally substituted with a protected or free amine, as described herein.

Suitably, the R4 in Formula VI is nicotinamide or a derivative thereof, e.g., dihydronicotinamide, nicotinic acid, nicotinic acid ester, or a reduced form thereof, thereby providing a compound of Formula VII, wherein R5 is nicotinamide, dihydronicotinamide, nicotinic acid, nicotinic acid ester, or a reduced form thereof:

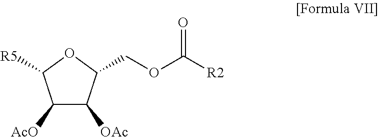

wherein Ac is an acetyl group, and R2 is as described herein for Formula II. In some embodiments, R2 is a substituted or unsubstituted C₈₋₂₄ alkyl, C₈₋₂₄ alkenyl, aryl, alkylaryl, alkenylaryl, or PEG ester alkyl, as described herein.

Suitably, R1 of Formula V or R2 of Formula VII is an unsubstituted C₈₋₂₄ alkyl as described herein. In exemplary embodiments, the compound of Formula V or Formula VII is:

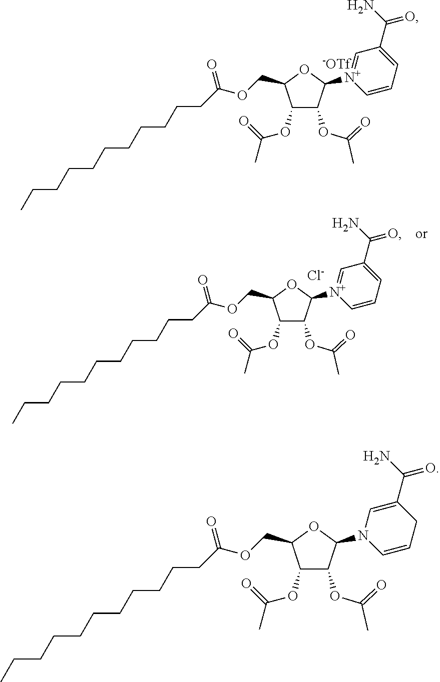 [SMILES: CCCCCCCCCCCC(=O)OC[C@H]1O[C@@H]([n+]2cccc(C(N)=O)c2)[C@H](OC(C)=O)[C@@H]1OC(C)=O ; CCCCCCCCCCCC(=O)OC[C@H]1O[C@@H]([n+]2cccc(C(N)=O)c2)[C@H](OC(C)=O)[C@@H]1OC(C)=O ; [Cl-] ; CCCCCCCCCCCC(=O)OC[C@H]1O[C@@H](N2C=CCC(C(N)=O)=C2)[C@H](OC(C)=O)[C@@H]1OC(C)=O ; "-OTf"]

It will be understood by one of ordinary skill in the art that, while the NR derivative structures depicted herein comprise a triflate counteranion, the triflate may be replaced with any suitable counteranion, e.g., as described herein.

Suitably, R1 of Formula V or R2 of Formula VII comprises a pharmaceutically active agent as described herein, e.g., R1 or R2 and the oxycarbonyl to which it is bonded form a mycophenolate ester. In exemplary embodiments, the compound of Formula V or Formula VII is:

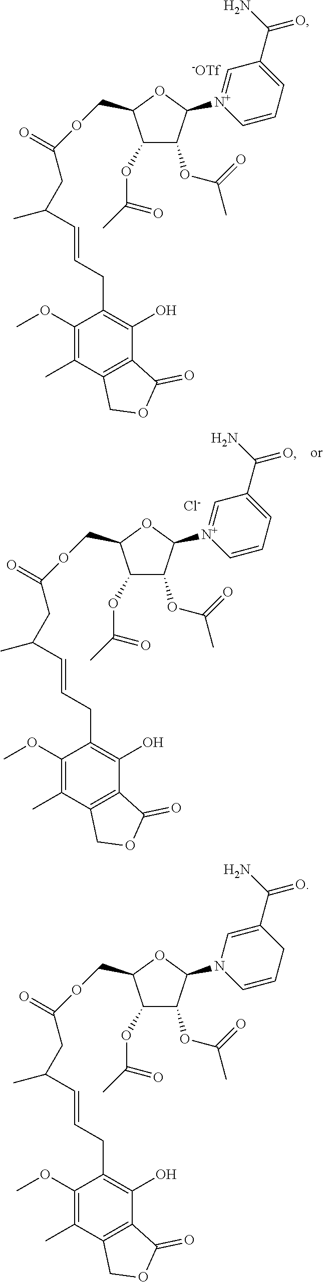 [SMILES: COc1c(C)c2c(c(O)c1C/C=C/C(C)CC(=O)OC[C@H]1O[C@@H]([n+]3cccc(C(N)=O)c3)[C@H](OC(C)=O)[C@@H]1OC(C)=O)C(=O)OC2 ; COc1c(C)c2c(c(O)c1C/C=C/C(C)CC(=O)OC[C@H]1O[C@@H]([n+]3cccc(C(N)=O)c3)[C@H](OC(C)=O)[C@@H]1OC(C)=O)C(=O)OC2 ; [Cl-] ; "-OTf" ; COc1c(C)c2c(c(O)c1C/C=C/C(C)CC(=O)OC[C@H]1O[C@@H](N3C=CCC(C(N)=O)=C3)[C@H](OC(C)=O)[C@@H]1OC(C)=O)C(=O)OC2]

Suitably, R1 of Formula V or R2 of Formula VII is an alkyl PEG ester as described herein, e.g., comprising about 5 to about 200 ethylene glycol units, suitably about 8 to about 150 ethylene glycol units, suitably about 10 to about 100 ethylene glycol units, suitably about 20 to about 80 ethylene glycol units, suitably about 30 to about 70 ethylene glycol units, or suitably about 40 to about 60 ethylene glycol units. In exemplary embodiments, the compound of Formula V or Formula VII is:

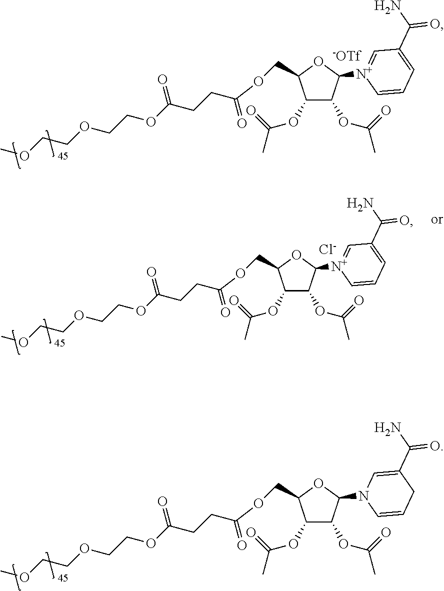

In further embodiments, the R4 is Formula VIII is nicotinamide or a derivative thereof, e.g., dihydronicotinamide, nicotinic acid, nicotinic acid ester, or a reduced form thereof, thereby providing a compound of Formula IX, wherein R5 and R5′ are each independently nicotinamide, dihydronicotinamide, nicotinic acid, nicotinic acid ester, or a reduced form thereof.

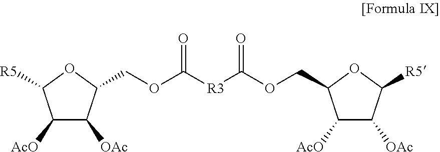

wherein Ac is an acetyl group, and R3 is as described herein for Formula III. In some embodiments, R3 is a substituted or unsubstituted C₁₋₄ alkyl, C₁₋₄ alkenyl, or C₁₋₃ alkyl optionally substituted with a protected or free amine, as described herein.

In further embodiments, R3 comprises a carbon bonded to (i) a hydroxyl group and (ii) one of

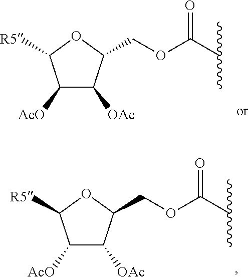

wherein R5, R5′, and R5″ are each independently nicotinamide, dihydronicotinamide, nicotinic acid, nicotinic acid ester, or a reduced form thereof.

In some embodiments, the compound of Formula IX is

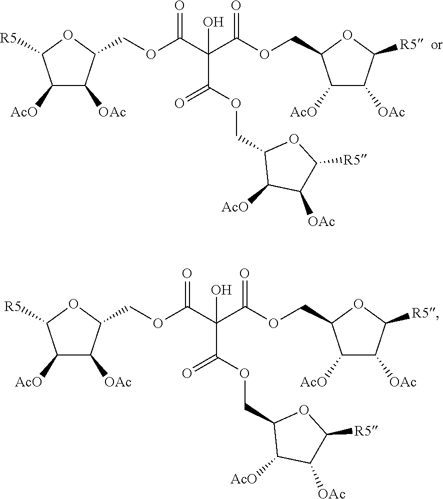

wherein R5, R5′, and R5″ are each independently nicotinamide, dihydronicotinamide, nicotinic acid, nicotinic acid ester, or a reduced form thereof.

Suitably, R1 of Formula V or R3 of Formula IX is an unsubstituted C₁₋₄ alkyl as described herein. In embodiments, the compound of Formula V or Formula IX is any one of

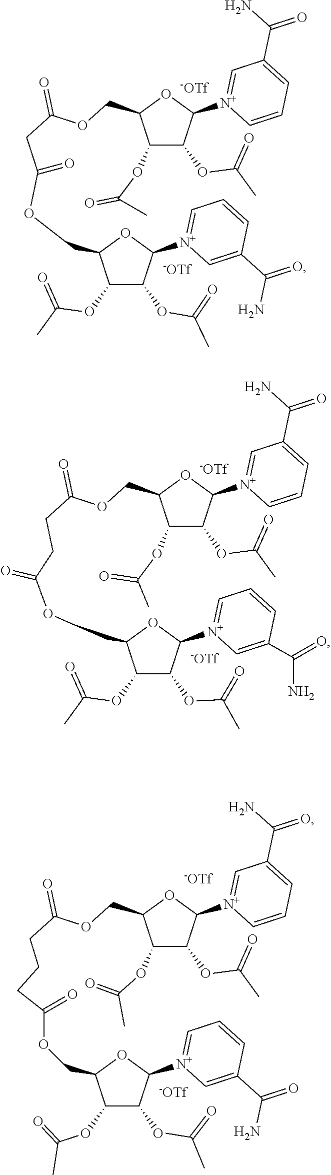

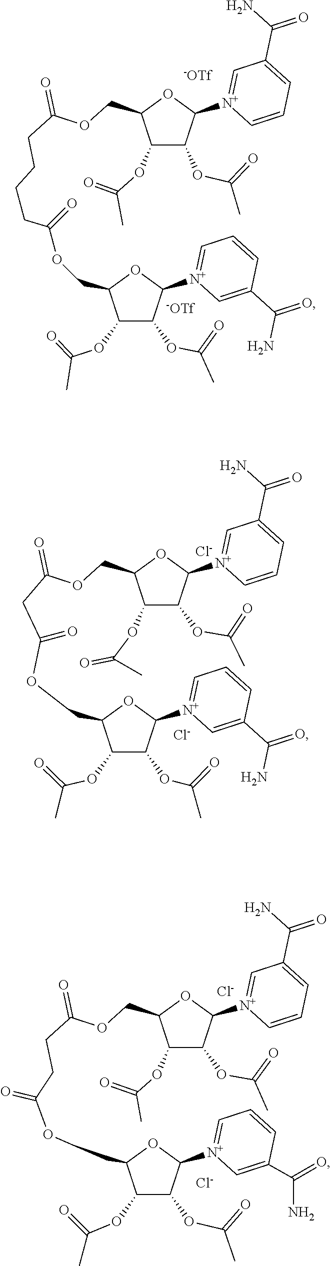

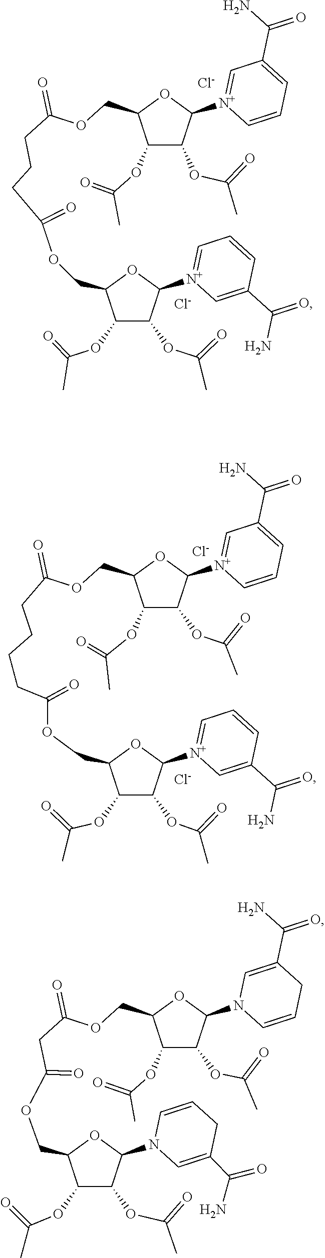

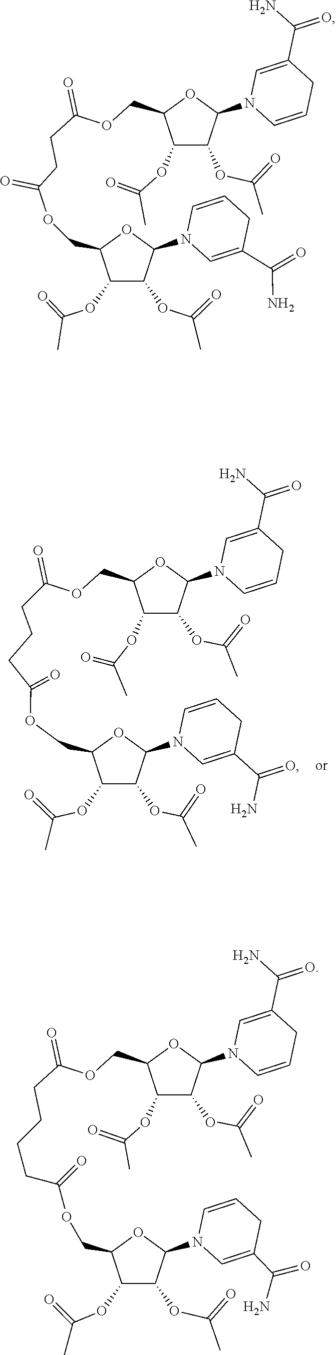 [SMILES: CC(=O)O[C@@H]1[C@H](OC(C)=O)[C@@H](COC(=O)CCC(=O)OC[C@H]2O[C@@H](N3C=CCC(C(N)=O)=C3)[C@H](OC(C)=O)[C@@H]2OC(C)=O)O[C@H]1N1C=CCC(C(N)=O)=C1 ; CC(=O)O[C@@H]1[C@H](OC(C)=O)[C@@H](COC(=O)CCCC(=O)OC[C@H]2O[C@@H](N3C=CCC(C(N)=O)=C3)[C@H](OC(C)=O)[C@@H]2OC(C)=O)O[C@H]1N1C=CCC(C(N)=O)=C1 ; CC(=O)O[C@@H]1[C@H](OC(C)=O)[C@@H](COC(=O)CCCCC(=O)OC[C@H]2O[C@@H](N3C=CCC(C(N)=O)=C3)[C@H](OC(C)=O)[C@@H]2OC(C)=O)O[C@H]1N1C=CCC(C(N)=O)=C1]

Suitably, R1 of Formula V or R3 of Formula IX is a C₂₋₄ alkenyl as described herein. In embodiments, the compound of Formula V or Formula IX is:

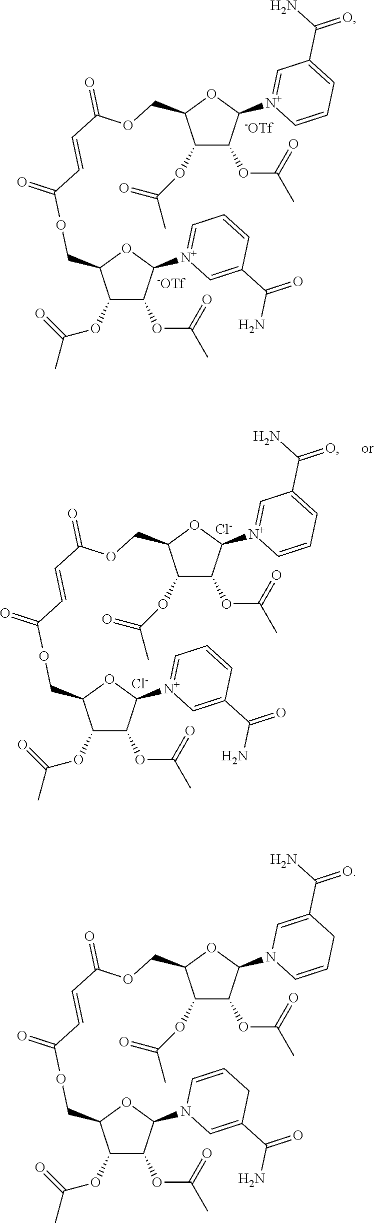

Suitably, R1 of Formula V or R3 of Formula IX is a C₁₋₃ alkyl carboxylate optionally substituted with a protected or a free amine, as described herein. In embodiments, the compound of Formula V or Formula IX is.

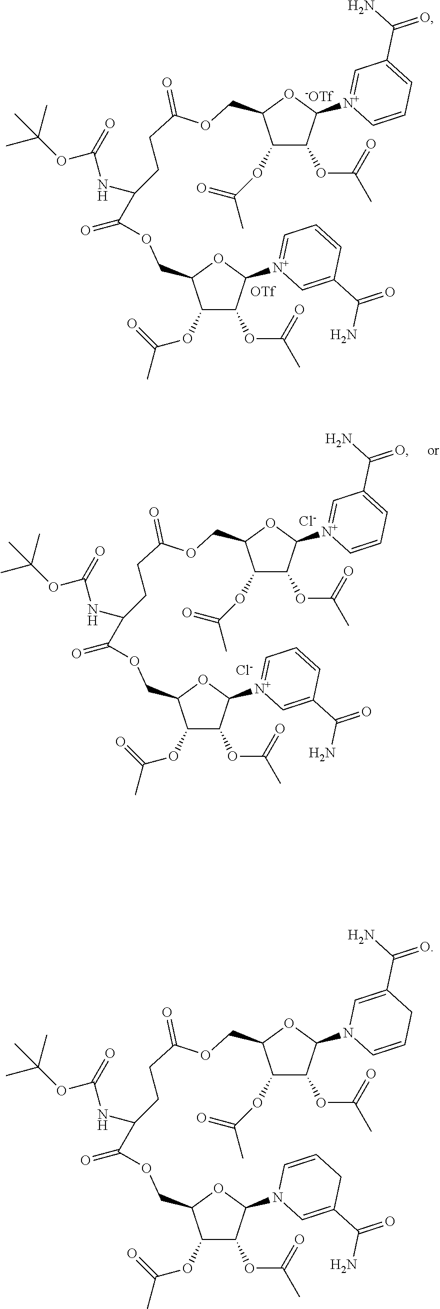

Suitably, R1 of Formula V or R3 of Formula IX is an alkyl PEG ester as described herein, e.g., comprising about 5 to about 200 ethylene glycol units, suitably about 8 to about 150 ethylene glycol units, suitably about 10 to about 100 ethylene glycol units, suitably about 20 to about 80 ethylene glycol units, suitably about 30 to about 70 ethylene glycol units, or suitably about 40 to about 60 ethylene glycol units, or about 10 ethylene glycol units, or about 20 ethylene glycol units, or about 30 ethylene glycol units, or about 40 ethylene glycol units, or about 50 ethylene glycol units, or about 60 ethylene glycol units, or about 70 ethylene glycol units, or about 80 ethylene glycol units, or about 90 ethylene glycol units, or about 100 ethylene glycol units. In exemplary embodiments, the compound of Formula V or Formula IX is:

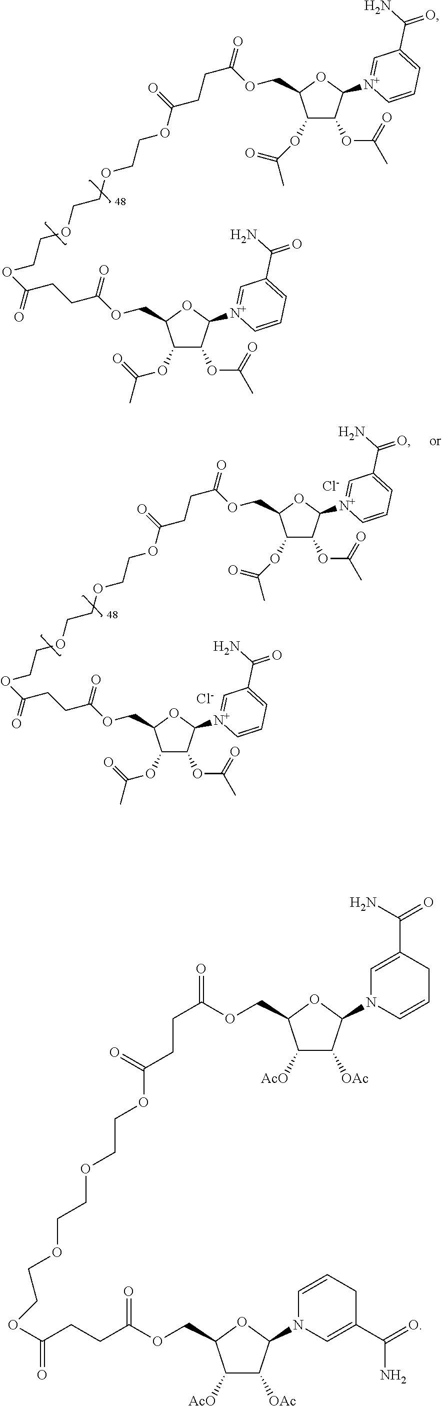

In some embodiments, the compounds of the present disclosure, e.g. the nucleoside and/or nicotinamide ribose conjugates provided herein, are substantially resistant to enzymatic hydrolysis, e.g., by lipases, phosphorylases (such as purine nucleoside phosphorylase (PNP), and the like. As used herein, “substantially resistant to enzymatic hydrolysis” means that the compound does not hydrolyze for at least 15 minutes, at least 30 minutes, at least 1 hour, at least 2 hours, at least 4 hours, at least 6 hours, at least 12 hours, at least 18 hours, or at least 24 hours after being contacted with an enzyme capable of hydrolysis, e.g., a lipase or phosphorylase.

### Methods of Making

In embodiments, the disclosure provides methods of making compounds described herein.

Suitably, a method of making a compound of any one of Formulas I-III or a salt, solvate, or derivative thereof, comprises: (a) adding a protecting group to the 5′-carbon of D-ribose, to form a 5′-protected ribose; (b) acetylating the hydroxy groups at the 1′, 2′, and 3′ carbons of the 5′-protected ribose, to form an acetylated, 5′-protected ribose; (c) deprotecting the 5′-carbon of the acetylated, 5′-protected ribose, to form an acetylated, 5′-deprotected ribose; and (d) coupling the acetylated, 5′-deprotected ribose with a reactant comprising R1, R2, and/or R3, to form the compound of any one of Formulas I-III. In embodiments, the method further comprises (e) reacting the compound of any one of Formulas I-III with a functionalized nucleobase, e.g., a functionalized nicotinamide, to form a compound of any one of Formulas IV-IX.

Protecting groups and their corresponding means deprotection are known to one of ordinary skill in the art. Suitable protecting groups for an alcohol (e.g., the alcohol group at the 5′-carbon of ribose) and their methods of removal include, but are not limited to:
Acetyl (Ac)—Removable by acid or base
Benzoyl (Bz)—Removable by acid or base
Benzyl (Bn)—Removable by hydrogenolysis
β-Methoxyethoxymethyl ether (MEM)—Removable by acid
Dimethoxytrityl, [bis-(4-methoxyphenyl)phenylmethyl](DMT)—Removable by weak acid
Methoxymethyl ether (MOM)—Removable by acid
Methoxytrityl [(4-methoxyphenyl)diphenylmethyl](MMT)—Removable by acid and hydrogenolysis
p-Methoxybenzyl ether (PMB)—Removable by acid, hydrogenolysis, or oxidation (e.g., with DDQ)
p-Methoxyphenyl ether (PMP)—Removable by oxidation
Methylthiomethyl ether—Removable by acid
Pivaloyl (Piv)—Removable by acid, base or reductant agents
Tetrahydropyranyl (THP)—Removable by acid
Tetrahydrofuran (THF)—Removable by acid
Trityl (triphenylmethyl, Tr)—Removable by acid and hydrogenolysis
Silyl ether, such as trimethylsilyl (TMS), tert-butyldimethylsilyl (TBDMS or TBS), tri-iso-propylsilyloxymethyl (TOM), and triisopropylsilyl (TIPS) ethers—Removable by acid or fluoride ion, such as NaF, TBAF (tetra-n-butylammonium fluoride, HF-Py, or HF-NEt3)
Methyl ethers—Removable via cleavage by TMSI in dichloromethane, acetonitrile or chloroform, or by BBr₃ in dichloromethane
Ethoxyethyl ethers (EE)—Removable via cleavage

In some embodiments, the protecting group is a trityl group. Suitably, step (a) of the method comprises contacting D-ribose with trityl chloride to add a trityl group at the 5′-carbon of the ribose ring. Further, step (c) of the method suitably comprises removing the trityl protecting group using an acid, e.g., acetic acid.

Methods for acetylation are known to one of ordinary skill in the art. An exemplary acetylation agent that may be used in step (b) of the method is acetic anhydride (“Ac₂O”). Further suitable acetylation agents include, but are not limited to, acetyl chloride, ketene, thioacetic acid, and the like.

Suitably, following the deprotecting step, the acetylated, 5′-deprotected ribose comprises a hydroxy group at the 5′-carbon (“5′-hydroxy group”). In embodiments, the coupling step of the method comprises contacting the acetylated, 5′-deprotected ribose of step (c) with a reactant that (i) comprises R1, R2, and/or R3 as described herein; and (ii) that is capable of reacting with the 5′-hydroxy group. In some embodiments, the reactant is a monocarboxylic acid of R1, R2, and/or R3. In further embodiments, the reactant is a dicarboxylic acid of R1, R2, and/or R3. In still further embodiments, the reactant is an acid anhydride of R1, R2, and/or R3. In still further embodiments, the reactant is an acid chloride of R1, R2, and/or R3.

In embodiments where the reactant is a monocarboxylic acid or acid chloride of R1, the reactant is coupled with the acetylated, 5′-deprotected ribose to form a compound of Formula I in which one of A or A′ is H. In embodiments where the reactant is a dicarboxylic acid or acid anhydride of R1, the reactant is coupled with the acetylated, 5′-deprotected ribose to form a compound of Formula I in which both of A or A′ are not H.

In a further embodiment, the reactant is a monocarboxylic acid or acid chloride of R2 and is coupled with the acetylated, 5′-deprotected ribose to form a compound of Formula II. In further embodiments, the reactant is a dicarboxylic acid or acid anhydride of R3 and is coupled with the acetylated, 5′-deprotected ribose to form a compound of Formula III.

Suitably, the coupling is performed in the presence of a coupling agent. Coupling reagents, e.g., that converts a hydroxy and a carboxylic acid or an acid anhydride to an ester, are known to one of ordinary skill in the art. In embodiments, the coupling agent is a carbodiimide reagent. Non-limiting examples of carbodiimide reagents include N,N′-dicyclohexylcarbodiimide (DCC), N,N′-diisopropylcarbodiimide (DIC), 1-ethyl-3-(3-dimethylaminopropyl)carbodiimide (EDC, EDAC or EDCI), and 1-cyclohexyl-(2-morpholinoethyl)carbodiimide metho-p-toluene sulfonate (CMCT or CMC). The coupling reaction may also include a catalyst, e.g., 4-dimethylaminopyridine (DMAP). In certain embodiments, the acetylated, 5′-deprotected ribose and the reactant comprising R1, R2, and/or R3 is coupled in the presence of DCC or EDC and DMAP.

In embodiments, the methods herein comprise batch and semi-continuous processes that enable the production of compounds of any one of Formulas I-IX a salt, solvate, or derivative thereof, wherein the use of solvents is kept to a minimum, and wherein conversion and reaction times are optimized by the use of sealed conditions, continuous liquid-liquid extraction, and/or mechanochemistry, and an optimized purification procedure.

As used herein, the term “solvent” refers to a compound or mixture of compounds including, but not limited to, water, water in which an ionic compound has been dissolved, acetic acid, acetone, acetonitrile, benzene, 1-butanol, 2-butanol, t-butyl alcohol (“TBA”), 2-butanone, carbon tetrachloride, chlorobenzene, chloroform, cyclohexane, 1,2-dichloroethane (“DCE”), diethylene glycol, diethyl ether (“Et2O”), diglyme (diethylene glycol dimethyl ether), 1,2-dimethoxyethane (“DME”), N,N-dimethylformamide (“DMF”), dimethylsulfoxide (“DMSO”), 1,4-dioxane, ethanol, ethyl acetate (“EtOAc”), ethylene glycol, glycerin, heptanes, hexamethylphosphoramide (“HMPA”), hexamethylphosphorus triamide (“HMPT”), hexane, methanol (“MeOH”), methyl t-butyl ether (“MTBE”), methylene chloride (“DCM,” “CH2Cl2”), N-methyl-2-pyrrolidinone (“NMP”), nitromethane, pentane, petroleum ether, 1-propanol (“n-propanol,” “n-PrOH”), 2-propanol (“isopropanol,” “iPrOH”), pyridine, tetrahydrofuran (“THF”), toluene, triethylamine (“TEA,” “Et3N”), o-xylene, m-xylene, and/or p-xylene, and the like. Solvent classes may include hydrocarbon, aromatic, aprotic, polar, alcoholic, and mixtures thereof.

As used herein, “mechano-chemical mixing,” “mechanochemistry,” and “mechanical processing” refer to techniques known to those of ordinary skill in the art, in which chemical starting materials and/or reagents with disparate solubility properties are reacted, for example, by direct milling, liquid assisted-milling, triturating, mixing, or grinding, generally in the absence of solvents. Interchangeable terms may include “mechanic-chemical,” or the like. See, e.g., Ravalico et al., “Rapid synthesis of nucleotide pyrophosphate linkages in a ball mill,” Org Biol Chem 9:6496 (2011); Hasa et al., “Cocrystal Formation through Mechanochemistry: From Neat and Liquid-Assisted Grinding to Polymer-Assisted Grinding,” Angewandte Chemie 127:7371 (2015); Crossey et al., “Atom efficient synthesis of pyrimidine and purine nucleosides by ball milling,” RSC Adv 5:58116 (2015); and Johnston et al., “Applications of Mechanochemistry for the Synthesis of DNA on Ionic Liquid Supports,” Chemistry—Methods 1:1-8 (2021). Mechanochemistry is further described, e.g., in U.S. Pat. No. 9,975,915. The disclosures of each of these references are hereby incorporated by reference in their entireties for all purposes, and specifically with regard to methods of “mechano-chemical mixing,” “mechanochemistry,” and “mechanical processing.”

As used herein, the term “liquid-assisted mixing” refers to a technique known to those of ordinary skill in the art, in which the kinetics of solid-state grinding is accelerated by addition of a small amount of liquid during mixing. It was discovered that not only did small amounts of liquid speed up the solid-state reaction, but in numerous cases, addition of small amounts of liquid allowed the formation of new solid forms that could not otherwise be made. See, e.g., Shan et al., “Mechanochemistry and co-crystal formation: effect of solvent on reaction kinetics,” Chem Comm 2002:2732-2373 (2002). It was further discovered that the exact outcome of the solid-state grinding could be controlled by careful choice of the added liquid. See, e.g., Trask et al., “Achieving Polymorphic and Stoichiometric Diversity in Cocrystal Formation: Importance of Solid-State Grinding, Powder X-ray Structure Differentiation, and Seeding,” Crystal Growth & Design 5:2233 (2005). It was further demonstrated that this liquid-assisted mixing approach is significantly more effective in searching for alternate solid forms of drug candidates than other previously used methods, e.g., conventional solution crystallization or melt growth. See, e.g., Karki et al., “Screening for pharmaceutical cocrystal hydrates via neat and liquid-assisted grinding,” Molecular Pharmaceutics 4:347 (2007); and Trask et al., “Screening for crystalline salts via mechanochemistry,” Chem Comm 2006:51 (2006). Liquid-assisted mixing is a method that is rapid and environmentally friendly because it eliminates the need to use large amounts of solvents, reducing waste and increasing cost efficiency.

In embodiments, the coupling reaction is performed via a mechanochemical reaction, e.g., in a ball milling jar. Ball milling conditions may be selected by one of ordinary skill in the art, e.g., milled for about 10 minutes to about 60 minutes, or about 15 minutes to about 40 minutes, or about 20 minutes to about 30 minutes, or about 10, about 15, about 20, about 25, about 30, about 35, about 40, about 45, about 50, about 55, about 60, or more than 60 minutes, at a frequency of about 10 Hz to about 60 Hz, or about 20 Hz to about 50 Hz, or about 30 Hz to about 40 Hz, or about 10, about 15, about 20, about 25, about 30, about 35, about 40, about 45, about 50, about 55, or about 60 Hz. In some embodiments, the ball milling is performed for about 20 to about 40 minutes, or about 20 to about 30 minutes at about 30 Hz.

One of ordinary skill in the art would appreciate that, in addition to mechanochemistry techniques, conventional solution chemistry may be employed in the methods for producing the compounds described herein, any one of Formulas I to IX.

In some embodiments, the compound of any one of Formulas I to III is contacted with a functionalized nucleobase to form the corresponding compound of any one of Formulas IV to VI. In further embodiments, the compound of any one of Formulas I to III is contacted with a functionalized nicotinamide to form the corresponding compound of any one of Formulas VII to IX.

The functionalized nucleobase or functionalized nicotinamide comprises a functional group that is capable of reacting with an ester, e.g., the ester at the 1′ ribose carbon in any one of Formulas I to III. Suitably, the functional group is linked to the amide nitrogen of nicotinamide. Suitably, the functionalized nucleobase or functionalized nicotinamide comprises a trialkylsilyl group, e.g., a trimethylsilyl group. In exemplary embodiments, the functionalized nicotinamide is N-trimethylsilyl-nicotinamide, also known as nicotinamide TMS:

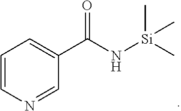

Suitably, the functionalized nucleobase or functionalized nicotinamide, e.g., comprising a trialkylsilyl group, is coupled to the compound of Formulas I to III in the presence of a catalyst. In embodiments, the catalyst comprises a Lewis acid, such as trimethylsilyl trifluoromethanesulfonate (“TMSOTf”). In some embodiments, the coupling is performed via a mechanochemical reaction, e.g., in a ball milling jar with minimal amount of solvent (e.g., anhydrous dichloromethane). Mechanochemistry and ball milling are described herein. In some embodiments, the compound of any one of Formulas I to III is mixed with the functionalized nucleobase or functionalized nicotinamide and subjected to ball milling for about 10 minutes to about 60 minutes, or about 15 minutes to about 40 minutes, or about 20 minutes to about 30 minutes, or about 10, about 15, about 20, about 25, about 30, about 35, about 40, about 45, about 50, about 55, about 60, or more than 60 minutes, at a frequency of about 10 Hz to about 60 Hz, or about 20 Hz to about 50 Hz, or about 30 Hz to about 40 Hz, or about 10, about 15, about 20, about 25, about 30, about 35, about 40, about 45, about 50, about 55, or about 60 Hz. In some embodiments, the ball milling is performed for about 20 to about 40 minutes, or about 20 to about 30 minutes at about 30 Hz. The resulting product may be a compound of any one of Formulas IV to IX. In embodiments where the compound of any one of Formulas I to III is mixed with nicotinamide TMS, the resulting product is the corresponding compound of any one of Formulas VII to IX, wherein R5 is nicotinamide, and the compound comprises a triflate counteranion.

In some embodiments, the nicotinamide of any one of Formulas VII to IX is reduced to dihydronicotinamide, e.g., using a reducing agent such as sodium dithionite (Na₂S₂O₄), thereby providing a compound of Formulas VII to IX, wherein R5 is dihydronicotinamide. A further example of a suitable reducing agents is sodium borohydride (NaBH₄).

In further embodiments, the nicotinamide with triflate counteranion of any one of Formulas VII to IX is subjected to ion exchange for a different counteranion, e.g., a chloride ion. Additional non-limiting examples of counteranions are described herein. The ion exchange may be performed using any suitable ion exchange resin, e.g., bromide resins that may be functionalized to include the desired counter anion to be exchanged; and AmberLite™ resin available from DuPont.

An exemplary method of making the compound of Formula III, in which R3 is an unsubstituted C₁₋₄ alkyl as described herein (depicted below as compounds 6a-h), is as follows:

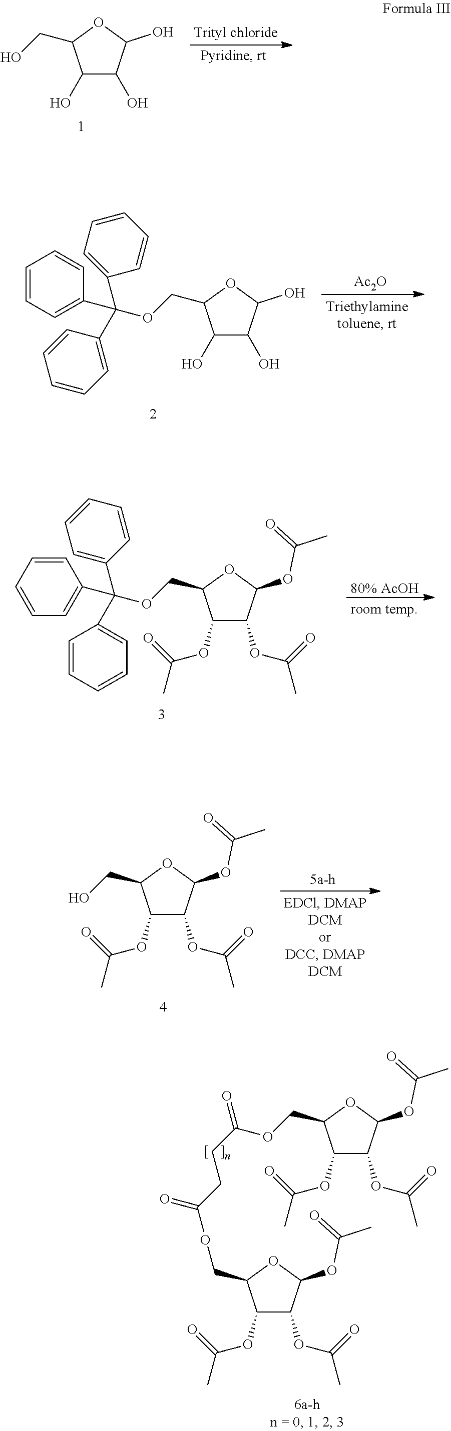

Suitably, compounds 6a-h are used to prepare compounds of Formula IX, in which R3 is an unsubstituted C₁₋₄ alkyl as described herein (depicted below as compounds 10a-h), as follows:

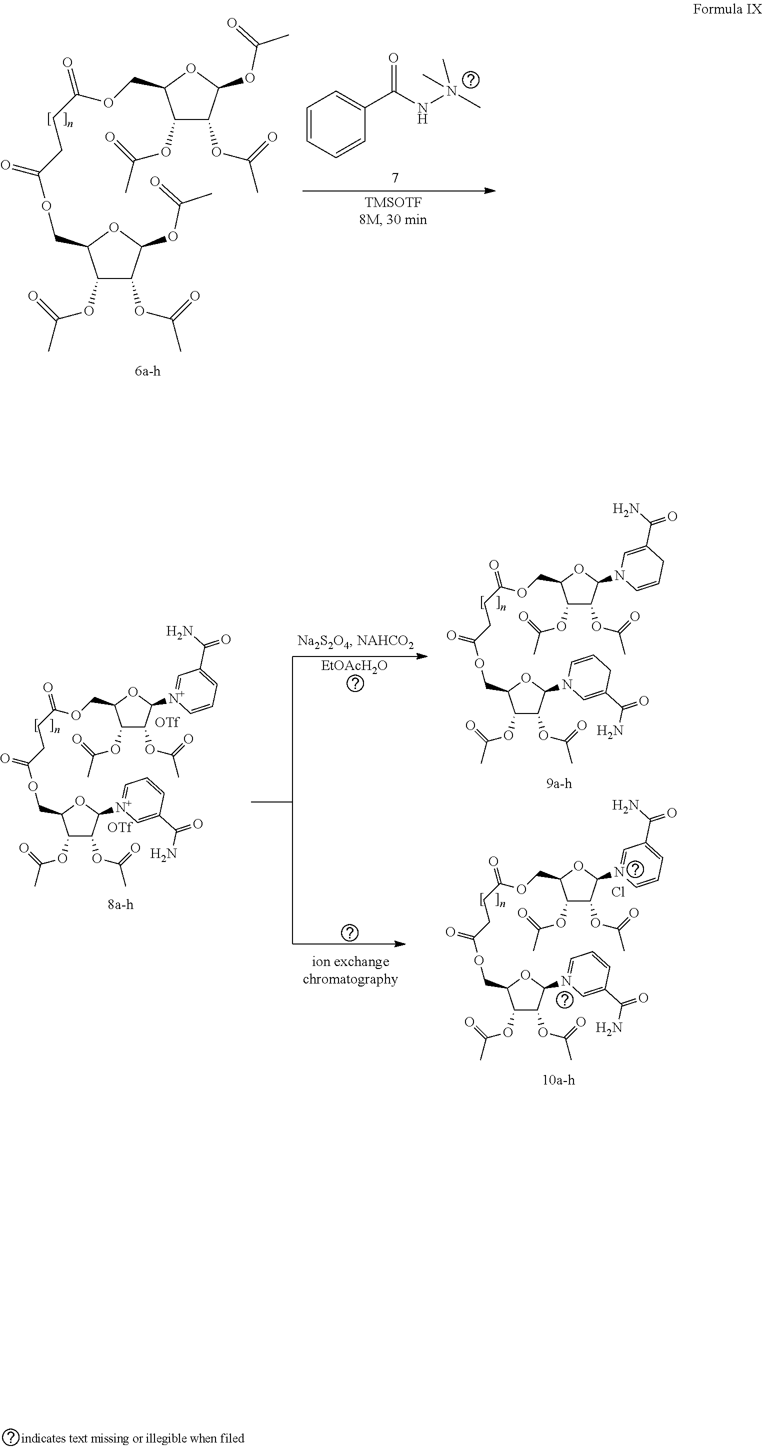

An exemplary method of making the compound of Formula II, which R2 is an alkyl PEG ester as described herein (depicted below as compound 6i), as follows:

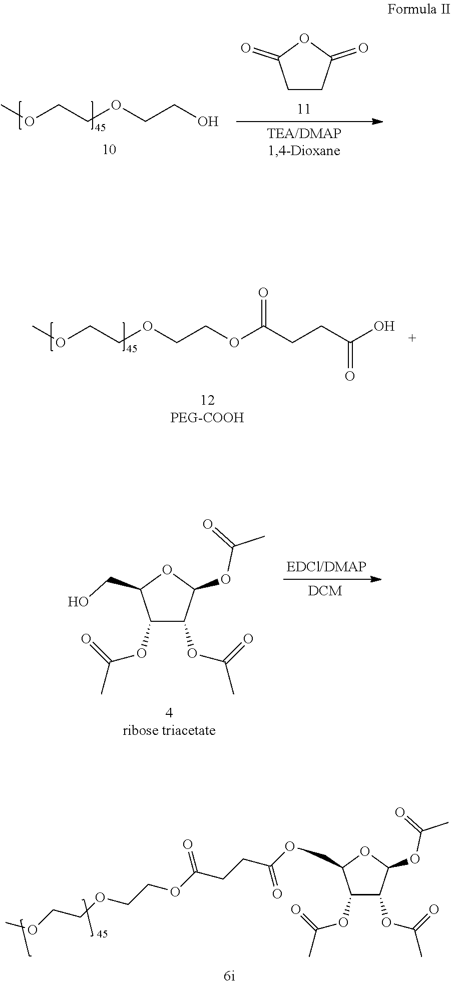

Suitably compound 6i is used to prepare compound of Formula VII, in which R2 is an alkyl PEG ester as described herein (depicted below as compound 8i), as follows:

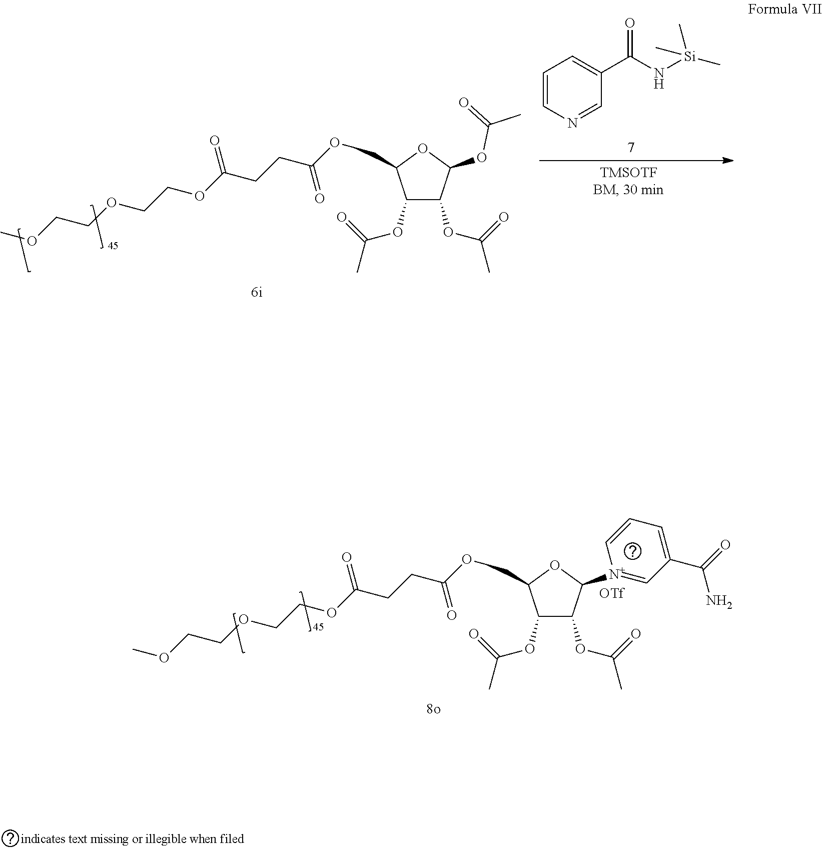

An exemplary method of making the compound of Formula III, in which R3 is an alkyl PEG ester as described herein (depicted below as compound 17), is as follows:

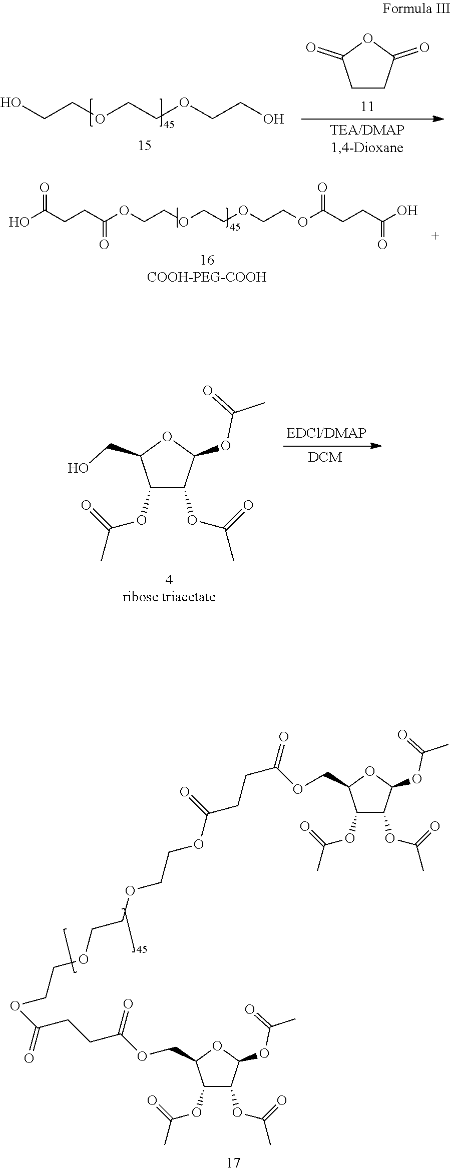

Suitably, compound 17 is used to prepare a compound of Formula IX, in which R3 is an alkyl PEG ester as described herein (depicted below as compound 18), as follows:

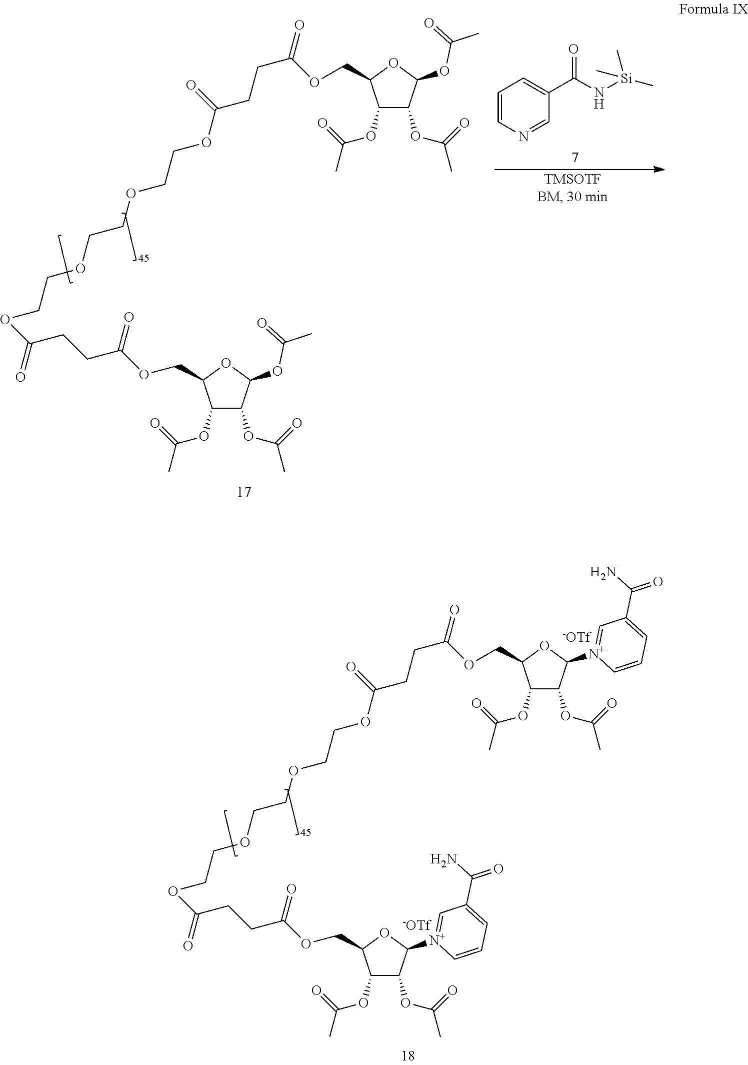

### Compositions and Methods of Use

In embodiments, the compounds provided herein, e.g., a compound of any one of Formulas I to IX, preferably a compound of any one of Formulas IV to IX, more preferably a compound of any one of Formulas VII to IX, may be included in a composition. In exemplary embodiments, provided herein is an NAD-increasing composition that may be administered to a subject in need thereof. Suitably, the NAD-increasing composition comprises a compound of Formula VII, Formula VIII, Formula IX, or combination thereof.

In exemplary embodiments, the composition is an oral formulation, including a liquid, drops, a spray, a solution, a gel, a powder, a suspension, or in a solid dosage form such as a lozenge, a capsule, a tablet, a pill, a gel-cap, a buccal or sub-lingual strip, and the like.

In embodiments, the composition comprises a compound of any one or more of Formulas VII to IX at about 100 mg to about 1000 mg, or about 200 mg to about 800 mg, or about 300 mg to about 700 mg, or about 400 to about 600 mg, or about 100 mg, about 200 mg, about 300 mg, about 400 mg, about 500 mg, about 600 mg, about 700 mg, about 800 mg, about 900 mg, or about 1000 mg. In embodiments, the composition comprises an equivalent amount of nicotinamide riboside (NR) of about 100 mg to about 1000 mg, or about 200 mg to about 800 mg, or about 300 mg to about 700 mg, or about 400 to about 600 mg, or about 100 mg, about 200 mg, about 300 mg, about 400 mg, about 500 mg, about 600 mg, about 700 mg, about 800 mg, about 900 mg, or about 1000 mg. For example, one molar equivalent of a compound of Formula IX provides two or three molar equivalents of NR, and thus, a formulation intended to provide 500 mg of NR may suitably comprise 167 mg or 250 mg of the compound of Formula IX.

In embodiments, the composition further comprises one or more of: i) a sirtuin activating compound; ii) a CD38 inhibiting compound; and iii) a poly ADP ribose polymerase (PARP) inhibiting compound.

As used herein, a “sirtuin activating compound” or STAC refers to a chemical compound that activates sirtuins, a group of enzymes that use NAD⁺ to remove acetyl groups from proteins. Examples of sirtuin activating compounds include polyphenols such as resveratrol, butein, piceatannol, isoliquiritigenin, fisetin, and quercetin. Amounts of sirtuin activating compounds are suitably included in the compositions described herein in an amount of about 25 mg to about 1000 mg, about 100 mg to about 1000 mg, about 25 mg to about 500 mg, about 25 mg to about 200 mg, about 25 mg to about 250 mg, about 30 mg to about 225 mg, about 40 mg to about 200 mg, about 45 mg to about 250 mg, or about 25 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg.

As used herein, a “CD38 inhibiting compound” refers to a compound that inhibits the NAD⁺ase CD38. Examples of CD38 inhibiting compounds include flavonoids, including quercetin, apigenin, and the like. Amounts of CD38 inhibiting compounds are suitably included in the compositions described herein in an amount of about 25 mg to about 1000 mg, about 100 mg to about 1000 mg, about 25 mg to about 500 mg, about 25 mg to about 200 mg, about 25 mg to about 250 mg, about 30 mg to about 225 mg, about 40 mg to about 200 mg, about 45 mg to about 250 mg, or about 25 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg.

As used herein, a “poly ADP ribose polymerase (PARP) inhibiting compound” refers to a compound that inhibits poly ADP ribose polymerase, a family of proteins involved in a number of cellular processes such as DNA repair, genomic stability, and programmed cell death. PARP family proteins include PARP1, PARP2, VPARP (PARP4), Tankyrase-1 and -2 (PARP-5a or TNKS, and PARP-5b or TNKS2). Others include PARP3, PARP6, TIPARP (or “PARP7”), PARP8, PARP9, PARP10, PARP11, PARP12, PARP14, PARP15, and PARP16. Examples of PARP inhibiting compounds include Olaparib, Rucaparib and Niraparib, and the like. Amounts of PARP inhibiting compounds are suitably included in the compositions described herein in an amount of about 25 mg to about 1000 mg, about 100 mg to about 1000 mg, about 25 mg to about 500 mg, about 25 mg to about 200 mg, about 25 mg to about 250 mg, about 30 mg to about 225 mg, about 40 mg to about 200 mg, about 45 mg to about 250 mg, or about 25 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg.

Suitably, the composition also comprises pterostilbene. Pterostilbene is a polyphenol based derivative of resveratrol and, like the NAD⁺ precursor, promotes metabolic health. The chemical structure of pterostilbene is provided below:

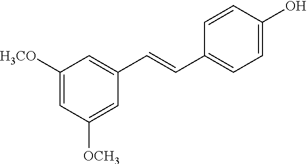 [SMILES: COc1cc(/C=C/c2ccc(O)cc2)cc(OC)c1]

In some embodiments, a derivative, salt, solvate, or prodrug of pterostilbene can be used in the compositions described herein. In certain embodiments, pterostilbene may be substituted and/or combined with epsilon-viniferin and/or resveratrol.

As described herein, the compositions for use in treatment are suitably formulated for oral delivery, i.e., in an oral formulation. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc., or into liposomes. Such compositions may influence the physical state, stability, rate of in vivo release, and rate of in vivo clearance of the disclosed. See, e.g., Remington's Pharmaceutical Sciences, 18th Ed. 1990 (Mack Publishing Co., Easton, Pa. 18042), pp. 1435-1712. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also, Marshall, K. In: Modern Pharmaceutics Edited by G. S. Banker and C. T. Rhodes Chapter 10, 1979. The formulation may include a peptide (or chemically modified forms thereof) and inert ingredients which protect compounds in the stomach environment, and release of the biologically active material in the intestine.

The compounds described herein, e.g., of Formula VII, Formula VIII, and/or Formula IX, pterostilbene, nicotinamide mononucleotide, niacin, epsilon-viniferin, and/or resveratrol or derivatives thereof may be chemically modified so that oral delivery of the compound is efficacious. Contemplated chemical modification is the attachment of at least one moiety to the component molecule itself, where the moiety permits uptake into the blood stream from the stomach or intestine, or uptake directly into the intestinal mucosa. Also contemplated is the increase in overall stability of the component or components and increase in circulation time in the body. Certain embodiments may be pharmaceutical compositions. Certain embodiments may be nutritional supplements.

Certain embodiments provide liquid dosage forms for oral administration, including pharmaceutically acceptable emulsions, solutions, suspensions, and syrups, which may contain other components including inert diluents, adjuvants such as wetting agents, emulsifying and suspending agents, and sweetening, and flavoring agents.

Controlled release oral formulations may be provided. Controlled release may include, but is not limited to, delayed release and pH-dependent release. In certain embodiments, the compound of Formula VII, Formula VIII, and/or Formula IX, or derivatives thereof can be incorporated into microcapsules, microparticulates, nanoparticulates, and the like through use of coatings to affect release of the active principle. In certain embodiments, the compound of Formula VII, Formula VIII, and/or Formula IX, or derivatives thereof can be incorporated into an inert matrix which permits release by either diffusion or leaching mechanisms, e.g., gums. Slowly degenerating matrices may also be incorporated into the formulation.

Modified release oral formulations may be provided. Modified release may allow for specific release profiles. Extended release oral formulations may be provided. Extended release may allow for release of active ingredient over a desired time period. Additional discussions for varying release formulations and related terms may be found in Lesczek Krowczynski, Extended-Release Dosage Forms, 1987 (CRC Press, Inc.).

In certain aspects, the form of a controlled, modified or extended release oral formulation is a tablet, capsule, or microbeads for oral administration. In other aspects, controlled, modified or extended release formulations comprising suitable and effective treatment amounts of the desired components may be pills, powders, granules, sterile parenteral solutions or suspensions, oral solutions or suspensions, oil water emulsions as well as implants and microencapsulated delivery systems.

Other formulations may provide controlled, modified or extended release profiles. Compositions of the present invention may comprise conventional pharmaceutical binders, excipients and additives, which may act to control, modify or extend release when used in sufficient quantities. Coating agents, e.g., plasticizers, may be used to enhance the controlled, modified or extended release features of the compositions of the invention.

For oral formulations, the location of release may be the stomach, the small intestine (the duodenum, the jejunum, or the ileum), or the large intestine. The release can avoid the deleterious effects of the stomach environment, either by protection of the agent (or derivative) or by release of the agent (or derivative) beyond the stomach environment, such as in the intestine. To ensure full gastric resistance, a coating temporally impermeable to at least pH 5.0 is useful. Examples of the more common inert ingredients that are used as enteric coatings are cellulose acetate trimellitate (CAT), hydroxypropylmethylcellulose phthalate (HPMCP), polyvinyl acetate phthalate (PVAP), poly(methacrylic acid-co-ethyl acrylate) 1:1, cellulose acetate phthalate (CAP), poly(methacylic acid-co-methyl methacrylate) 1:1, poly(methacylic acid-co-methyl methacrylate) 1:2, and natural shellac resin. These coatings may be used as mixed films.

In exemplary embodiments, the compositions may be provided in soft capsules. The soft capsule can be prepared using techniques known to one of skill in the art. For example, soft capsules are typically produced using a rotary die encapsulation process. Active agent formulations are fed into the encapsulation machine by gravity. In an embodiment, the formulation comprises pharmaceutical excipients such as olive oil, gelatin, glycerin, purified water, beeswax yellow, sunflower lecithin, silicon dioxide, titanium dioxide, a colorant, microcrystalline cellulose, hypromellose, vegetable magnesium stearate, and/or silica.

A capsule shell can comprise one or more plasticizers such as glycerin, sorbitol, sorbitans, maltitol, glycerol, polyethylene glycol, polyalcohols with 3 to 6 carbon atoms, citric acid, citric acid esters, triethyl citrate and combinations thereof. In an embodiment, the plasticizer is glycerin.

In addition to the plasticizer(s), the capsule shell can include other suitable shell additives such as opacifiers, colorants, humectants, preservatives, flavorings, and buffering salts and acids.

Opacifiers are used to opacify the capsule shell when the encapsulated active agents are light sensitive. Suitable opacifiers include, but not limited to, titanium dioxide, zinc oxide, calcium carbonate and combinations thereof. In an embodiment, the opacifier is titanium dioxide.

Colorants can be used to for marketing and product identification and/or differentiation purposes. Suitable colorants include synthetic and natural dyes and combinations thereof.

Humectants can be used to suppress the water activity of a soft gel capsule. Suitable humectants include glycerin and sorbitol, which are often components of the plasticizer composition. Due to the low water activity of dried, properly stored soft gel capsules, the greatest risk from microorganisms comes from molds and yeasts. Preservatives can be incorporated into the capsule shell. Suitable preservatives include alkyl esters of p-hydroxy benzoic acid such as methyl, ethyl, propyl, butyl and heptyl (collectively known as “parabens”) or combinations thereof.

In embodiments, the compounds described herein are administered to a subject in need thereof, e.g., via a composition described herein. In embodiments, a compound of Formula VII, Formula VIII, Formula IX, or combination thereof, is administered to a subject at an amount of about 50 mg to about 1500 mg, about 100 mg to about 1500 mg, about 100 mg to about 1000 mg, about 125 mg to about 900 mg, about 150 mg to about 850 mg, about 200 mg to 700 mg, about 200 mg to about 500 mg, about 1000 mg to about 1500 mg, or about 100 mg, about 150 mg, about 200 mg, about 250 mg, about 300 mg, about 350 mg, about 400 mg, about 450 mg, about 500 mg, about 550 mg, about 600 mg, about 650 mg or about 700 mg. Suitably, these amounts are administered to a subject on a daily basis in the form of a single composition or multiple compositions. One of ordinary skill in the art would understand how to adjust the amounts of compounds based on the amount of nicotinamide intended to be administered. For example, in embodiments where a single molar equivalent of the compound comprises two molar equivalents of NR (e.g., a compound of Formula IX), the amount of the compound to be administered is reduced by half. In embodiments, pterostilbene or derivative thereof is further administered to the subject, e.g., at an amount of about 25 mg to about 1000 mg, about 100 mg to about 1000 mg, about 25 mg to about 500 mg, about 25 mg to about 200 mg, about 25 mg to about 250 mg, about 30 mg to about 225 mg, about 40 mg to about 200 mg, about 45 mg to about 250 mg, or about 25 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg. Suitably, these amounts are administered to a patient on a daily basis in the form of a single composition or multiple compositions.

In additional exemplary embodiments, the compounds and/or compositions described herein are provided as oral formulations, topical formulations, injectable or infusion formulations, inhalable or sprayable formulations. In embodiments, various components of the composition (e.g., the compound of Formula VII, Formula VIII, and/or Formula IX; pterostilbene; and/or additional components described herein) are provided in the same composition. In embodiments, the components of the composition are provided in separate compositions and are co-administered at the same time or administered at different time points.

Suitably, methods of treatment or prevention described herein comprise administering, to a subject in need thereof, a composition comprising a combination of a compound provided herein, e.g., a compound of Formula VII, Formula VIII, and/or Formula IX, at about 200 mg to about 700 mg; and pterostilbene at about 25 mg to about 200 mg. In embodiments, the composition comprises about 500 mg of a compound provided herein, e.g., a compound of Formula VII, Formula VIII, and/or Formula IX; and about 100 mg pterostilbene. In embodiments, the composition comprises about 250 mg of a compound provided herein, e.g., a compound of Formula VII, Formula VIII, and/or Formula IX; and about 50 mg pterostilbene. In embodiments, the compositions are administered daily, twice-daily, once every two days, once every three days, or once per week. The administration can be via any administration route described herein.

The methods can involve the use of a composition described herein that is administered as a liquid with an active agent (i.e., a compound of Formula VII, Formula VIII, and/or Formula IX) dissolved (e.g., in solution) or dispersed (e.g., in suspension) in the composition. The solution or suspension may be prepared using one or more pharmaceutically acceptable excipients. Pharmaceutically acceptable excipients are described herein and include, but are not limited to, surfactants, humectants, plasticizers, crystallization inhibitors, wetting agents, bulk filling agents, solubilizers, bioavailability enhancers, pH adjusting agents, flavorants, and combinations thereof.

In embodiments, a composition described herein is administered in a dosage regimen over days, weeks, or months. Dosages may be multiple times per day or singular doses per day. Each dosage when dosages are administered over multiple days, weeks, or months may not be equal amounts. Dosage amounts during a dosage regimen may vary according to the amounts and ranges disclosed herein. Suitably, the compositions described herein are administered on a daily basis for a period of at least 4 weeks, suitably for a period of at least 1 month, or for a period of at least 2 months, at least 3 months, at least 4 months, at least 5 months, or at least 6 months. The compositions described herein can also be administered to a patient for 1 or more years, including for the lifetime of a patient.

All references cited herein, including patents, patent applications, papers, textbooks and the like, and the references cited therein, to the extent that they are not already, are hereby incorporated herein by reference in their entirety.

### Exemplary Embodiments

Embodiment 1. A compound of Formula I:

A-R1-A′ [Formula I]

wherein A and A′ are independently H or

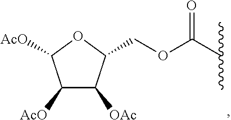

wherein A and A′ are not both H, and:
when one of A or A′ is H, R1 is a substituted or unsubstituted C₈₋₂₄ alkyl, C₈₋₂₄ alkenyl, aryl, alkylaryl, or alkenylaryl; or
when A and A′ are both not H, R1 is a substituted or unsubstituted C₁₋₄ alkyl, C₂₋₄ alkenyl, alkyl polyethylene glycol (PEG) ester, or C₁₋₃ alkyl carboxylate optionally substituted with a protected or free amine; and Ac is an acetyl group.
   Embodiment 2. The compound of embodiment 1, wherein R1 is an alkyl PEG ester.
   Embodiment 3. The compound of embodiment 2, wherein the PEG ester alkyl comprises 10 to 100 ethylene glycol units.
   Embodiment 4. A compound of Formula II:

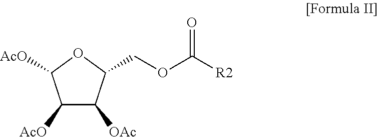

wherein R2 is a substituted or unsubstituted C₈₋₂₄ alkyl, C₈₋₂₄ alkenyl, aryl, alkylaryl, alkenylaryl, or PEG ester alkyl, and
Ac is an acetyl group.
Embodiment 5. The compound of embodiment 4, wherein R2 is a C₁₂ alkyl, a C₁₈ alkenyl, a C₂₀ alkenyl, or a C₂₂ alkenyl.
Embodiment 6. The compound of embodiment 5, wherein

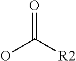

is a substituted or unsubstituted palmitoyl, oleoyl, linoleoyl, linolenoyl, arachidonoyl eicosapentaenoyl, or docosahexaenoyl group.
Embodiment 7. The compound of embodiment 4, wherein

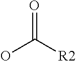

is a succinate ester of retinol.
Embodiment 8. The compound of embodiment 4, wherein

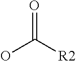

is an omega-3 fatty acid ester.
Embodiment 9. The compound of embodiment 4, wherein R2 is a substituted or unsubstituted phenyl or benzyl.
Embodiment 10. The compound of embodiment 9, wherein R2 is a benzyl that comprises an amino substituent.
Embodiment 11. The compound of embodiment 9, wherein R2 is a para-aminobenzyl.
Embodiment 12. The compound of embodiment 4, wherein R2 is a mycophenolate.
Embodiment 13. The compound of embodiment 4, wherein R2 is an alkyl PEG ester.
Embodiment 14. The compound of embodiment 13, wherein the alkyl PEG ester comprises 10 to 100 ethylene glycol units.
Embodiment 15. A compound of Formula III:

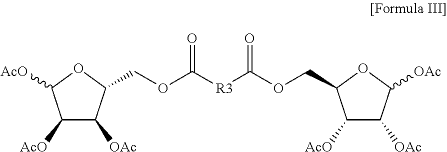

wherein R3 is a substituted or unsubstituted C₁₋₄ alkyl, C₂₋₄ alkenyl, or C₁₋₃ alkyl carboxylate optionally substituted with a protected or a free amine,
or wherein R3 comprises a carbon bonded to (i) a hydroxyl group and (ii) one of

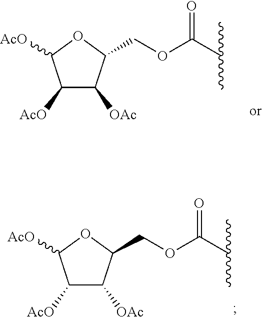 [SMILES: *C(=O)OC[C@H]1OC(OC(C)=O)[C@H](OC(C)=O)[C@@H]1OC(C)=O ; *C(=O)OC[C@@H]1OC(OC(C)=O)[C@@H](OC(C)=O)[C@H]1OC(C)=O]

and
Ac is an acetyl group.
Embodiment 16. The compound of embodiment 12, wherein the compound of Formula III is any one of

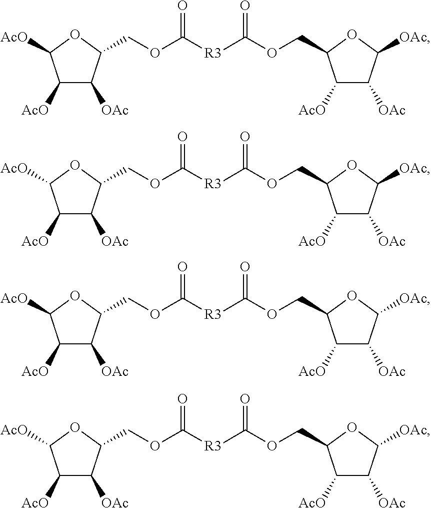

or a mixture thereof.
Embodiment 17. The compound of embodiment 12 or 13, wherein R3 is an unsubstituted C₁₋₄ alkyl.
Embodiment 18. The compound of embodiment 12 or 13, wherein R3 is C₂ alkenyl.
Embodiment 19. The compound of embodiment 12 or 13, wherein R3 is a C₁₋₃ alkyl substituted with a Boc-protected amine.
Embodiment 20. The compound of embodiment 12 or 13, wherein R3 is a C₁₋₃ alkyl substituted with an N-Boc protected glutamate or aspartate.
Embodiment 21. The compound of embodiment 12 or 13, wherein R3 is a C₃ alkyl substituted with

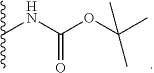 [SMILES: *NC(=O)OC(C)(C)C]

Embodiment 22. The compound of embodiment 15, wherein the compound of Formula III is:

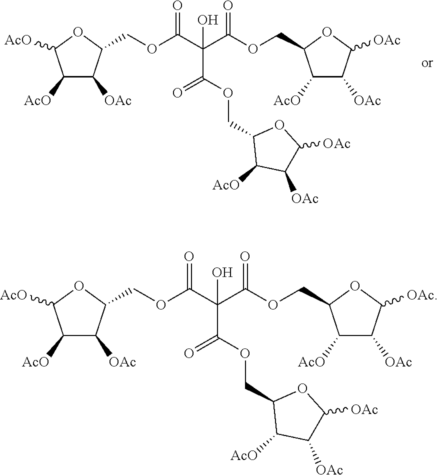 [SMILES: CC(=O)OC1O[C@@H](COC(=O)C(O)(C(=O)OC[C@H]2OC(OC(C)=O)[C@H](OC(C)=O)[C@@H]2OC(C)=O)C(=O)OC[C@H]2OC(OC(C)=O)[C@H](OC(C)=O)[C@@H]2OC(C)=O)[C@H](OC(C)=O)[C@@H]1OC(C)=O ; CC(=O)OC1O[C@H](COC(=O)C(O)(C(=O)OC[C@H]2OC(OC(C)=O)[C@H](OC(C)=O)[C@@H]2OC(C)=O)C(=O)OC[C@H]2OC(OC(C)=O)[C@H](OC(C)=O)[C@@H]2OC(C)=O)[C@@H](OC(C)=O)[C@H]1OC(C)=O]

Embodiment 23. A compound of Formula IV:

B—R1-B′ [Formula IV]

wherein B and B′ are independently H or

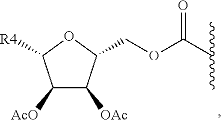

wherein B and B′ are not both H, and:
when one of B or B′ is H, R1 is a substituted or unsubstituted C₈₋₂₄ alkyl, C₈₋₂₄ alkenyl, aryl, alkylaryl, or alkenylaryl; or
when B and B′ are both not H, R1 is a substituted or unsubstituted C₁₋₄ alkyl, C₁₋₄ alkenyl, PEG ester alkyl, or C₁₋₃ alkyl carboxylate optionally substituted with a protected or free amine;
   R4 is a nucleobase; and
   Ac is an acetyl group.
   Embodiment 24. The compound of embodiment 23, wherein R1 is an alkyl PEG ester.
   Embodiment 25. The compound of embodiment 23, wherein the PEG ester alkyl comprises 10 to 100 ethylene glycol units.
   Embodiment 26. A compound of Formula V:

C—R1-C′ [Formula V]

wherein C and C′ are independently H or

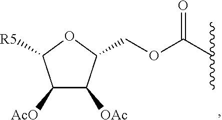

wherein C and C′ are not both H, and:
when one of C or C′ is H, R1 is a substituted or unsubstituted C₈₋₂₄ alkyl, C₈₋₂₄ alkenyl, aryl, alkylaryl, or alkenylaryl; or
when C and C′ are both not H, R1 is a substituted or unsubstituted C₁₋₄ alkyl, C₁₋₄ alkenyl, PEG ester alkyl, or C₁₋₃ alkyl carboxylate optionally substituted with a protected or free amine;
R5 is nicotinamide, dihydronicotinamide, nicotinic acid, nicotinic acid ester, or a reduced form thereof; and
   Ac is an acetyl group.
   Embodiment 27. The compound of embodiment 26, wherein R1 is an alkyl PEG ester.
   Embodiment 28. The compound of embodiment 26, wherein the PEG ester alkyl comprises 10 to 100 ethylene glycol units.
   Embodiment 29. A compound of Formula VI:

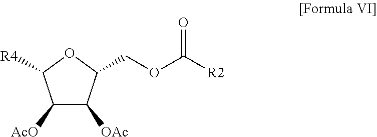

wherein R2 is a substituted or unsubstituted C₈₋₂₄ alkyl, C₈₋₂₄ alkenyl, aryl, alkylaryl, alkenylaryl, or PEG ester alkyl;
R4 is a nucleobase; and
Ac is an acetyl group.
Embodiment 30. The compound of embodiment 29, wherein R2 is a C₁₂ alkyl, a C₁₈ alkenyl, a C₂₀ alkenyl, or a C₂₂ alkenyl.
Embodiment 31. The compound of embodiment 30, wherein

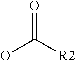

is a substituted or unsubstituted palmitoyl, oleoyl, linoleoyl, linolenoyl, arachidonoyl eicosapentaenoyl, or docosahexaenoyl group.
Embodiment 32. The compound of embodiment 29, wherein

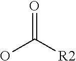

is a succinate ester of retinol.
Embodiment 33. The compound of embodiment 29, wherein

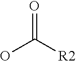

is an omega-3 fatty acid ester.
Embodiment 34. The compound of embodiment 29, wherein R2 is a substituted or unsubstituted phenyl or benzyl.
Embodiment 35. The compound of embodiment 34, wherein R2 is a benzyl that comprises an amino substituent.
Embodiment 36. The compound of embodiment 34, wherein R2 is a para-aminobenzyl.
Embodiment 37. The compound of embodiment 29, wherein R2 is a mycophenolate.
Embodiment 38. The compound of embodiment 29, wherein R2 is an alkyl PEG ester.
Embodiment 39. The compound of embodiment 38, wherein the alkyl PEG ester comprises 10 to 100 ethylene glycol units.
Embodiment 40. A compound of Formula VII:

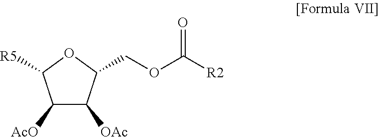

wherein R2 is a substituted or unsubstituted C₈₋₂₄ alkyl, C₈₋₂₄ alkenyl, aryl, alkylaryl, alkenylaryl, or PEG ester alkyl;
R5 is nicotinamide, dihydronicotinamide, nicotinic acid, nicotinic acid ester, or a reduced form thereof; and
Ac is an acetyl group.
Embodiment 41. The compound of embodiment 40, wherein R2 is a C₁₂ alkyl, a C₁₈ alkenyl, a C₂₀ alkenyl, or a C₂₂ alkenyl.
Embodiment 42. The compound of embodiment 41, wherein

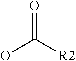

is a substituted or unsubstituted palmitoyl, oleoyl, linoleoyl, linolenoyl, arachidonoyl eicosapentaenoyl, or docosahexaenoyl group.
Embodiment 43. The compound of embodiment 40, wherein

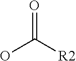

is a succinate ester of retinol.
Embodiment 44. The compound of embodiment 41, wherein

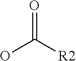

is an omega-3 fatty acid ester.
Embodiment 45. The compound of embodiment 41, wherein R2 is a substituted or unsubstituted phenyl or benzyl.
Embodiment 46. The compound of embodiment 45, wherein R2 is a benzyl that comprises an amino substituent.
Embodiment 47. The compound of embodiment 45, wherein R2 is a para-aminobenzyl.
Embodiment 48. The compound of embodiment 40, wherein R2 is a mycophenolate.
Embodiment 49. The compound of embodiment 40, wherein R2 is an alkyl PEG ester.
Embodiment 50. The compound of embodiment 49, wherein the alkyl PEG ester comprises 10 to 100 ethylene glycol units.
Embodiment 51. A compound of Formula VIII:

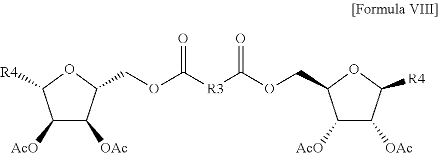

wherein R3 is a substituted or unsubstituted C₁₋₄ alkyl, C₁₋₄ alkenyl, or C₁₋₃ alkyl optionally substituted with a protected or free amine,
or wherein R3 comprises a carbon bonded to (i) a hydroxyl group and (ii) one of

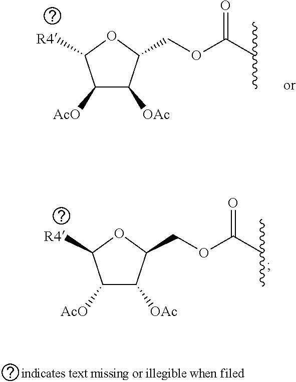

R4, R4′ and R4″ are each independently a nucleobase; and
Ac is an acetyl group.
Embodiment 52. The compound of embodiment 51, wherein R3 is an unsubstituted C₁₋₄ alkyl.
Embodiment 53. The compound of embodiment 51, wherein R3 is C₂ alkenyl.
Embodiment 54. The compound of embodiment 51, wherein R3 is a C₁₋₃ alkyl substituted with a Boc-protected amine.
Embodiment 55. The compound of embodiment 51, wherein R3 is a C₁₋₃ alkyl substituted with an N-Boc protected glutamate or aspartate.
Embodiment 56. The compound of embodiment 51, wherein R3 is a C₃ alkyl substituted with

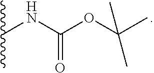 [SMILES: *NC(=O)OC(C)(C)C]

Embodiment 57. The compound of embodiment 51, wherein the compound of Formula VIII is

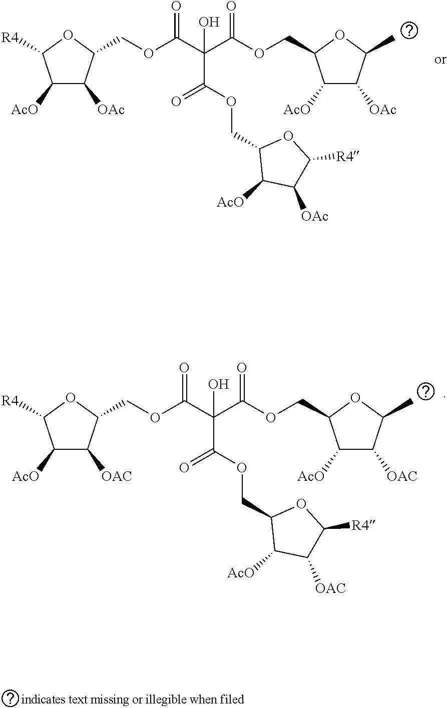

Embodiment 58. A compound of Formula IX:

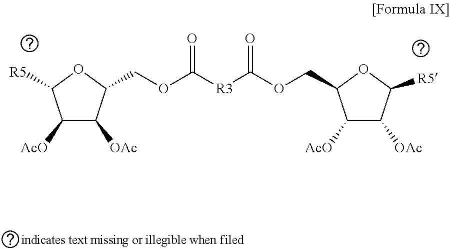

wherein R3 is a substituted or unsubstituted C₁₋₄ alkyl, C₁₋₄ alkenyl, or C₁₋₃ alkyl optionally substituted with a protected or free amine,
or wherein R3 is a carbon bonded to (i) a hydroxyl group and (ii) one of

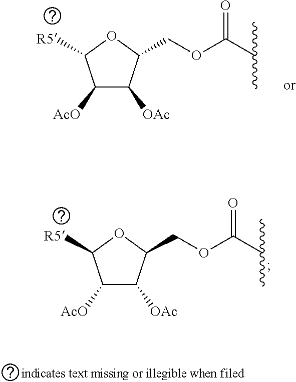

wherein R5, R5′, and R5″ are each independently nicotinamide, dihydronicotinamide, nicotinic acid, nicotinic acid ester, or a reduced form thereof; and
Ac is an acetyl group.
Embodiment 59. The compound of embodiment 58, wherein R3 is an unsubstituted C₁₋₄ alkyl.
Embodiment 60. The compound of embodiment 58, wherein R3 is C₂ alkenyl.
Embodiment 61. The compound of embodiment 58, wherein R3 is a C₁₋₃ alkyl substituted with a Boc-protected amine.
Embodiment 62. The compound of embodiment 58, wherein R3 is a C₁₋₃ alkyl substituted with an N-Boc protected glutamate or aspartate.
Embodiment 63. The compound of embodiment 58, wherein R3 is a C₃ alkyl substituted with

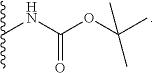 [SMILES: *NC(=O)OC(C)(C)C]

Embodiment 64. The compound of embodiment 58, wherein the compound of Formula IX is:

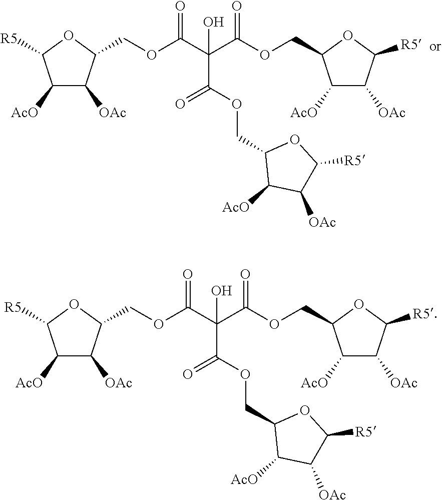

Embodiment 65. A method of making a compound of Formula I, Formula II, or Formula III, comprising:
(a) adding a protecting group to the 5′-carbon of D-ribose, to form a 5′-protected ribose;
(b) acetylating the hydroxy groups at the 1′, 2′, and 3′ carbons of the 5′-protected ribose, to form an acetylated, 5′-protected ribose;
(c) deprotecting the 5′-carbon of the acetylated, 5′-protected ribose, to form an acetylated, 5′-deprotected ribose; and
(d) coupling the acetylated, 5′-deprotected ribose with a reactant comprising R1, R2, and/or R3, to form the compound of Formula I, Formula II, or Formula III.
   Embodiment 66. The method of embodiment 65, wherein the coupling is performed by a mechanochemical reaction.
   Embodiment 67. The method of embodiment 65 or 66, wherein the reactant of (d) comprises an acid chloride, an acid anhydride, a dicarboxylic acid, a monocarboxylic acid, or a combination thereof
   Embodiment 68. A method of making a compound of Formula IV, Formula V, or Formula VI, comprising reacting a compound of Formula I, Formula II, or Formula III with a functionalized nucleobase to form the compound of Formula IV, Formula V, or Formula VI.
   Embodiment 69. A method of making a compound of Formula VII, Formula VIII, or Formula IX, comprising reacting a compound of Formula I, Formula II, or Formula III with a functionalized nicotinamide to form the compound of Formula IV, Formula V, or Formula VI.
   Embodiment 70. The method of embodiment 68 or 69, wherein the reacting is performed by a mechanochemical reaction.
   Embodiment 71. A composition comprising the compound of any one of embodiment 1 to 64 and a pharmaceutically acceptable excipient.
   Embodiment 72. A method of treating nicotinamide adenine dinucleotide (NAD) deficiency in a subject in need thereof, comprising administering the compound of any one of embodiments 26 to 28, any one of embodiments 40 to 50, or any one of embodiments 58 to 64 to the subject.
   Embodiment 73. A method of increasing nicotinamide adenine dinucleotide (NAD) in a subject in need thereof, comprising administering the compound of any one of embodiments 26 to 28, any one of embodiments 40 to 50, or any one of embodiments 58 to 64 to the subject.
   Embodiment 74. The method of embodiment 72 or 73, further comprising administering pterostilbene to the subject.

### EXAMPLES

### Example 1. Synthetic Scheme 1

Exemplary synthesis methods are provided in this example for Synthetic Scheme 1 below.

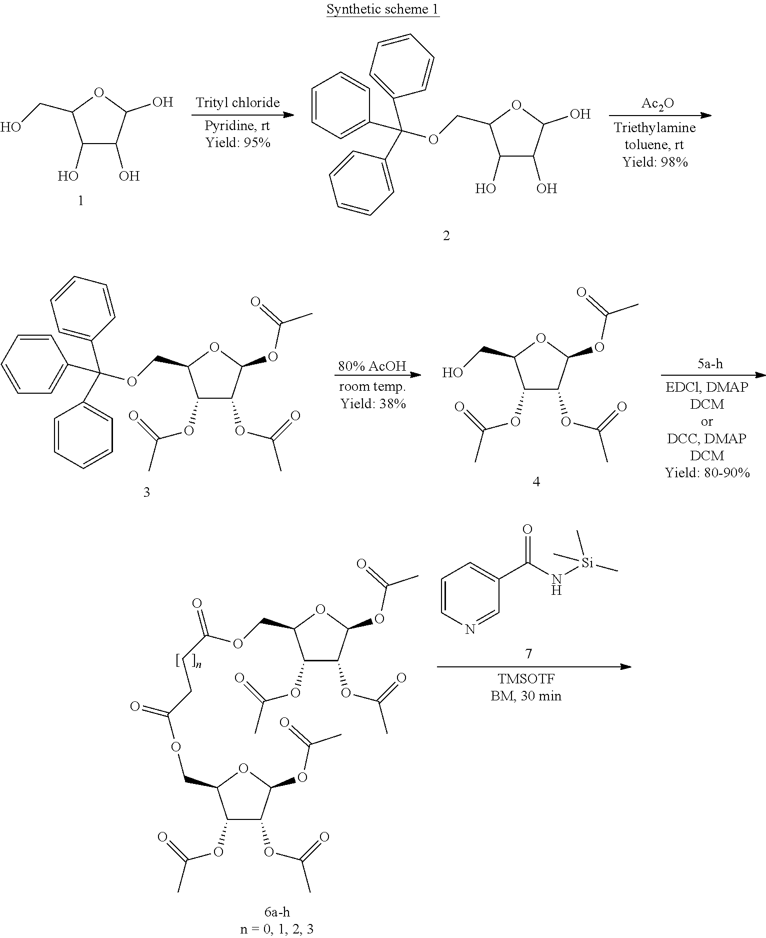

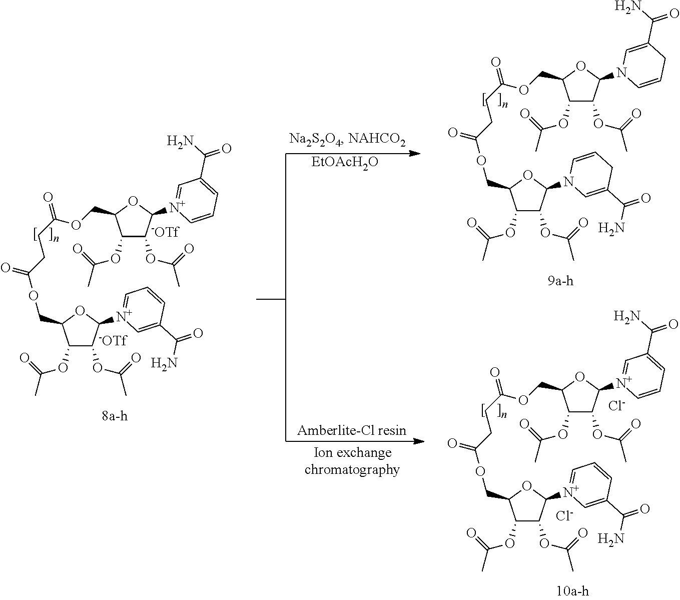

### Synthesis of Compound 2.

A round bottom flask was charged with D-ribose, followed by pyridine under nitrogen atmosphere at room temperature. To this solution, trityl chloride was added and the resulting solution was stirred at room temperature for overnight (16 h). The reaction mixture was then quenched with methanol and stirred for 5-10 min. The resulting mixture was evaporated under reduced pressure to distill off pyridine to afford a pale yellow syrupy residue, which was worked up using DCM and DI water. Organic layer was collected and washed with brine, dried on Na₂SO₄ and evaporated under reduced pressure to obtain the desired compound as a pale yellow syrup. See, e.g., Kristinsson et al., “A novel synthesis of sulfamoyl nucleosides,” Tetrahedron 50(23):6825-6838 (1994).

¹H NMR (400 MHz, CDCl₃), δ, ppm: 3.06 (dd, 1H, 1H′), 3.23 (dd, 1H, 1H′), 3.96 (m, 1H, 4H′), 4.15 (m, 2H, 3H′, 2H′), 5.33 (d, 1H, 1H′), 7.21 (m, 9H), 7.38 (M, 6H). ¹³CNMR (101 MHz, CDCl₃), δ, ppm: 64.0, 71.9, 72.1, 82.8, 87.3, 96.8, 127.8, 128.08, 143.4.

### Synthesis of Compound 3.

A clean and dry round bottom flask was charged with intermediate 2 and toluene. The reaction mixture was then cooled on ice, followed by the addition of acetic anhydride and slow addition of triethylamine. The reaction mixture was then allowed to warm to room temperature and stirred at the same temperature for overnight (16 h). The progress of the reaction mixture was monitored by TLC and after the reaction was complete, reaction mixture was diluted with DI water and toluene and stirred well. Organic layer was collected and washed with brine, dried on Na₂SO₄ and evaporated under reduced pressure to obtain the desired compound as a colorless syrup. See, e.g., Kristinsson et al., Tetrahedron 50(23):6825-6838 (1994).

¹H NMR (400 MHz, CDCl₃), δ, ppm: 1.90 (s, 3H), 1.92 (s, 3H), 2.03 (s, 3H), 3.07-3.11 (dd, 1H, J=4.2, 10.4 Hz), 3.26-3.3 (dd, 1H, J=3.8, 10.3 Hz), 4.24 (m, 1H), 6.12 (brs, 1H), 7.12-7.24 (m, 9H), 7.36-7.38 (m, 6H). ¹³CNMR (101 MHz, CDCl₃), δ, ppm: 20.4, 20.5, 21.0, 63.2, 70.8, 74.3, 86.7, 98.3, 125.2-128.6 (phenyl carbons), 143.6, 169.3, 169.4, 169.6.

### Synthesis of Compound 4.

A clean round bottom flask was charged with intermediate 3, followed by the addition of 80% acetic acid at rt. The resulting mixture was then stirred at room temperature till the disappearance of starting material. After the reaction was complete, acetic acid was distilled off under reduced pressure, followed by the azeotropic distillation to completely remove acetic acid. Then the reaction mixture was diluted with ethyl acetate and subjected to aqueous work-up. Organic layer was collected and washed with brine, dried on Na₂SO₄ and evaporated under reduced pressure to obtain the crude compound as a colorless syrup. The resulting crude compound was purified by column chromatography (Teledyne) using a mixture of hexanes and ethyl acetate. The desired compound was obtained in 40-50% ethyl acetate in hexanes. See, e.g., Winzar et al., “A Simple Synthesis of C-8 Modified 2-Keto-3-deoxy-D-manno-octulosonic Acid (KDO) Derivatives,” Synlett 2010(4):583-586 (2010).

In a specific procedure, a mixture of intermediate 3 was stirred in 80% acetic acid for 3-4 days. A white solid of trityl alcohol was precipitated that was filtered off and the filtrate was evaporated and purified by column chromatography on Teledyne using a mixture of ethyl acetate and hexanes to afford the desired as a colorless syrup.

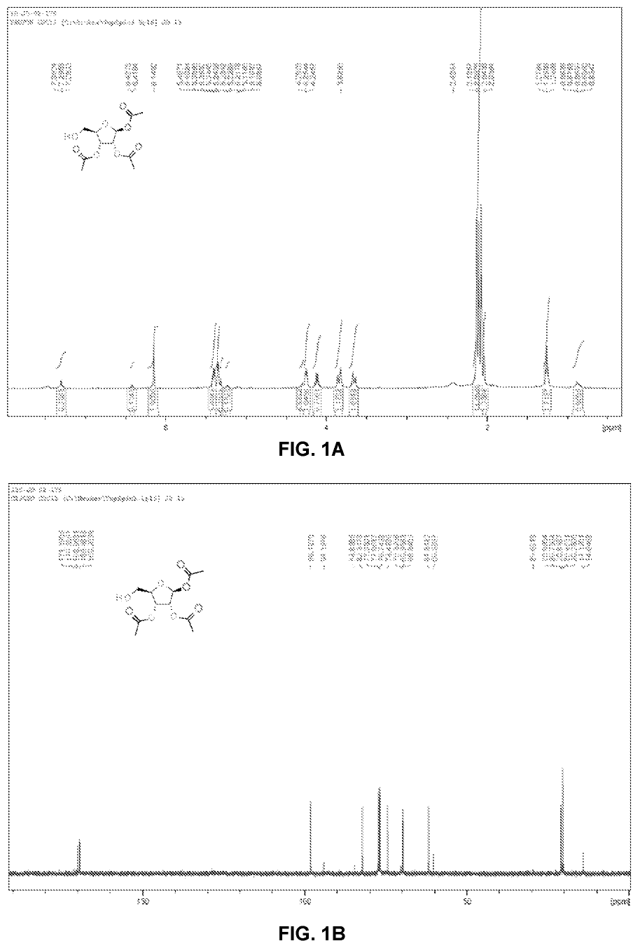

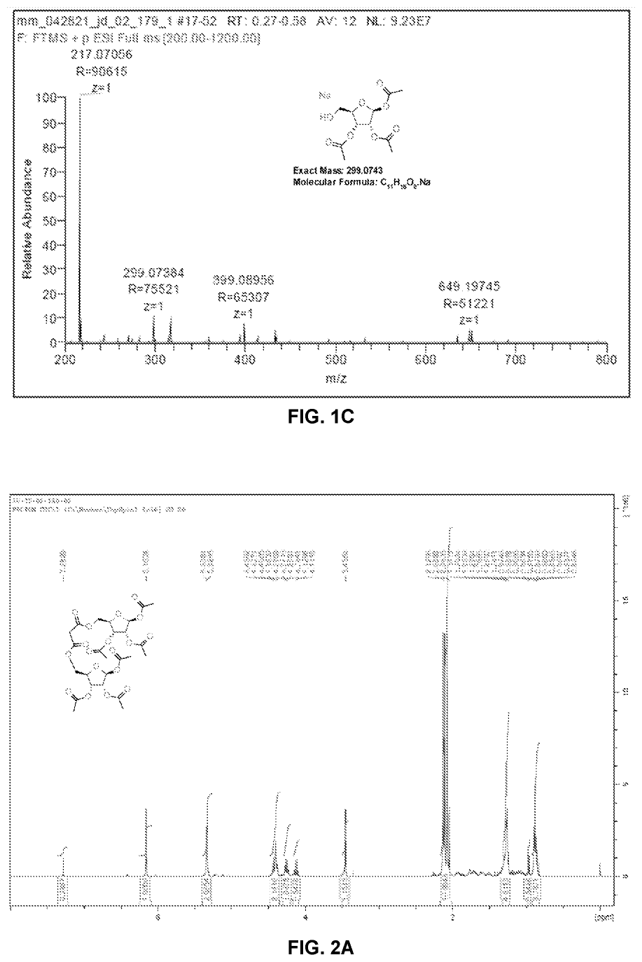

¹H NMR (400 MHz, CDCl₃), δ, ppm: 2.07 (s, 3H), 2.10 (s, 3H), 2.17 (s, 3H), 3.64-3.68 (dd, 1H), 3.84-3.88 (dd, 1H), 4.24-4.27 (m, 1H), 5.34-5.36 (m, 1H), 5.39-5.42 (m, 1H), 6.16 (brs, 1H). ¹³C NMR (100 MHz, CDCl₃) ppm: −20.4, 20.6, 20.9, 61.81, 69.9, 74.4, 82.3, 98.1, 169.4, 169.9, 171.1. HRMS found: 297.0738; Calculated for C₁₁H₁₆O₈ (M+Na): 299.0743. Exemplary spectra shown in FIGS. 1A-1C.

General Synthesis of Compound 6(a-h).

A ball milling jar was charged with intermediate 4 (2 equiv.), followed by compound 5(a-h) (1 equiv.), EDC (2 equiv.), cat. DMAP (0.2 equiv.) and dichloromethane (2-3 drops). The resulting contents were subjected to ball milling conditions on a Retsch MM400 miller for 30 to 60 min at 30 Hz. The reaction mixture was then dissolved into a flask using dichloromethane and then subjected to aqueous work-up. Organic layer was washed with brine, dried on Na₂SO₄ and evaporated under reduced pressure to obtain the crude compound as a colorless syrup. Reaction was monitored by TLC.

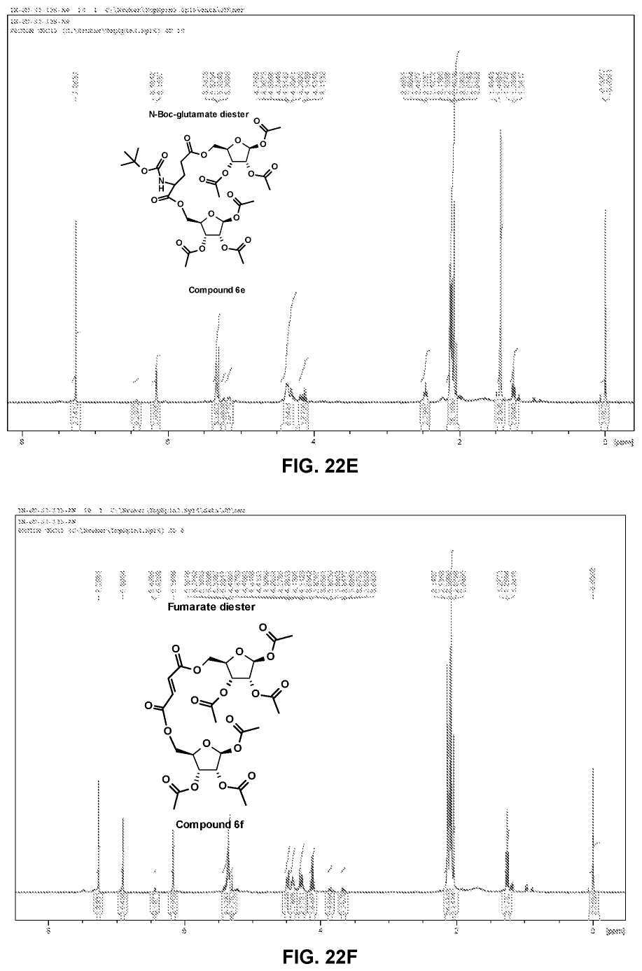

Crude compound obtained was analyzed by ¹H NMR and matched the standard NMRs. Exemplary spectra shown in FIGS. 22A-22G. The resulting compound was deemed pure to proceed to the next step without further purification. Alternatively, the compound was purified by column chromatography (Teledyne) using a mixture of hexanes and ethyl acetate.

Acid anhydrides are also used in lieu of the corresponding acids to obtain the desired products.

### General Synthesis of Compound 7.

A clean dry round bottom flask was charged with nicotinamide, followed by HMDS and the resulting mixture was heated to 110-200° C. for 16 h. Then the reaction mixture was distilled off and used as such for the next step without further purification. See, e.g., Makarov et al., “Scalable syntheses of traceable ribosylated NAD+ precursors,” Org Biomol Chem. 17:8716-8720 (2019).

General Synthesis of Compound 8(a-h).

A ball milling jar was charged with corresponding intermediates 6(a-h) (1 equiv.) and NAM-TMS (compound 7 (2 equiv.)), followed by the addition of trimethylsilyltrifluoromethane sulfonate and 1-2 drops of anhydrous dichloromethane. The resulting mixture was subjected to ball milling conditions on a Retsch MM400 miller for 20-30 min at 30 Hz based on the mono or diester derivative. Reaction progress was monitored by ¹H NMR. After the reaction was completed, reaction mixture was allowed to cool to room temperature and then it was dissolved into a flask using acetone. The resulting solution was distilled off under reduced pressure and triturated with diethyl ether and dried on high vacuum to obtain a yellow foamy and highly hygroscopic mixture.

General Synthesis of Compound 9(a-h).

A clean dry round bottomed flask was charged with corresponding NR esters 8(a-h) under nitrogen followed by a minimum amount of degassed DI water to dissolve the compound. To this solution, solid NaHCO₃ was added, followed by small portion wise addition of Na₂S₂O₄ and the resulting solution was stirred at room temperature for 1-2 min. Then degassed ethyl acetate was added and the resulting mixture was stirred at room temperature for 4 h followed by an aqueous work-up using DI water and ethyl acetate. The aqueous layer was extracted twice with degassed ethyl acetate and organic layers were combined, dried and evaporated under reduced pressure to yield the desired compound.

### General Anion Exchange Protocol for NR Triflate (NR-OTf) to NR Chloride (NR—Cl).

A clear solution of NR triflate in DI water was cooled over ice to 0-5° C. and stirred for 15-20 minutes. To this solution, amberlite resin was added and stirred at 0-5° C. for 2 hours. A glass column was packed with amberlite resin and DI water was added to it and the column was equilibrated with DI water. The stirred solution of NR triflate with the amberlite resin was added to the column and eluted with DI water or a 1:1 mixture of water and acetonitrile. Collected fractions were tested for chloride ions by titration against silver nitrate solution. Desired fractions from the column were combined and evaporated under reduced pressure to yield a white powder of NR-chloride. The resulting product was analyzed by ¹HNMR and ¹⁹FNMR to test the completion of ion exchange from triflate to chloride. The absence of fluorine peak in ¹⁹FNMR confirmed the complete conversion of corresponding triflate salt to chloride. Synthesis of compound 10(a-h) is further described in Example 5.

### Example 2. Synthesis of Compounds 6(a-h)

Exemplary synthesis methods are provided in this example for compounds 6(a-h) of Synthesis Scheme 1 as shown in Example 1. The structures of compounds 6(a-h) are shown below.

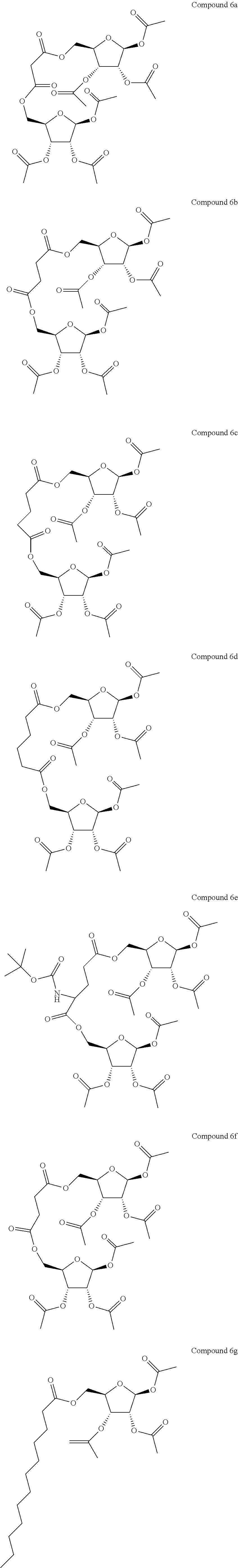

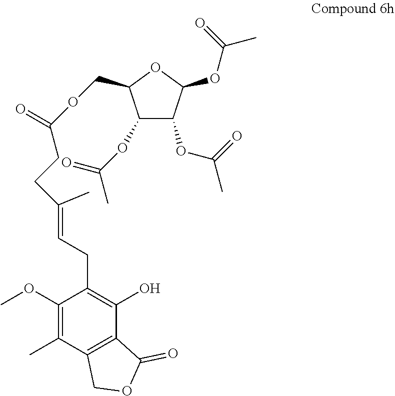 [SMILES: COc1c(C)c2c(c(O)c1C/C=C(\C)CCC(=O)OC[C@H]1O[C@@H](OC(C)=O)[C@H](OC(C)=O)[C@@H]1OC(C)=O)C(=O)OC2]

### Synthesis of Compound 6a (mg Scale).

A stainless steel ball milling jar (1.5 ml) was charged with intermediate 4 (100 mg, 0.37 mmoles) and malonic acid (20 mg, 0.18 mmoles), followed by DCC (77 mg, 0.37 mmoles), DMAP cat. and 1-2 drops of anhydrous dichloromethane. The contents were subjected to ball milling conditions on a Retsch MM400 miller for 80 min at 30 Hz. The jar was allowed to cool down to room temperature and the contents were dissolved into a flask using dichloromethane. White solid that precipitated out was filtered off and the resulting crude compound was then purified by column chromatography on Teledyne using a mixture of ethyl acetate and hexanes to afford the desired intermediate.

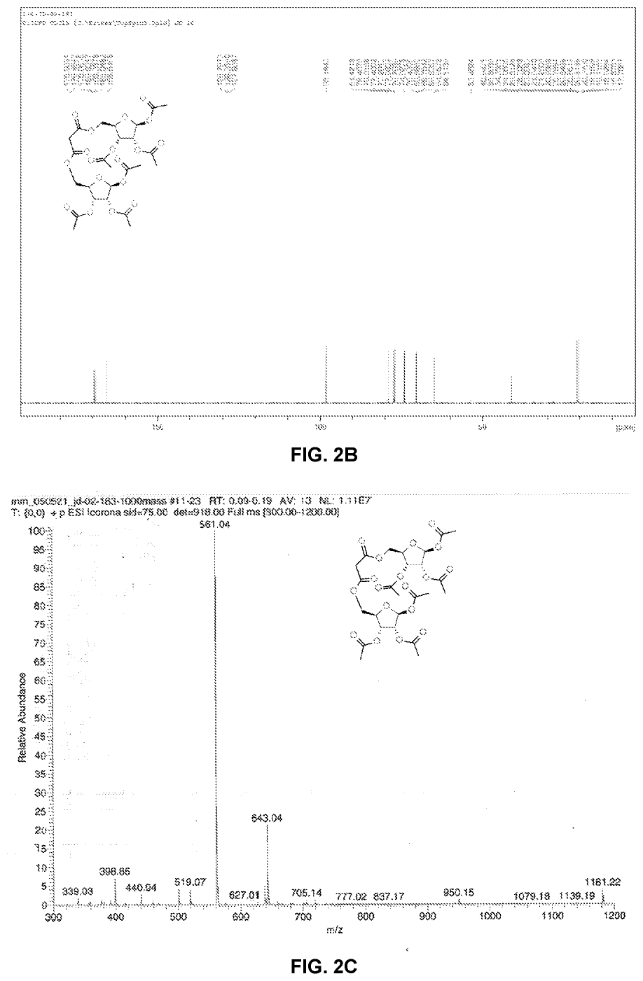

¹H NMR (400 MHz, CDCl₃) ppm: 2.07 (s, 3H), 2.1 (s, 3H), 2.13 (s, 3H), 3.45 (s, 2H), 4.23-4.27 (in, 2H, H5′), 4.37-4.43 (in, 2H, H5′), 4.39 (in, 2H, H4′), 5.32 (in, 2H, H3′), 5.33 (in, 2H, H2′), 6.15 (brd, 2H, H1′). ¹³C NMR (100 MHz, CDCl₃) ppm: 20.3, 20.4, 20.9, 40.9, 64.8, 70.4, 74.0, 79.0, 98.1, 165.5, 168.9, 169.3, 169.6; MS: found m/z=643.04 (M+Na). Calculated for C₂₅H₃₂O₁₈ (M+1): 621.1667; 643.14 (M+Na). Exemplary spectra shown in FIGS. 2A-2C.

### Synthesis of Compound 6a (Gram Scale).

A ball milling jar was charged with intermediate 4 (1 g, 3.76 mmoles, 2 equiv.), followed by malonic acid (200 mg, 1.88 mmoles 1 equiv.), DCC (755 mg, 3.76 mmoles, 2 equiv.), cat. DMAP (45 mg, 0.37 mmoles, 0.2 equiv.) and dichloromethane (300 μL). The resulting contents were subjected to ball milling on a Retsch MM400 miller for 60 min at 30 Hz. Reaction progress was monitored by TLC. Then the reaction mixture was dissolved in to a flask using dichloromethane (DCM). Crude compound in DCM was cooled at 4° C. for 30 min and the DCU impurity was filtered off. The resulting solution was diluted with water and then subjected to aqueous work-up. Organic layer was separated and washed with 10% CuSO₄, followed by water and brine solution. The resulting organic layer was then dried on Na₂SO₄ and evaporated under reduced pressure to obtain the crude compound as a pale yellow syrup (960 mg, yield: 82%). Crude compound was obtained as a mixture of diastereomers comprising of 16% α and 84% β isomers.

¹H NMR (400 MHz, CDCl₃) ppm: 2.07 (s, 3H), 2.1 (s, 3H), 2.13 (s, 3H), 3.45 (s, 2H), 4.23-4.27 (m, 2H, H5′), 4.37-4.43 (m, 2H, H5′), 4.39 (m, 2H, H4′), 5.32 (m, 2H, H3′), 5.33 (m, 2H, H2′), 6.15 (brd, 2H, H1′). ¹³C NMR (100 MHz, CDCl₃) ppm: 20.3, 20.4, 20.9, 40.9, 64.8, 70.4, 74.0, 79.0, 98.1, 165.5, 168.9, 169.3, 169.6; MS: found m/z=643.04 (M+Na). Calculated for C₂₅H₃₂O₁₈ (M+1): 621.1667; 643.14 (M+Na).

### Synthesis of Compound 6b (mg Scale).

A stainless steel ball milling jar (1.5 ml) was charged with intermediate (130 mg, 0.49 mmoles) and succinic acid (25 mg, 0.24 mmoles), followed by EDCI (95.7 mg, 0.49 mmoles), DMAP (6 mg, 0.049 mmoles) and a 2-3 drops of anhydrous dichloromethane. The contents were subjected to ball milling conditions on a Retsch MM400 miller for 30 min at 30 Hz. The jar was allowed to cool down to room temperature and the contents were dissolved into a flask using dichloromethane. The resulting mixture was evaporated under reduced pressure and then purified by column chromatography on Teledyne using a mixture of ethyl acetate and hexanes to afford the desired intermediate.

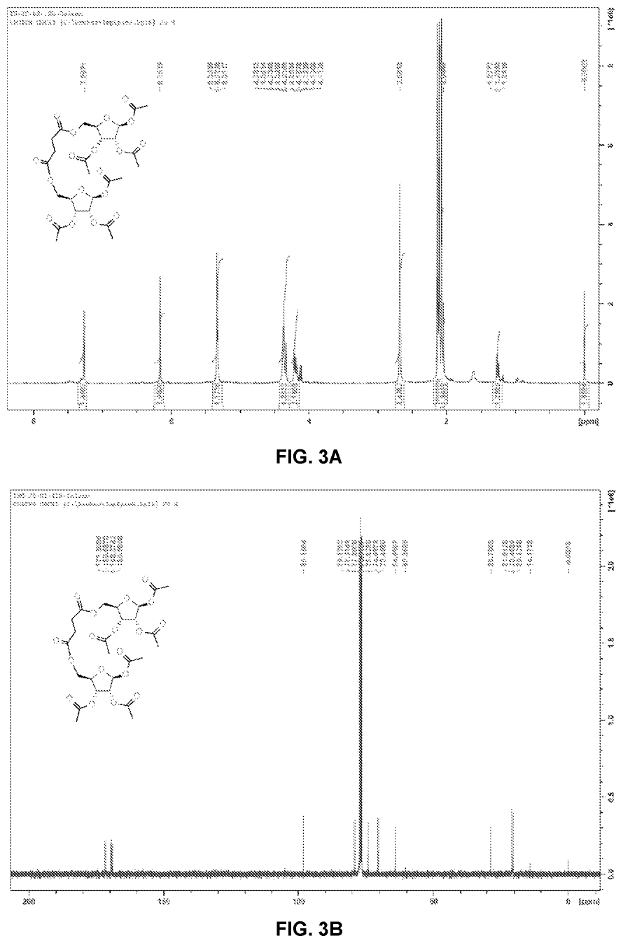

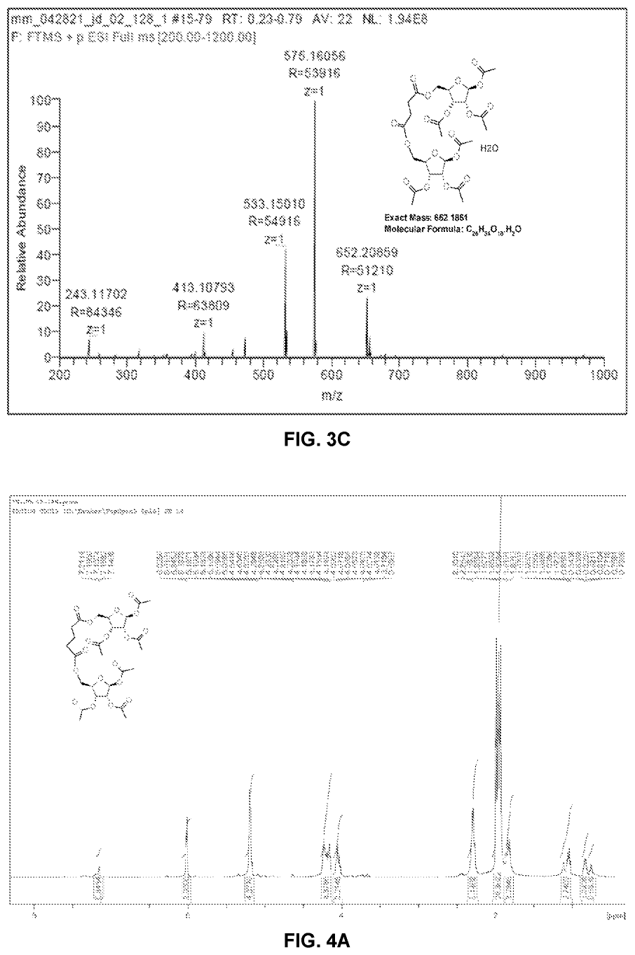

¹H NMR (400 MHz, CDCl₃) ppm: 2.07 (s, 3H), 2.10 (s, 3H), 2.13 (s, 3H), 2.68 (brs, 4H), 4.17-4.21 (dd, 2H, 5H′), 4.32-4.36 (dd, 2H, 5H′), 4.36-4.38 (m, 2H, 4H′), 5.32 (m, 2H, 3′H), 5.33 (m, 2H, 2′H), 6.16 (brd, 2H, 1′H); ¹³C NMR (100 MHz, CDCl₃) ppm: 20.4, 20.46, 21, 28.7, 64, 70.4, 74, 79.1, 98.1, 168.9, 169.3, 169.6, 171.5; MS: found m/z=652.07 (M+H₂O); 657.01 (M+Na). HRMS found: 652.2085. Calculated for C₂₆H₃₄O₁₈ (M+H₂O): 652.1851. Exemplary spectra shown in FIGS. 3A-3C.

### Synthesis of Compound 6b (Gram Scale).

A ball milling jar was charged with intermediate 4 (1.1 g, 3.99 mmoles, 2 equiv.), followed by succinic anhydride (200 mg, 1.99 mmoles, 1 equiv.), EDCI (765 mg, 3.99 mmoles, 2 equiv.), cat. DMAP (49 mg, 0.4 mmoles, 0.2 equiv.) and dichloromethane (300 μL). The resulting contents were subjected to ball milling on a Retsch MM400 miller for 60 min at 30 Hz. Reaction progress was monitored by TLC. Then the reaction mixture was dissolved in to a flask using dichloromethane (DCM) and diluted with water and further subjected to aqueous work-up. Organic layer was separated and washed with 10% CuSO₄, followed by water and brine solution. The resulting organic layer was then dried on Na₂SO₄ and evaporated under reduced pressure to obtain the crude compound as an amber colored syrup (1.2 g, yield: 95%). Crude compound was obtained as a mixture of diastereomers comprising of 11% α and 89% β isomers.

¹H NMR (400 MHz, CDCl₃) ppm: 2.07 (s, 3H), 2.10 (s, 3H), 2.13 (s, 3H), 2.68 (brs, 4H), 4.17-4.21 (dd, 2H, 5H′), 4.32-4.36 (dd, 2H, 5H′), 4.36-4.38 (m, 2H, 4H′), 5.32 (m, 2H, 3′H), 5.33 (m, 2H, 2′H), 6.16 (brd, 2H, 1′H); ¹³C NMR (100 MHz, CDCl₃) ppm: 20.4, 20.46, 21, 28.7, 64, 70.4, 74, 79.1, 98.1, 168.9, 169.3, 169.6, 171.5; MS: found m/z=652.07 (M+H₂O); 657.01 (M+Na). HRMS found: 652.2085. Calculated for C₂₆H₃₄O₁₈ (M+H₂O): 652.1851.

### Synthesis of Compound 6c (mg Scale).

A stainless steel ball milling jar (1.5 ml) was charged with intermediate 4 (100 mg, 0.36 mmoles), and glutaric acid (25 mg, 0.18 mmoles), followed by DCC (74 mg, 0.36 0.36 mmoles), DMAP (cat.) and 1-2 drops of anhydrous dichloromethane. The contents were subjected to ball milling conditions on a Retsch MM400 miller for 80 min at 30 Hz. The jar was allowed to cool down to room temperature and the contents were dissolved into a flask using dichloromethane. White solid that precipitated out was filtered off and the resulting mixture was evaporated under reduced pressure and then purified by column chromatography on Teledyne using a mixture of ethyl acetate and hexanes to afford the desired intermediate.

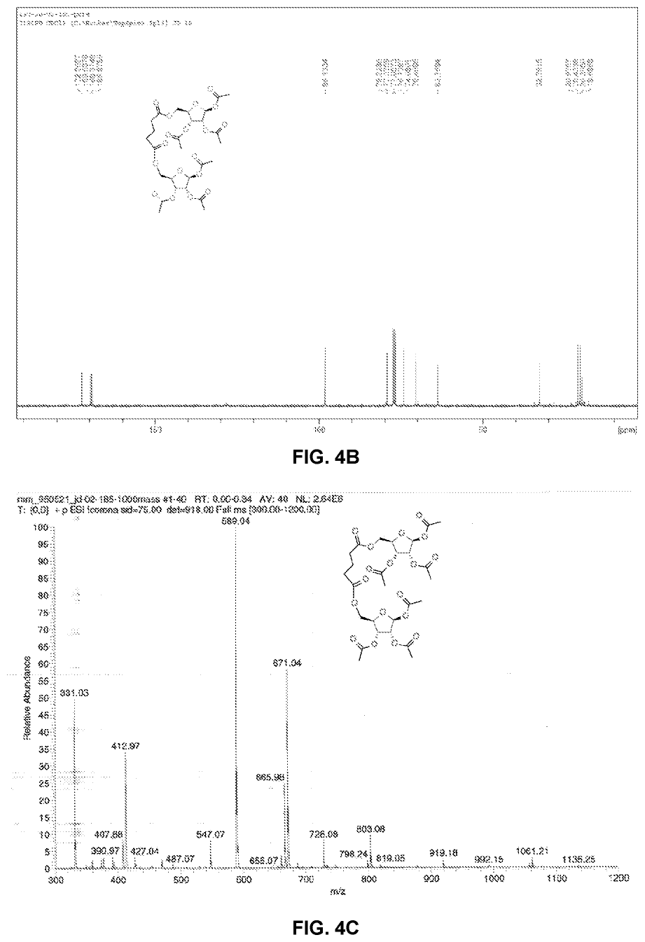

¹H NMR (400 MHz, CDCl₃) ppm: 1.80-1.85 (m, 2H), 1.92 (s, 3H), 1.95 (s, 3H), 1.98 (s, 3H), 2.25-2.3 (m, 4H), 4.01-4.09 (dd, 2H, 5H′), 4.14-4.2 (dd, 2H, 5H′), 4.22 (m, 2H, 4H′), 5.17 (m, 2H, 3H′), 5.19 (m, 2H, 2H′), 6.02 (brd, 2H, 1H′); ¹³C NMR (100 MHz, CDCl₃) ppm: 19.6, 20.3, 20.4, 20.9, 32.7, 63.7, 70.4, 74.0, 79.2, 98.1, 168.9, 169.3, 169.6, 172.2; MS: found m/z=671.04 (M+Na). Calculated for C₂₇H₃₆O₁₈: 650.2058 (M+1); 671.17 (M+Na). Exemplary spectra shown in FIGS. 4A-4C.

### Synthesis of Compound 6c (Gram Scale).

A ball milling jar was charged with intermediate 4 (1 g, 3.6 mmoles, 2 equiv.), followed by glutaric acid (250 mg, 1.8 mmoles, 1 equiv.), DCC (742 mg, 3.6 mmoles, 2 equiv.), cat. DMAP (44 mg, 0.36 mmoles, 0.2 equiv.) and dichloromethane (300 μL). The resulting contents were subjected to ball milling conditions on a Retsch MM400 miller for 60 min at 30 Hz. Reaction progress was monitored by TLC. Then the reaction mixture was dissolved in to a flask using dichloromethane. Crude compound in DCM was cooled at 4° C. for 30 min and the DCU impurity was filtered off. The resulting solution was diluted with water and then subjected to aqueous work-up. Organic layer was separated and washed with 10% CuSO₄, followed by water and brine solution. The resulting organic layer was then dried on Na₂SO₄ and evaporated under reduced pressure to obtain the crude compound as a pale yellow syrup (1 g, yield: 84%). Crude compound was obtained as a mixture of diastereomers comprising of 13% α and 87% β isomers.

¹H NMR (400 MHz, CDCl₃) ppm: 1.80-1.85 (m, 2H), 1.92 (s, 3H), 1.95 (s, 3H), 1.98 (s, 3H), 2.25-2.3 (m, 4H), 4.01-4.09 (dd, 2H, 5H′), 4.14-4.2 (dd, 2H, 5H′), 4.22 (m, 2H, 4H′), 5.17 (m, 2H, 3H′), 5.19 (m, 2H, 2H′), 6.02 (brd, 2H, 1H′); ¹³C NMR (100 MHz, CDCl₃) ppm: 19.6, 20.3, 20.4, 20.9, 32.7, 63.7, 70.4, 74.0, 79.2, 98.1, 168.9, 169.3, 169.6, 172.2; MS: found m/z=671.04 (M+Na). Calculated for C₂₇H₃₆O₁₈: 650.2058 (M+1); 671.17 (M+Na).

### Synthesis of Compound 6d (mg Scale).

A stainless steel ball milling jar (1.5 ml) was charged with intermediate 4 (95 mg, 0.34 mmoles) and adipic acid (25 mg, 0.17 mmoles), followed by EDCI (65 mg, 0.34 mmoles), DMAP (cat.) and 1-2 drops of anhydrous dichloromethane. The contents were subjected to ball milling conditions on a Retsch MM400 miller for 80 min at 30 Hz. The jar was allowed to cool down to room temperature and the contents were dissolved into a flask using dichloromethane and subjected to aqueous work-up using DI water and dichloromethane. Organic layers were collected, washed with brine, dried and evaporated under reduced pressure. The resulting crude compound was then purified by column chromatography on Teledyne using a mixture of ethyl acetate and hexanes to afford the desired intermediate.

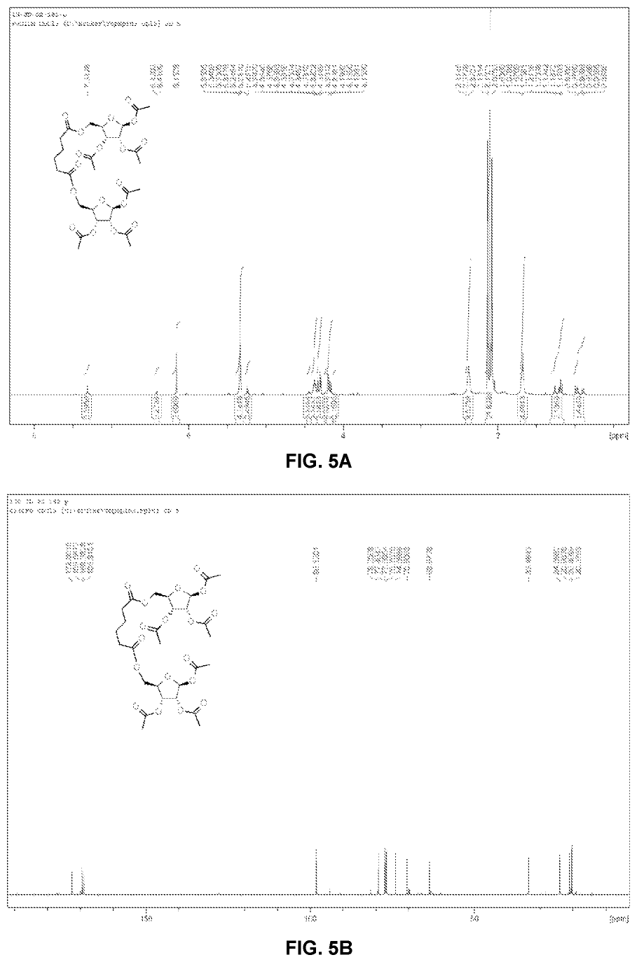

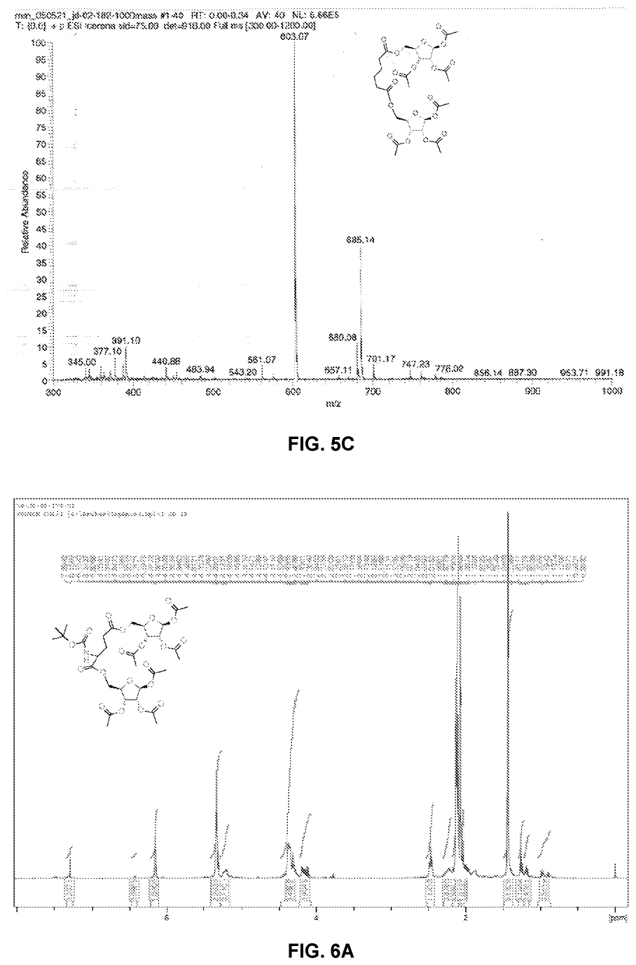

¹H NMR (400 MHz, CDCl₃) ppm: 1.67 (m, 4H), 2.07 (s, 3H), 2.1 (s, 3H), 2.13 (s, 3H), 2.37 (m, 4H), 4.15-4.19 (dd, 2H, 5H′), 4.29-4.33 (dd, 2H, 5H′), 4.34-4.49 (m, 2H, 4H′), 5.31 (m, 2H, 3H′), 5.34 (m, 2H, 2H′), 6.40 (d, 2H); ¹³C NMR (100 MHz, CDCl₃) ppm: 20.3, 20.4, 20.9, 24.0, 33.4, 63.6, 70.5, 74.0, 79.2, 98.1, 168.9, 169.3, 169.5, 172.6; MS: found m/z=685.14 (M+Na). Calculated for C₂₈H₃₈O₁₈: 664.2215 (M+1), 685.19 (M+Na). Exemplary spectra shown in FIGS. 5A-5C.

### Synthesis of Compound 6d (Gram Scale).

A ball milling jar was charged with intermediate 4 (1 g, 3.6 mmoles, 2 equiv.), followed by adipic acid (263 mg, 1.8 mmoles, 1 equiv.), EDCI (694 mg, 3.62 mmoles, 2 equiv.), cat. DMAP (44 mg, 0.36 mmoles, 0.2 equiv.) and dichloromethane (300 μL). The resulting contents were subjected to ball milling on a Retsch MM400 miller for 60 min at 30 Hz. Reaction progress was monitored by TLC. Then the reaction mixture was dissolved in to a flask using dichloromethane (DCM) and diluted with water and further subjected to aqueous work-up. Organic layer was separated and washed with 10% CuSO₄, followed by water and brine solution. The resulting organic layer was then dried on Na₂SO₄ and evaporated under reduced pressure to obtain the crude compound as an amber colored syrup (1.2 g, yield: 90%). Crude compound was obtained as a mixture of diastereomers comprising of 9% α and 91% β isomers.

¹H NMR (400 MHz, CDCl₃) ppm: 1.67 (m, 4H), 2.07 (s, 3H), 2.1 (s, 3H), 2.13 (s, 3H), 2.37 (m, 4H), 4.15-4.19 (dd, 2H, 5H′), 4.29-4.33 (dd, 2H, 5H′), 4.34-4.49 (m, 2H, 4H′), 5.31 (m, 2H, 3H′), 5.34 (m, 2H, 2H′), 6.40 (d, 2H); ¹³C NMR (100 MHz, CDCl₃) ppm: 20.3, 20.4, 20.9, 24.0, 33.4, 63.6, 70.5, 74.0, 79.2, 98.1, 168.9, 169.3, 169.5, 172.6; MS: found m/z=685.14 (M+Na). Calculated for C₂₈H₃₈O₁₈: 664.2215 (M+1), 685.19 (M+Na).

Synthesis of Compound 6e-bis[[(2R,3R,4R,5S)-3,4,5-triacetoxytetrahydrofuran-2-yl]methyl]2-(tert-butoxycarbonylamino)pentanedioate (mg Scale).

A stainless steel ball milling jar (1.5 ml) was charged with intermediate 4 (55 mg, 0.19 mmoles), and N-Boc-glutamic acid (25 mg, 0.1 mmoles), followed by EDCI (38.3 mg, 0.19 mmoles), DMAP (cat.) and 1-2 drops of anhydrous dichloromethane. The contents were subjected to ball milling conditions on a Retsch MM400 miller for 30 min at 30 Hz. The jar was allowed to cool down to room temperature and the contents were dissolved into a flask using dichloromethane. The resulting mixture was evaporated under reduced pressure and then purified by column chromatography on Teledyne using a mixture of ethyl acetate and hexanes to afford the desired intermediate.

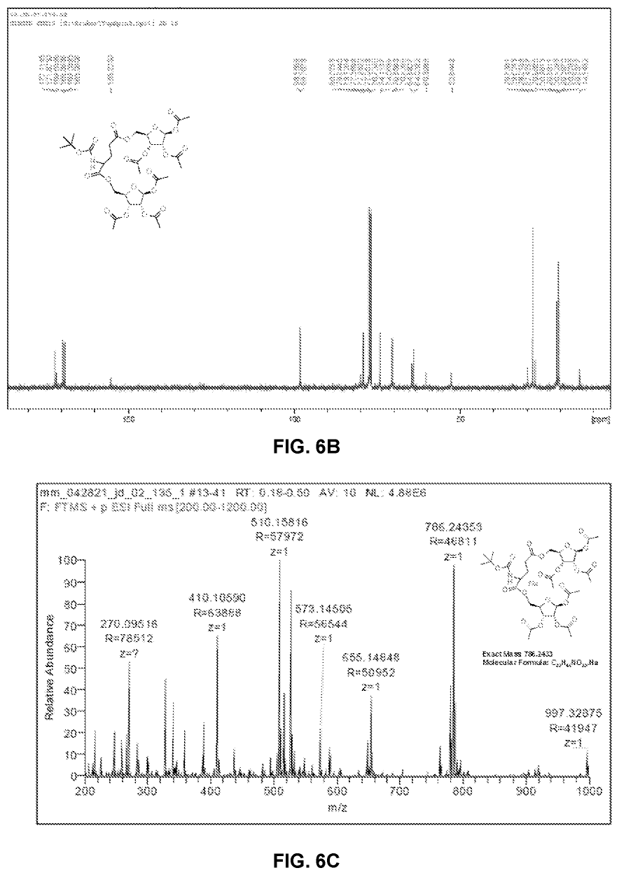

¹H NMR (400 MHz, CDCl₃) ppm: 1.4 (s, 9H), 2.07 (s, 6H), 2.03-2.04 (m, 2H), 2.10-2.13 (brs, 12H), 2.45-2.48 (t, 2H), 4.10-4.18 (m, 2H), 4.28-4.37 (m, 6H), 5.31-5.34 (m, 4H), 6.15 (brd, 2H); ¹³C NMR (100 MHz, CDCl₃) ppm: 20.3, 20.4, 20.9, 27.4, 28.2, 29.7, 64.0, 64.5, 70.4, 70.5, 74.06, 74.1, 79.1, 79.2, 98.0, 98.1, 168.9, 169.3, 169.6, 172.1. MS: found m/z=786.08 (M+Na). HRMS found: 786.2435; Calculated for C₃₂H₄₅NO₂₀: 765.2691 (M+1); 786.2433 (M+Na). Exemplary spectra shown in FIGS. 6A-6C.

### Synthesis of Compound 6e (Gram Scale).

A ball milling jar was charged with intermediate 4 (1 g, 3.62 mmoles, 2 equiv.), followed by N-Boc-glutaric acid (447 mg, 1.81 mmoles, 1 equiv.), EDCI (693 mg, 3.62 mmoles, 2 equiv.), cat. DMAP (45 mg, 0.36 mmoles, 0.2 equiv.) and dichloromethane (300 μL). The resulting contents were subjected to ball milling on a Retsch MM400 miller for 60 min at 30 Hz.

Reaction progress was monitored by TLC. Then the reaction mixture was dissolved in to a flask using dichloromethane (DCM) and diluted with water and further subjected to aqueous work-up. Organic layer was separated and washed with 10% CuSO₄, followed by water and brine solution. The resulting organic layer was then dried on Na₂SO₄ and evaporated under reduced pressure to obtain the crude compound as an amber colored syrup (1.2 g, yield: 88%). Crude compound was obtained as a mixture of diastereomers comprising of 9% α and 91% β isomers.

¹H NMR (400 MHz, CDCl₃) ppm: 1.4 (s, 9H), 2.07 (s, 6H), 2.03-2.04 (m, 2H), 2.10-2.13 (brs, 12H), 2.45-2.48 (t, 2H), 4.10-4.18 (m, 2H), 4.28-4.37 (m, 6H), 5.31-5.34 (m, 4H), 6.15 (brd, 2H); ¹³C NMR (100 MHz, CDCl₃) ppm: 20.3, 20.4, 20.9, 27.4, 28.2, 29.7, 64.0, 64.5, 70.4, 70.5, 74.06, 74.1, 79.1, 79.2, 98.0, 98.1, 168.9, 169.3, 169.6, 172.1. MS: found m/z=786.08 (M+Na). HRMS found: 786.2435; Calculated for C₃₂H₄₅NO₂O: 765.2691 (M+1); 786.2433 (M+Na).

Synthesis of Compound 6f-bis[[(2R,3R,4R,5S)-3,4,5-triacetoxytetrahydrofuran-2-yl]methyl](E)-but-2-enedioate (mg Scale).

A stainless steel ball milling jar (1.5 ml) was charged with intermediate 4 (47 mg, 0.17 mmoles) and fumaric acid (10 mg, 0.08 mmoles), followed by EDCI (33 mg, 0.172 mmoles), DMAP (cat., 0.01 mmoles), and 1-2 drops of anhydrous dichloromethane. The contents were subjected to ball milling conditions on a Retsch MM400 miller for 30 min at 30 Hz. The jar was allowed to cool down to room temperature and the contents were dissolved into a flask using dichloromethane and subjected to aqueous work-up using DI water and dichloromethane. Organic layers were collected, washed with brine, dried and evaporated under reduced pressure. The resulting crude compound was then purified by column chromatography on Teledyne using a mixture of ethyl acetate and hexanes to afford the desired intermediate.

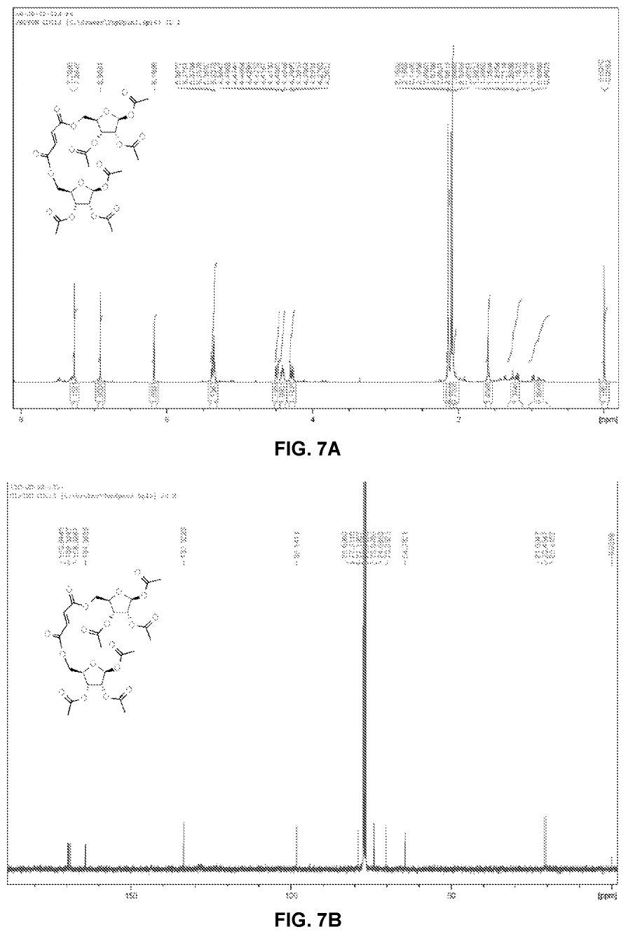

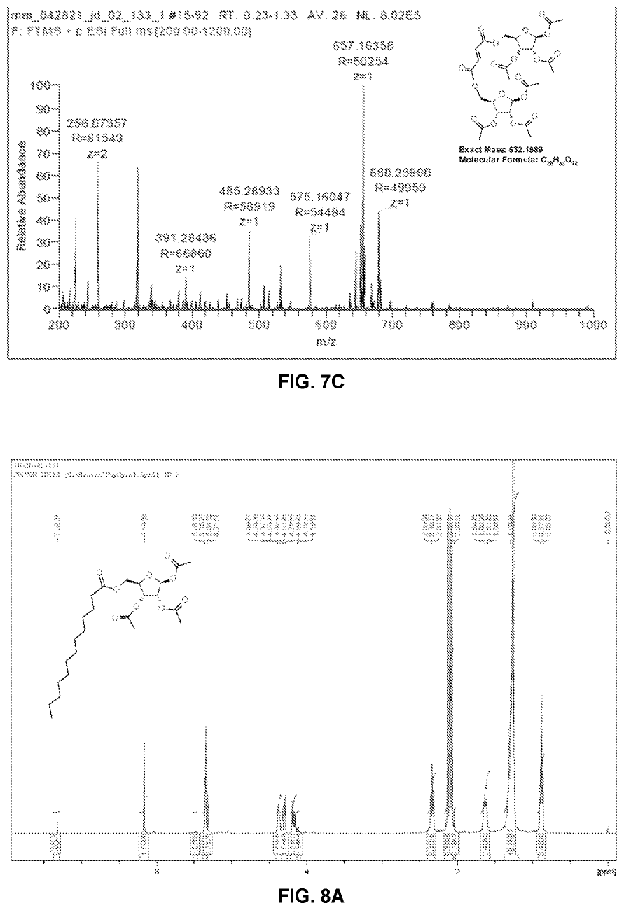

¹H NMR (400 MHz, CDCl₃) ppm: 2.06 (s, 3H), 2.08 (s, 3H), 2.14 (s, 3H), 4.26-4.30 (dd, 2H, 5H′), 4.39-4.40 (m, 2H, 4H′), 4.40-4.41 (dd, 2H, 5H′), 5.35 (m, 2H, 3H′), 5.37 (m, 2H, 2H′), 6.16 (s, 2H, 1H′), 7.28 (d, 2H, olefinic Hs, J=13.5 Hz); ¹³C NMR (100 MHz, CDCl₃) ppm: 20.4, 20.4, 21.0, 64.3, 70.3, 74.0, 78.9, 98.1, 133.5, 164.0, 168.9, 169.3, 169.6; MS: found m/z=678.29 (M+Na). HRMS found: 657.1635 Calculated for C₂₆H₃₂O₁₈: 634.1745 (M+1), 655.1486 (M+Na). Exemplary spectra shown in FIGS. 7A-7C.

### Synthesis of Compound 6f (Gram Scale).

A ball milling jar was charged with intermediate 4 (1 g, 3.62 mmoles, 2 equiv.), followed by fumaric acid (208 mg, 1.81 mmoles, 1 equiv.), EDCI (695 mg, 3.62 mmoles, 2 equiv.), cat. DMAP (44 mg, 0.36 mmoles, 0.2 equiv.) and dichloromethane (300 μL). The resulting contents were subjected to ball milling conditions on a Retsch MM400 miller for 60 min at 30 Hz. Reaction progress was monitored by TLC. Then the reaction mixture was dissolved in to a flask using dichloromethane and then subjected to aqueous work-up. Organic layer was washed with brine, dried on Na₂SO₄ and evaporated under reduced pressure to obtain the crude compound as a colorless syrup, which was eventually purified by column chromatography (teledyne) using a mixture of hexanes and ethyl acetate. Pure compound was obtained as a colorless syrup (550 mg, 48.6% yield). Crude compound was obtained as a mixture of diastereomers comprising of 8% α and 92% β isomers.

¹H NMR (400 MHz, CDCl₃) ppm: 2.06 (s, 3H), 2.08 (s, 3H), 2.14 (s, 3H), 4.26-4.30 (dd, 2H, 5H′), 4.39-4.40 (m, 2H, 4H′), 4.40-4.41 (dd, 2H, 5H′), 5.35 (m, 2H, 3H′), 5.37 (m, 2H, 2H′), 6.16 (s, 2H, 1H′), 7.28 (d, 2H, olefinic Hs, J=13.5 Hz); ¹³C NMR (100 MHz, CDCl₃) ppm: 20.4, 20.4, 21.0, 64.3, 70.3, 74.0, 78.9, 98.1, 133.5, 164.0, 168.9, 169.3, 169.6; MS: found m/z=678.29 (M+Na). HRMS found: 657.1635 Calculated for C₂₆H₃₂O₁₈: 634.1745 (M+1), 655.1486 (M+Na).

Synthesis of Compound 6g—[(2R,3R,4R,5S)-3,4,5-triacetoxytetrahydrofuran-2-yl]methyltridecanoate (Protocol 1).

A stainless steel ball milling jar (1.5 ml) was charged with intermediate 4 (200 mg, 0.72 mmoles) and lauric acid (145 mg, 0.72 mmoles) followed by EDCI (207 mg, 1.08 mmoles), DMAP cat. and anhydrous dichloromethane. The contents were subjected to ball milling conditions on a Retsch MM400 miller for 30 min at 30 Hz. The jar was allowed to cool down to room temperature and the contents were dissolved into a flask using dichloromethane. The resulting mixture was evaporated under reduced pressure and then purified by column chromatography on Teledyne using a mixture of ethyl acetate and hexanes to afford the desired intermediate.

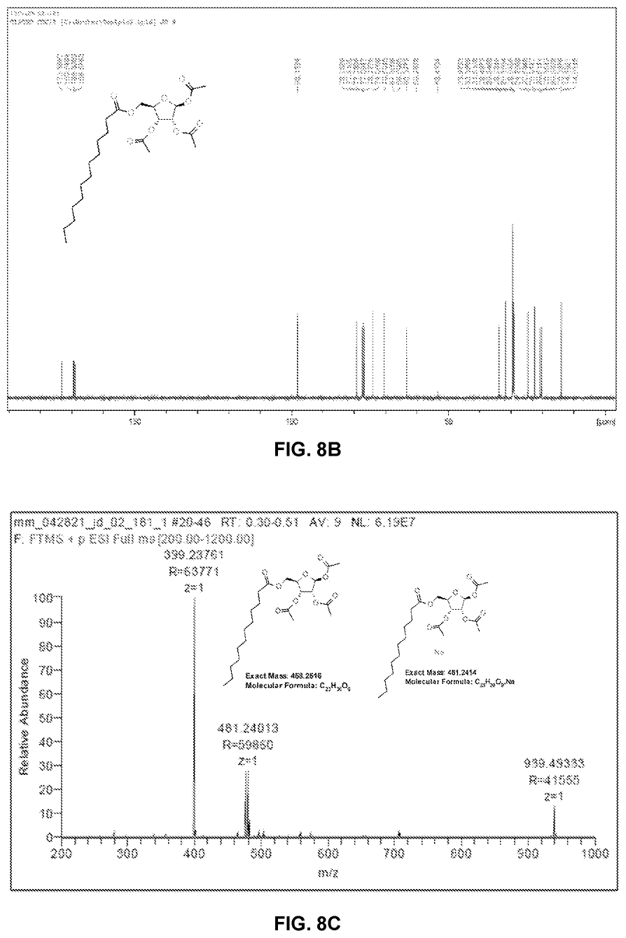

¹H NMR (400 MHz, CDCl₃) ppm: 0.87 (t, 3H, J=6.7 HZ), 1.28 (m, 16H), 1.62 (m, 2H), 2.07 (s, 3H), 2.09 (s, 3H), 2.12 (s, 3H), 2.33 (t, 2H, 7.5 Hz), 4.1-4.32 (dd, 2H, 5H′), 4.38 (m, 1H, 4H′), 5.32 (m, 1H, 3H′), 5.34 (m, 1H, 2H′), 6.16 (d, 1H, 1H′); ¹³C NMR (100 MHz, CDCl₃) ppm: 14.1, 20.3, 20.3, 20.9, 22.5, 24.7, 29.0, 29.1, 29.2, 29.3, 29.4, 31.8, 33.9, 63.3, 70.5, 74.0, 79.2, 98.1, 168.8, 169.3, 169.5, 173.1; MS: found m/z=481.19 (M+Na). HRMS found: 481.2401; Calculated for C₂₄H₄₀O₉: 460.2672 (M+1); 481.2414 (M+Na). Exemplary spectra shown in FIGS. 8A-8C.

### Synthesis of Compound 6g (Protocol 2).

A ball milling jar was charged with intermediate 4 (1 equiv.), followed by lauric acid (1 equiv.), EDCI (1 equiv.), cat. DMAP (0.2 equiv.) and dichloromethane (2-3 drops). The resulting contents were subjected to ball milling conditions on a Retsch MM400 miller for 45 min at 30 Hz. Reaction progress was monitored by TLC Then the reaction mixture was dissolved in to a flask using dichloromethane and then subjected to aqueous work-up. Organic layer was washed with brine, dried on Na₂SO₄ and evaporated under reduced pressure to obtain the crude compound as a colorless syrup, which was eventually purified by column chromatography (teledyne) using a mixture of hexanes and ethyl acetate. Exemplary spectra shown in FIG. 22G.

¹H NMR (400 MHz, CDCl₃) ppm: 0.87 (t, 3H, J=6.7 HZ), 1.28 (m, 16H), 1.62 (m, 2H), 2.07 (s, 3H), 2.09 (s, 3H), 2.12 (s, 3H), 2.33 (t, 2H, 7.5 Hz), 4.1-4.32 (dd, 2H, 5H′), 4.38 (m, 1H, 4H′), 5.32 (m, 1H, 3H′), 5.34 (m, 1H, 2H′), 6.16 (d, 1H, 1H′); ¹³C NMR (100 MHz, CDCl₃) ppm: 14.1, 20.3, 20.3, 20.9, 22.5, 24.7, 29.0, 29.1, 29.2, 29.3, 29.4, 31.8, 33.9, 63.3, 70.5, 74.0, 79.2, 98.1, 168.8, 169.3, 169.5, 173.1; MS: found m/z=481.19 (M+Na). HRMS found: 481.2401; Calculated for C₂₄H₄₀O₉: 460.2672 (M+1); 481.2414 (M+Na).

### Synthesis of Compound 6h.

A clean dry ball milling jar was charged with intermediate 4 (200 mg, 0.72 mmoles), and mycophenolic acid (232 mg, 0.72 mmoles), followed by DCC (148 mg, 0.72 mmoles), DMAP (cat.) and 1-2 drops of anhydrous dichloromethane. The contents were subjected to ball milling conditions on a Retsch MM400 miller for 60 min at 30 Hz. The jar was allowed to cool down to room temperature and the contents were dissolved into a flask using dichloromethane. White solid that precipitated out was filtered off and the resulting mixture was evaporated under reduced pressure and then purified by column chromatography on Teledyne using a mixture of ethyl acetate and hexanes to afford the desired compound.

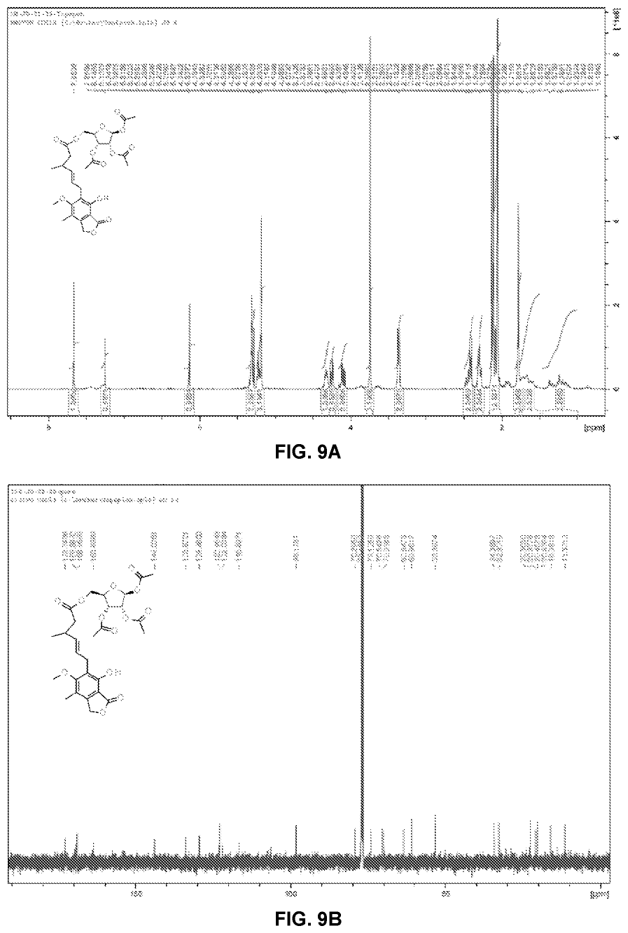

¹H NMR (400 MHz, CDCl₃) ppm: 2.05 (s, 3H), 2.05 (s, 3H), 2.10 (s, 3H), 2.13 (s, 3H), 2.27-2.31 (br m, 2H), 2.39-2.47 (br m, 2H), 3.36 (d, 2h, J=6.8 Hz), 3.74 (s, 3H), 4.07-4.11 (dd, 1H, 5H′), 4.23-4.27 (dd, 1H, 5H′), 4.3-4.34 (m, 1H, 4H′), 5.22 (m, 1H, 3H′), 5.22 (s, 2H, lactone 2H), 5.31 (m, 1H, 3H′), 5.32 (m, 1H, olefinic H), 6.13 (s, 1H, 1H′); ¹³C NMR (100 MHz, CDCl₃) ppm: 11.5, 16.0, 20.43, 20.45, 20.9, 22.5, 32.8, 34.3, 53.3, 60.9, 63.5, 70.0, 70.5, 74.1, 79.2, 98.1, 116.6, 122.0, 122.9, 129.4, 133.8, 144.0, 163.6, 168.9, 169.3, 172.7. Exemplary spectra shown in FIGS. 9A-9B.

### Example 3. Synthesis of Compounds 8(a-h)

Exemplary synthesis methods are provided in this example for compounds 8(a-h) of Synthesis Scheme 1 as shown in Example 1. The structures of compounds 8(a-h) are shown below.

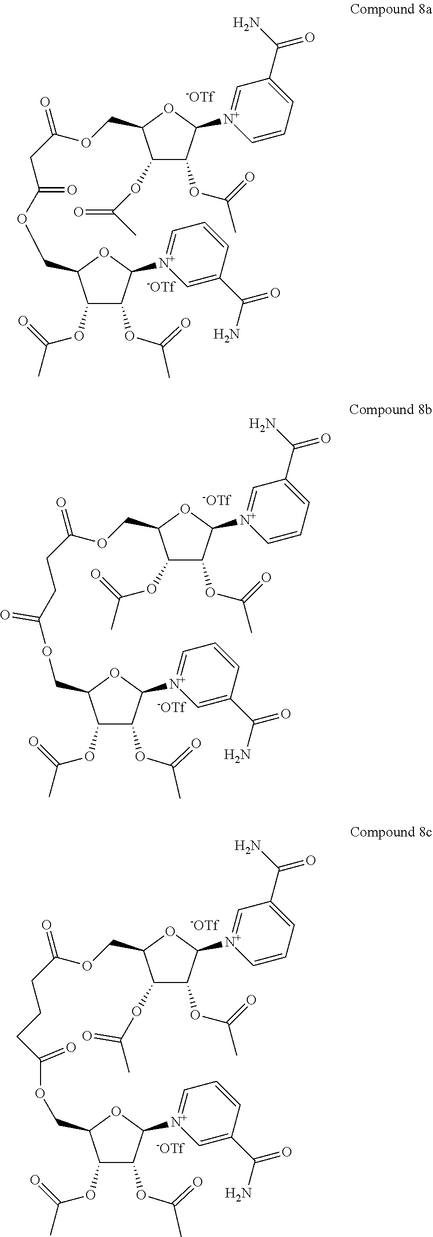

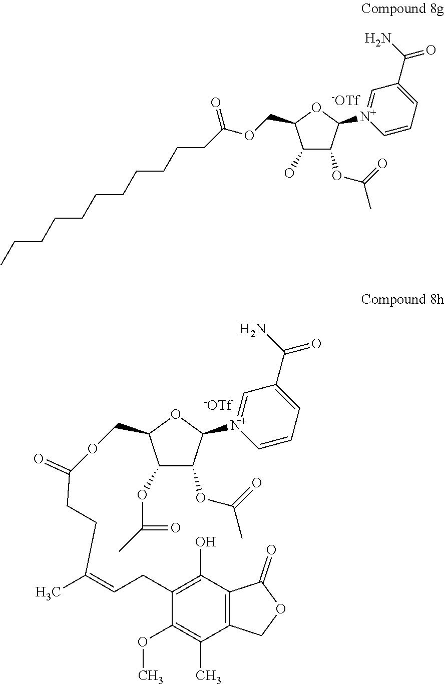 [SMILES: CCCCCCCCCCCC(=O)OC[C@H]1O[C@@H]([n+]2cccc(C(N)=O)c2)[C@H](OC(C)=O)[C@@H]1O ; COc1c(C)c2c(c(O)c1C/C=C(/C)CCC(=O)OC[C@H]1O[C@@H]([n+]3cccc(C(N)=O)c3)[C@H](OC(C)=O)[C@@H]1OC(C)=O)C(=O)OC2 ; "-OTf" ; "-OTf"]

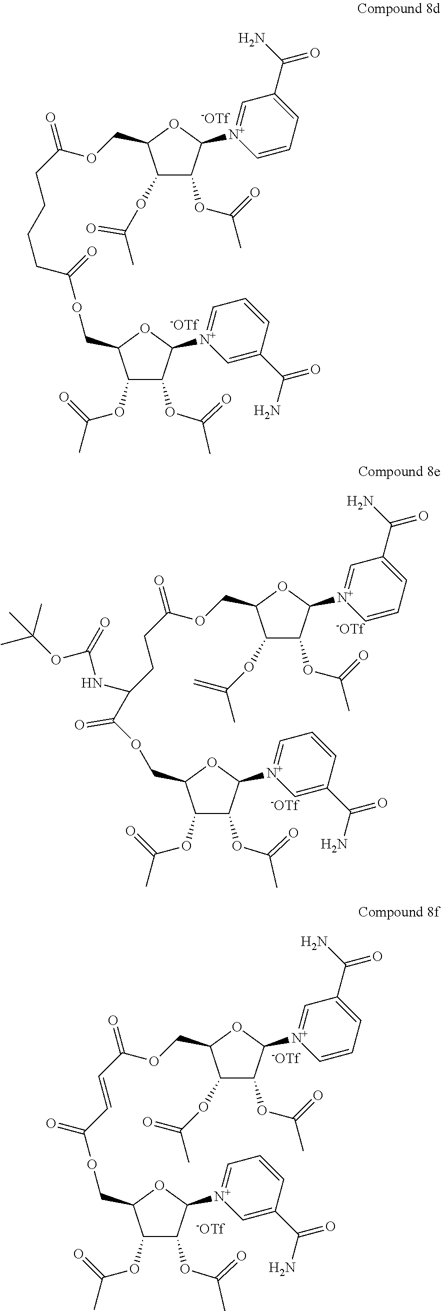

### Synthesis of Compound 8a.

A ball milling jar was charged with intermediate 8a (70 mg, 0.112 mmoles) and compound 7 (43 mg, 0.22 mmoles), followed by the addition of trimethylsilyltrifluoromethane sulfonate (81 μL, 0.44 mmoles) and 1-2 drops of anhydrous dichloromethane. The resulting mixture was subjected to ball milling conditions on a Retsch MM400 miller for 30 min at 30 Hz. After 30 min, reaction mixture was allowed to cool to room temperature and then it was dissolved into a flask using acetone. The resulting solution was distilled off under reduced pressure, triturated with diethyl ether and dried to obtain compound 8a a yellow foamy and highly hygroscopic solid.

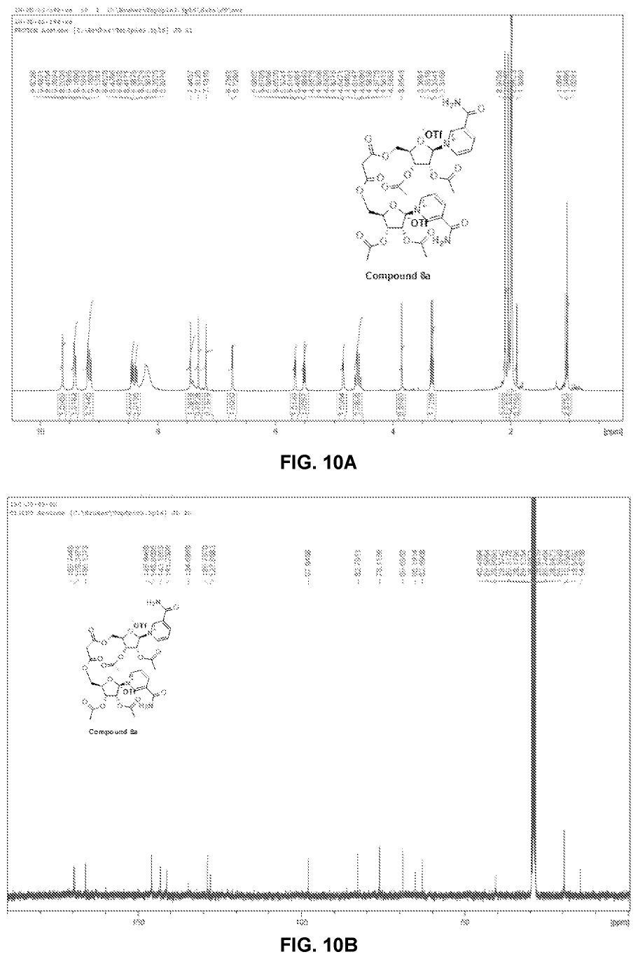

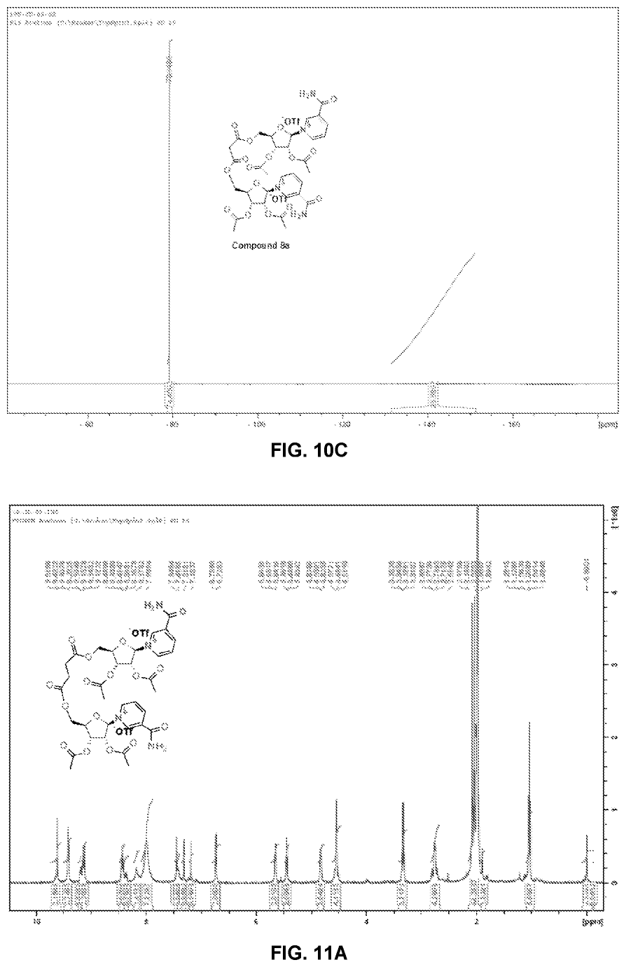

¹H NMR (400 MHz, Acetone-d6) ppm: 2.01 (s, 6H), 2.02 (s, 3H), 2.07 (s, 3H), 3.85 (s, 2H), 4.54-4.64 (dd, 4H, J=2.76 Hz, J=2.44 Hz), 4.83-4.86 (m, 2H), 5.49-5.52 (t, 2H, J=5.6 Hz), 5.65-5.68 (m, 2H), 6.72 (d, 2H, J=3.68 Hz), 8.41-8.45 (m, 2H), 9.13-9.2 (m, 2H), 9.39-9.42 (m, 2H), 9.62 (s, 2H); ¹³C NMR (100 MHz, Acetone-d6) ppm: 19.51, 19.54, 40.48, 62.99, 69.05, 76.11, 82.79, 97.94, 127.86, 128.75, 141.25, 143.18, 145.94, 166.13, 169.34, 169.74. ¹⁹F (376 MHz, Acetone-d6) ppm: −79.14. Exemplary spectra are shown in FIGS. 10A-10C.

### Synthesis of Compound 8b.

A ball milling jar was charged with intermediate 8b (100 mg, 0.157 mmoles) and compound 7 (61 mg, 0.315 mmoles), followed by the addition of trimethylsilyltrifluoromethane sulfonate (114 μL, 0.394 mmoles) and 1-2 drops of anhydrous dichloromethane. The resulting mixture was subjected to ball milling conditions on a Retsch MM400 miller for 30 min at 30 Hz. After 30 min, reaction mixture was allowed to cool to room temperature and then it was dissolved into a flask using acetone. The resulting solution was distilled off under reduced pressure, triturated with diethyl ether and dried to obtain compound 8b a yellow foamy and highly hygroscopic solid.

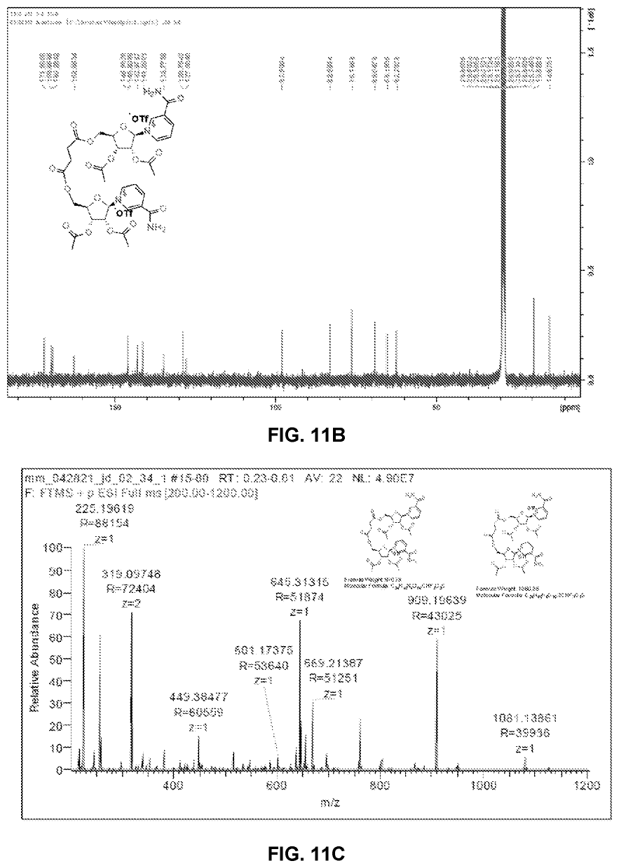

¹H NMR (400 MHz, Acetone-d6) ppm: 9.61 (s, 2H), 9.41 (m, 2H), 9.14 (m, 2H), 8.43 (t, 2H, J=14 Hz), 7.99 (brs, −2NH Hs), 6.72 (d, 2H, J=3.4 Hz), 5.65 (t, 2H, J=8.8 Hz), 5.44 (t, 2H, J=5.5 Hz), 4.82-4.83 (m, 2H), 4.51-4.57 (m, 4H), 2.71-2.8 (m, 4H), 1.98-2.07 (s, 12H); ¹³C NMR (100 MHz, Acetone-d6) ppm: 171.9, 169.6, 169.2, 162.6, 145.9, 142.9, 141.2, 134.7, 128.7, 127.8, 97.9, 82.9, 76.1, 69.0, 65.1, 62.2, 19.5, 14.6. MS: found m/z=909.08 (M+OTf⁻). HRMS found: 909.1963; Calculated for C₂₈H₃₄O₁₃: 910.2038 (M+OTf⁻). Exemplary spectra are shown in FIGS. 11A-11C.

### Synthesis of Compound 8c.

A ball milling jar was charged with intermediate 8c (30 mg, 0.04 mmoles) and compound 7 (18 mg, 0.09 mmoles), followed by the addition of trimethylsilyltrifluoromethane sulfonate (35 μL, 0.185 mmoles) and 1-2 drops of anhydrous dichloromethane. The resulting mixture was subjected to ball milling conditions on a Retsch MM400 miller for 30 min at 30 Hz. After 30 min, reaction mixture was allowed to cool to room temperature and then it was dissolved into a flask using acetone. The resulting solution was distilled off under reduced pressure, triturated with diethyl ether and dried to obtain compound 8c a yellow foamy and highly hygroscopic solid.

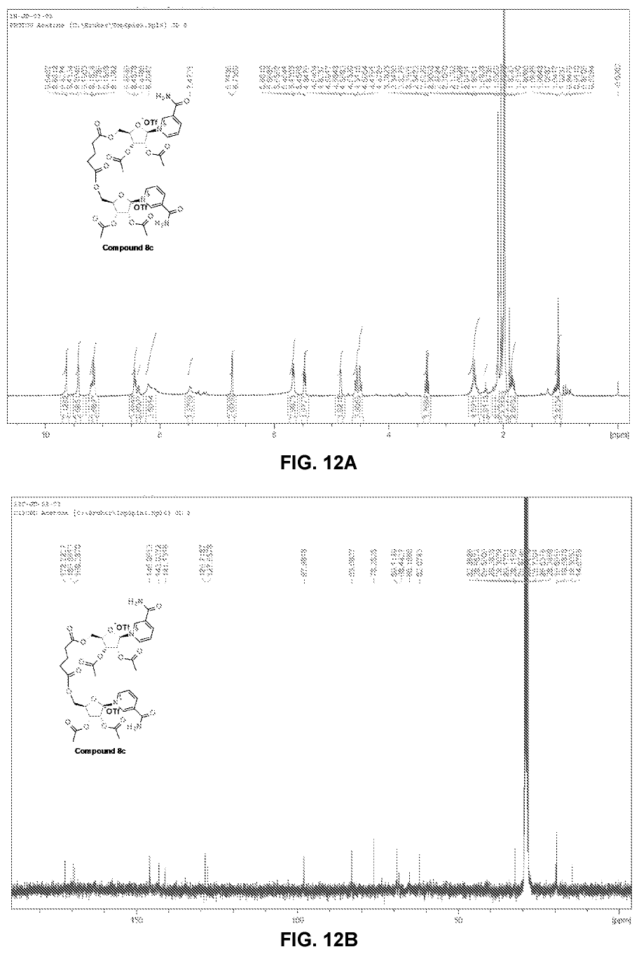

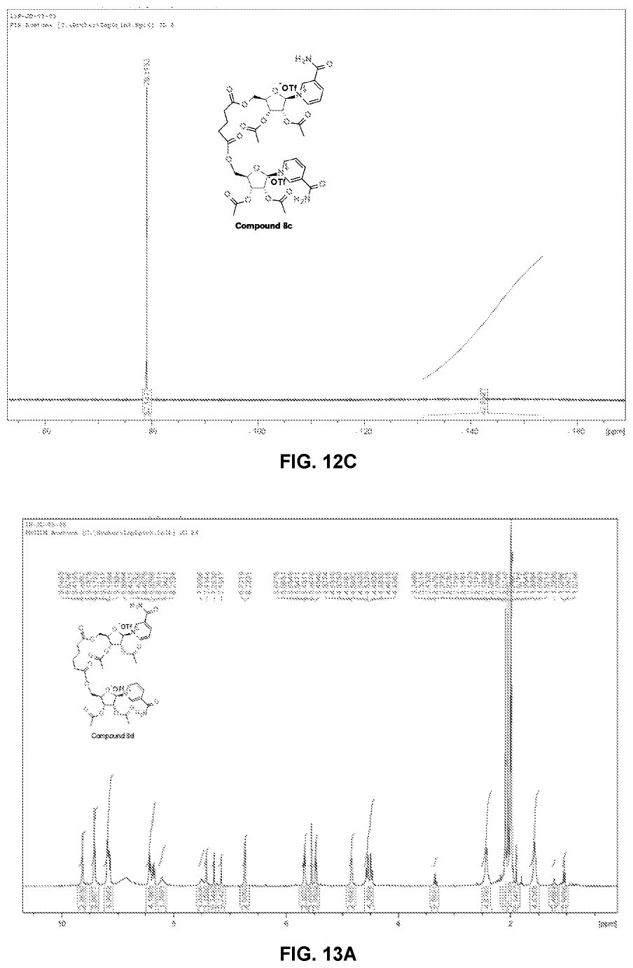

¹H NMR (400 MHz Acetone-d6) ppm: 1.80-1.87 (m, 2H), 2.04 (s, 6H), 2.09 (s, 6H), 2.48-2.54 (m, 4H), 4.47-4.49 (dd, 4H, J=2.96, J=2.48 Hz,) 4.83-4.83 (m, 2H), 5.45-5.48 (t, 2H, J=5.56 Hz), 5.65-5.68 (m, 2H), 6.72 (d, 2H, J=3.44 Hz), 8.41-8.45 (m, 2H), 9.13-9.17 (m, 2H), 9.41-9.42 (m, 2H), 9.63 (s, 2H). ¹³C NMR (100 MHz, Acetone-d6) ppm: 19.5, 19.53, 19.69, 32.38, 62.07, 69.11, 76.25, 83.08, 97.98, 127.85, 128.71, 141.13, 143.03, 145.95, 169.23, 169.68, 172.22. ¹⁹F (376 MHz, Acetone-d6) ppm: −79.11. Exemplary spectra are shown in FIGS. 12A-12C.

### Synthesis of Compound 8d.

A ball milling jar was charged with intermediate 8d (40 mg, 0.06 mmoles) and compound 7 (23.4 mg, 0.12 mmoles), followed by the addition of trimethylsilyltrifluoromethane sulfonate (44 μL, 0.24 mmoles). The resulting mixture was subjected to ball milling conditions on a Retsch MM400 miller for 30 min at 30 Hz. After 30 min, reaction mixture was allowed to cool to room temperature and then it was dissolved into a flask using acetone. The resulting solution was distilled off under reduced pressure, triturated with diethyl ether and dried to obtain compound 8d a highly hygroscopic solid.

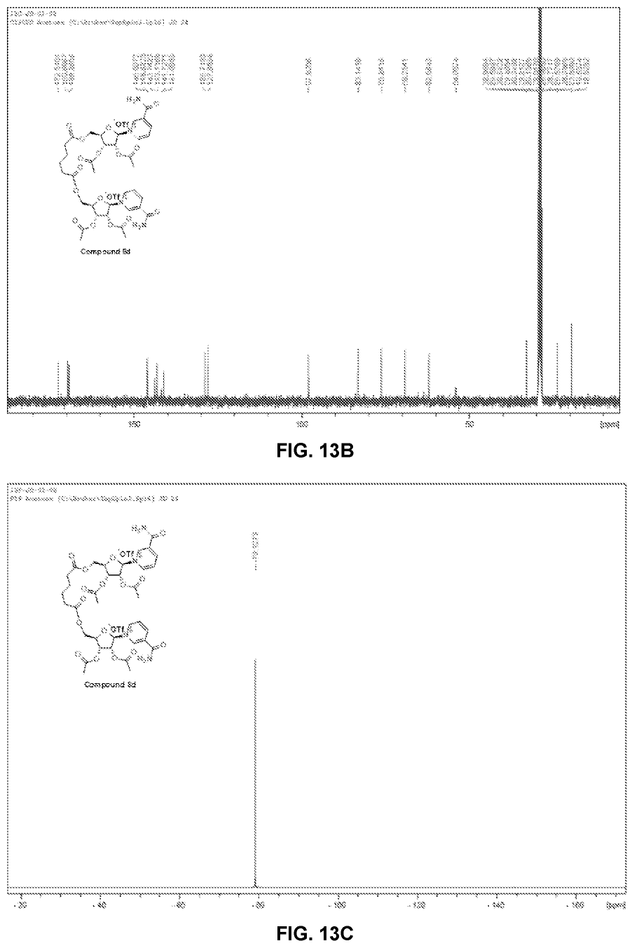

¹H NMR (400 MHz Acetone-d6) ppm: 1.56-1.61 (m, 4H), 2.05 (s, 6H), 2.10 (s, 6H), 2.41-2.46 (m, 4H), 4.46-4.58 (dd, 4H, J=2.76 Hz, 2.16 Hz), 4.83-4.85 (m, 2H), 5.46-5.49 (t, 2H, J=5.42 Hz), 5.67-5.69 (m, 2H), 6.72 (d, 2H, J=3.52 Hz), 8.42-8.45 (m, 2H), 9.14-9.21 (m, 2H), 9.41-9.43 (m, 2H), 9.64 (s, 2H); ¹³C NMR (100 MHz, Acetone-d6) ppm: 19.52, 19.55, 23.83, 32.96, 62.08, 69.21, 76.24, 83.14, 97.92, 127.88, 128.71, 141.06, 143.11, 143.74, 145.82, 146.0, 169.28, 169.69, 172.51. ¹⁹F (376 MHz, Acetone-d6) ppm: −79.12. Exemplary spectra are shown in FIGS. 13A-13C.

### Synthesis of Compound 8e.

A ball milling jar was charged with intermediate 6e (200 mg, 0.262 mmoles) and compound 7 (100 mg, 0.52 mmoles), followed by the addition of trimethylsilyltrifluoromethane sulfonate (232 μL, 1.31 mmoles). The resulting mixture was subjected to ball milling conditions on a Retsch MM400 miller for 40 min at 30 Hz. After 40 min, reaction mixture was allowed to cool to room temperature and then it was dissolved into a flask using acetone. The resulting solution was distilled off under reduced pressure, triturated with diethyl ether and dried to obtain compound 8e a highly hygroscopic solid.

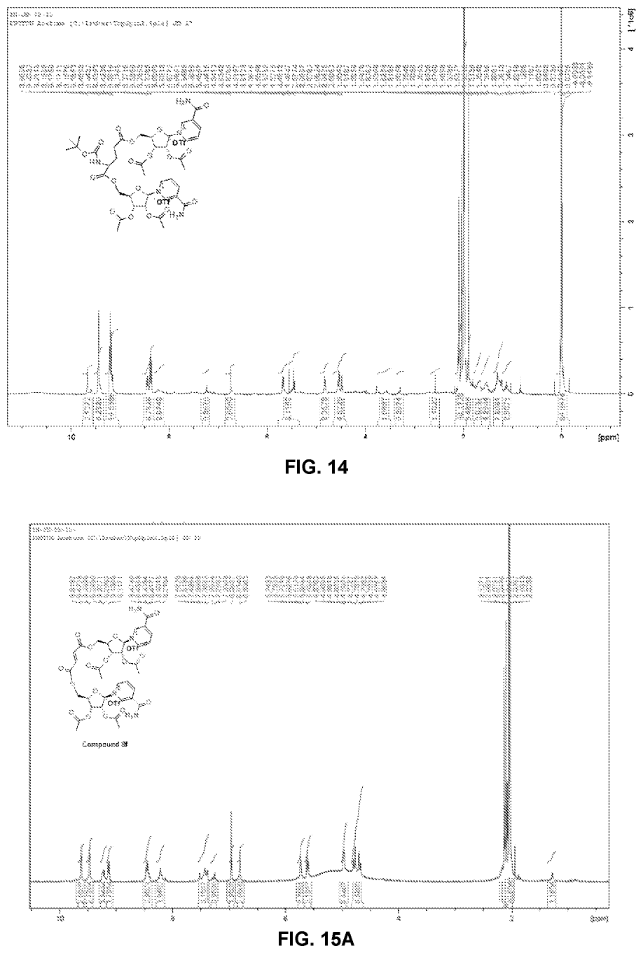

¹H NMR (400 MHz Acetone-d6) ppm: 1.89 (s, 9H), 1.97 (s, 12H, overlapped), 4.46 (dd, J=12.9 Hz, J=2.5 Hz), 4.46 (dd, J=12.9 Hz, J=2.5 Hz), 4.54 (m, 1H), 4.55 (dd, J=12.9 Hz, J=3.2 Hz), 5.67 (dd, J=5.4 Hz, J=3.6 Hz), 6.74 (d, 2H, J=3.5 Hz), 8.44 (t, 2H, J=14 Hz), 9.15-9.17 (brm, 2H), 9.43 (m, 2H), 9.66 (brs, 2H). Exemplary spectrum is shown in FIG. 14.

### Synthesis of Compound 8f.

A ball milling jar was charged with intermediate 8f (70 mg, 0.11 mmoles) and compound 7 (43 mg, 0.22 mmoles), followed by the addition of trimethylsilyltrifluoromethane sulfonate (67 μL, 0.44 mmoles) and 1-2 drops of anhydrous dichloromethane. The resulting mixture was subjected to ball milling conditions on a Retsch MM400 miller for 30 min at 30 Hz. After 30 min, reaction mixture was allowed to cool to room temperature and then it was dissolved into a flask using acetone. The resulting solution was distilled off under reduced pressure, triturated with diethyl ether and dried to obtain compound 8f a yellow foamy and highly hygroscopic solid.

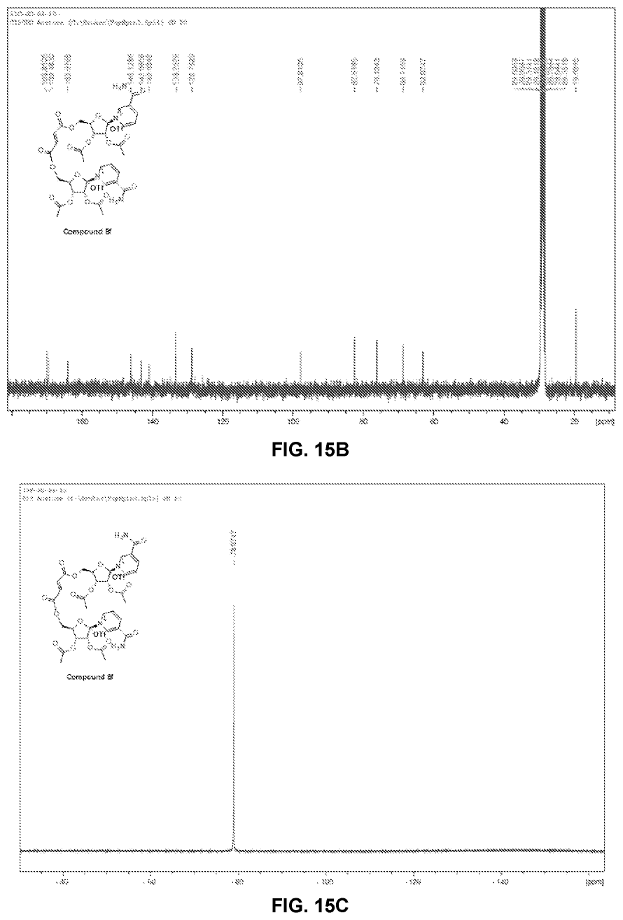

¹H NMR (400 MHz, Acetone-d6) ppm: 2.09 (s, 6H), 2.12 (s, 6H), 4.66-4.8 (dd, 4H, J=2.92 Hz, 2.04 Hz), 4.96-4.97 (m, 2H), 5.61 (t, 2H, J=5.76 Hz), 5.73 (m, 2H), 6.81 (d, 2H, J=3.08 Hz), 6.96 (s, 2H), 8.41-8.47 (m, 2H), 9.12 (m, 2H), 9.46 (m, 2H), 9.61 (s, 2H). ¹³C NMR (100 MHz, Acetone-d6) ppm: 19.52, 62.97, 68.71, 76.12, 82.51, 97.81, 128.75, 133.28, 140.86, 143.06, 146.12, 163.97, 169.48, 169.85. ¹⁹F (376 MHz, Acetone-d6) ppm: −78.97. Exemplary spectra are shown in FIGS. 15A-15C.

### Synthesis of Compound 8g (Protocol 1).

A ball milling jar was charged with intermediate 6g (70 mg, 0.15 mmoles) and compound 7 (33 mg, 0.16 mmoles), followed by the addition of trimethylsilyltrifluoromethane sulfonate (42 μL, 0.22 mmoles). The resulting mixture was subjected to ball milling conditions on a Retsch MM400 miller for 30 min at 30 Hz. After 30 min, reaction mixture was allowed to cool to room temperature and then it was dissolved into a flask using acetone. The resulting solution was distilled off under reduced pressure, triturated with diethyl ether and dried to obtain compound 8g as a highly hygroscopic solid.

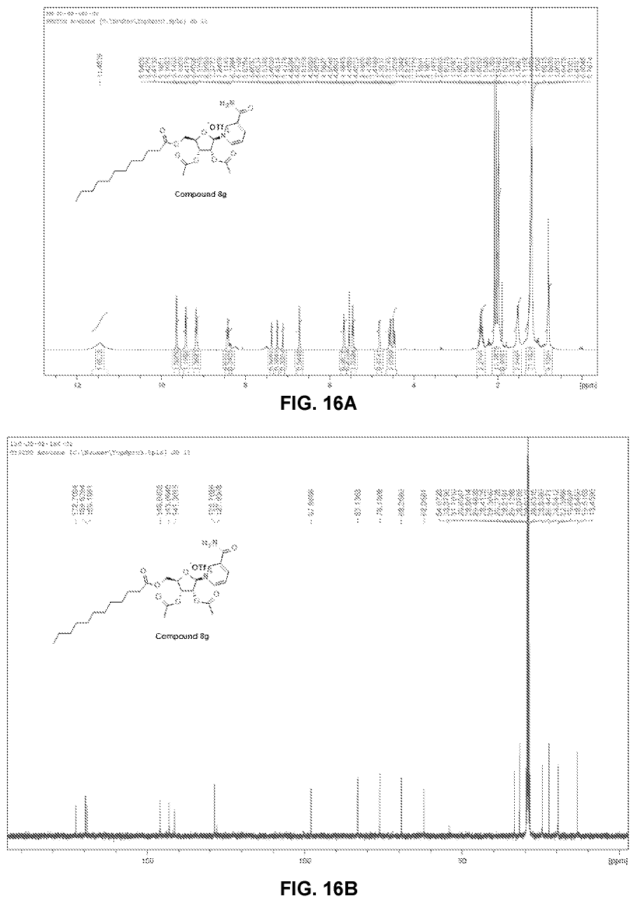

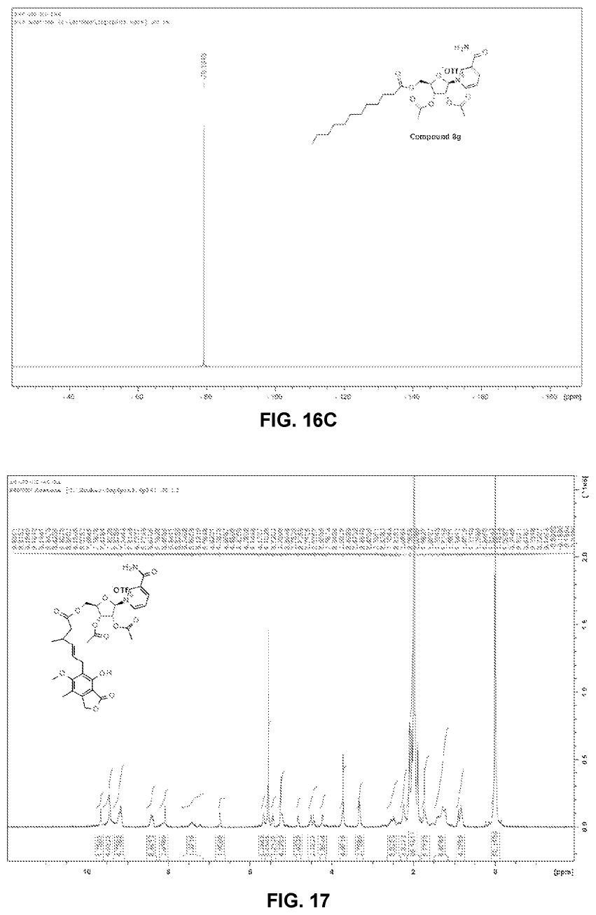

¹H NMR (400 MHz, Acetone-d6) ppm: 0.80 (t, 3H), 1.12 (m, 18H), 1.50-1.55 (m, 2H), 2.04 (s, 3H), 2.08 (s, 3H), 2.33-2.45 (m, 2H), 4.45-4.59 (dd, 2H, J=3.22 Hz, J=2.28 Hz), 4.81 (m, 1H), 5.45 (t, 1H J=5.44 Hz), 5.65-5.67 (m, 1H), 6.72 (s, 1H, J=3.68 Hz), 8.40-8.43 (m, 1H), 9.14-9.18 (m, 1H), 9.41-9.42 (m, 1H), 9.64 (s, 1H). ¹³C NMR (100 MHz, CDCl₃) ppm: −13.45, 19.51, 19.54, 19.66, 22.39, 24.54, 29.07, 29.21, 29.27, 29.3, 29.46, 29.65, 31.7, 33.37, 54.07, 62.05, 69.26, 76.18, 83.13, 97.98, 127.93, 128.7, 141.3, 143.07, 146.04, 169.19, 169.62, 172.7. ¹⁹F (376 MHz, Acetone-d6) ppm: −79.13. Exemplary spectra are shown in FIGS. 16A-16C.

### Synthesis of Compound 8g (Protocol 2).

A ball milling jar was charged with corresponding intermediates 6 g (1 equiv.), and NAM-TMS (compound 7, (1 equiv.), followed by the addition of trimethylsilyltrifluoromethane sulfonate (2 equiv.), and 1-2 drops of anhydrous dichloromethane. The resulting mixture was subjected to ball milling conditions on a Retsch MM400 miller for 30 min at 30 Hz. Reaction progress was monitored by ¹H NMR. After the reaction was completed, reaction mixture was allowed to cool to room temperature and then it was dissolved into a flask using acetone. The resulting solution was distilled off under reduced pressure and triturated with diethyl ether and dried on high vacuum to obtain a yellow foamy and highly hygroscopic mixture.

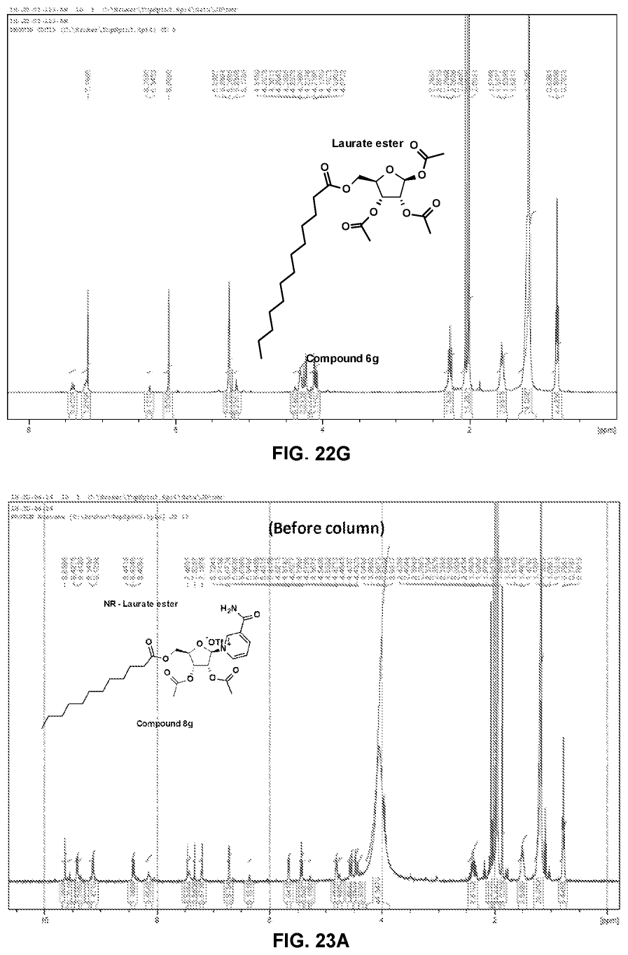

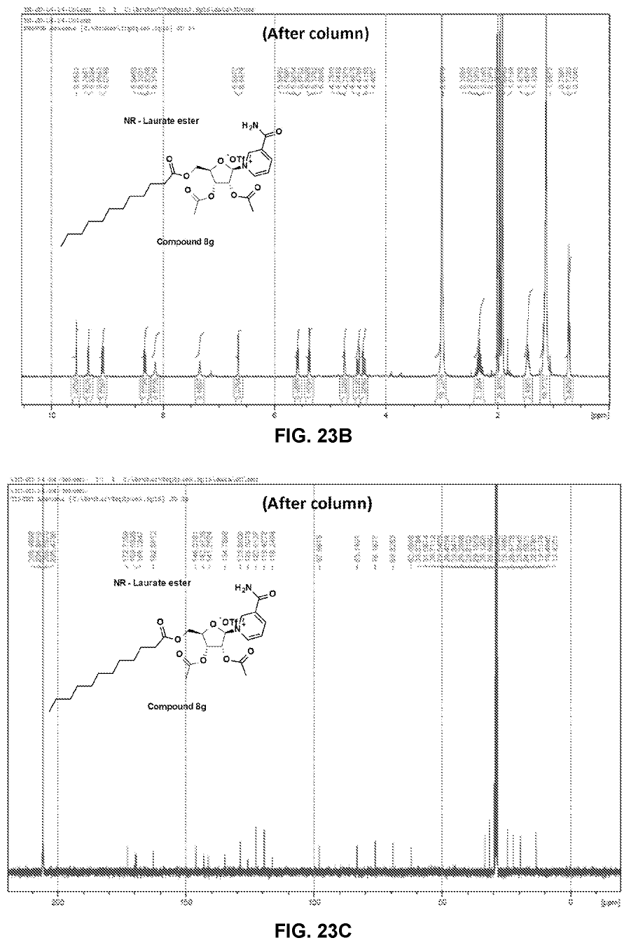

Exemplary ¹H NMR spectra of the product before column chromatography is shown in FIG. 23A. Exemplary ¹H NMR and ¹³C NMR spectra of the product following column chromatography are shown in FIGS. 23B and 23C, respectively.

NR-laurate (NRLR) triflate was successfully purified on a few milligram scale (30-40 mg) on a normal silica column using a mixture of dichloromethane (DCM) and acetone. Pure compound (NRLR) was obtained in 1:1 mixture of DCM and acetone (20 mg, yield: 60%).

1H NMR (400 MHz, Acetone-d6) ppm: 9.55 (s, 1H), 9.32 (d, 1H, J=6.2 Hz), 9.08 (d, 1H, J=8.1 Hz), 8.33 (dd, 1H, J=14.2 Hz, 1.3 Hz), 8.2 (s, 1H, —NH), 7.3 (s, 1H, —NH), 6.55 (d, 1H, J=3.9 Hz), 5.58 (1H, dd, J=4 Hz), 5.37 (t, 1H, J=5.4 Hz), 4.73-4.75 (br m, 1H), 4.40-4.48 (1H, dd, J=3 Hz, 25 Hz), 2.29-233 (m, 2H), 2.07 (s, 3H), 1.96 (s, 3H), 1.43-1.47 (m, 2H), 1.12 (m, 16H), 0.72 (t, 3H, J=6.7 Hz); ¹³C NMR (400 MHz, Acetone-d6) ppm: 13.42, 19.49, 19.51, 22.37, 31.68, 33.37, 62.09, 69.32, 76.16, 83.14, 97.96, 128.68, 134.76, 141.25, 142.97, 146.03, 162.68, 169.22, 169.63, 172.73.

Column purification (teledyne) on a large scale (>50 mg) always led to a mixture of partial deacetylation (2′ or 3′ or both 2′ and 3′) products.

### Synthesis of Compound 8h.

A ball milling jar was charged with intermediate 6h (50 mg, 0.086 mmoles) and compound 7 (17 mg, 0.086 mmoles), followed by the addition of trimethylsilyltrifluoromethane sulfonate (32 μL, 0.172 mmoles). The resulting mixture was subjected to ball milling conditions on a Retsch MM400 miller for 25 min at 30 Hz. After 25 min, reaction mixture was allowed to cool to room temperature and then it was dissolved into a flask using acetone. The resulting solution was distilled off under reduced pressure, triturated with diethyl ether and dried to obtain compound 8h a highly hygroscopic solid.

¹H NMR (400 MHz, Acetone-d6) ppm: 1.7 (s, 3H), 2.03 (s, 3H), 2.08 (s, 3H), 2.09 (s, 3H), 2.3 (m, 2H), 2.43-2.58 (m, 2H), 3.30-3.32 (m, 2H), 3.72 (s, 3H), 4.1-4.44 (m, 1H), 4.45-4.48 (dd, 1H), 4.59-4.62 (dd, 1H), 4.82 (m, 1H), 5.20-5.24 (m, 2H), 5.45 (s, 2H), 5.67 (brs, 1H), 6.73 (brs, 1H), 8.07 (m, 1H), 8.38-8.44 (m, 1H), 9.14-9.18 (m, 1H), 9.43 (brs, −2NHs), 9.65 (s, 1H). Exemplary spectrum is shown in FIG. 17.

### Example 4. Synthesis of Compound 9b

An exemplary synthesis method is provided in this example for compound 9b of Synthesis Scheme 1 as shown in Example 1:

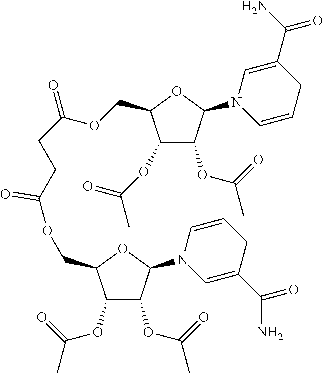 [SMILES: CC(=O)O[C@@H]1[C@H](OC(C)=O)[C@@H](COC(=O)CCC(=O)OC[C@H]2O[C@@H](N3C=CCC(C(N)=O)=C3)[C@H](OC(C)=O)[C@@H]2OC(C)=O)O[C@H]1N1C=CCC(C(N)=O)=C1]

A clean dry round bottomed flask was charged with compound 8b under nitrogen followed by degassed DI water. To this solution, NaHCO₃ was added followed by portion wise addition of Na₂S₂O₄ and stirred for 1-2 min. This was followed by the addition of degassed ethyl acetate and the resulting mixture was stirred at room temperature for 4 h followed by an aqueous work-up using DI water and ethyl acetate. The aqueous layer was extracted twice with degassed ethyl acetate and organic layers were combined, dried and evaporated under reduced pressure to yield the desired compound.

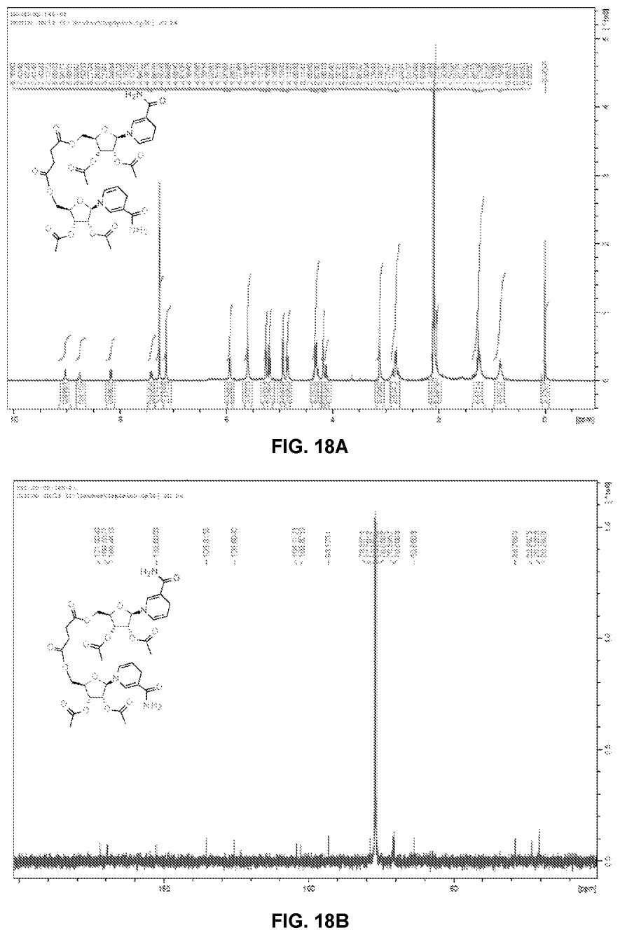

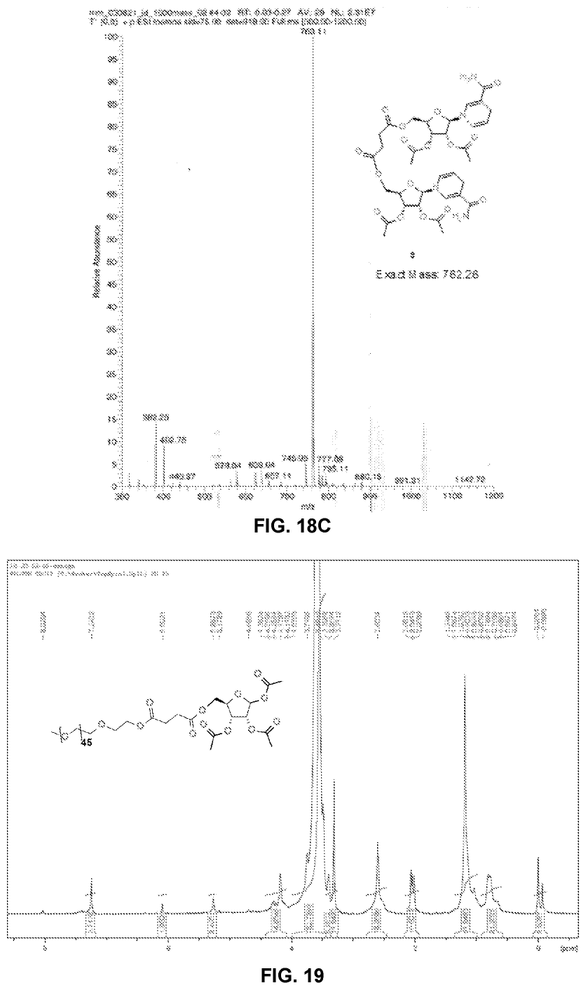

¹H NMR (400 MHz, CDCl₃) ppm: 7.13 (2H, s), 5.24-5.27 (m, 2H), 5.18 (t, 2H, J=12.6 Hz), 5.94 (dd, 2H, J=1.3, 8.1 Hz), 5.6 (brs, 2H, NH2), 4.93 (d, 2H, J=6.9 Hz), 4.83-4.86 (m, 2H), 4.27-4.36 (dd, 4H, J=4.9 Hz, 11.9 Hz), 4.17 (m, 2H), 3.11 (brs, 4H), 2.76-2.87 (brm, 4H), 2.10 (s, 6H, 3-OAc), 2.08 (s, 6H, 3-OAc). ¹³C NMR (100 MHz, CDCl₃) ppm: 20.3, 20.5, 22.9, 28.7, 63.6, 70.6, 70.8, 78.8, 93.1, 102.8, 104.1, 125.6, 135.3, 152.6, 169.5, 171.9; MS: found m/z=763.11 (M+1). Calculated for C₃₄H₄₂N₄O₁₆: 763.2674 (M+1). Exemplary spectra are shown in FIGS. 18A-18C.

### Example 5. Synthesis of Compounds 10(a-h)

Exemplary synthesis methods are provided in this example for compounds 10(a-h) of Synthesis Scheme 1 as shown in Example 1. The structures of compounds 10(a-h) are shown below.

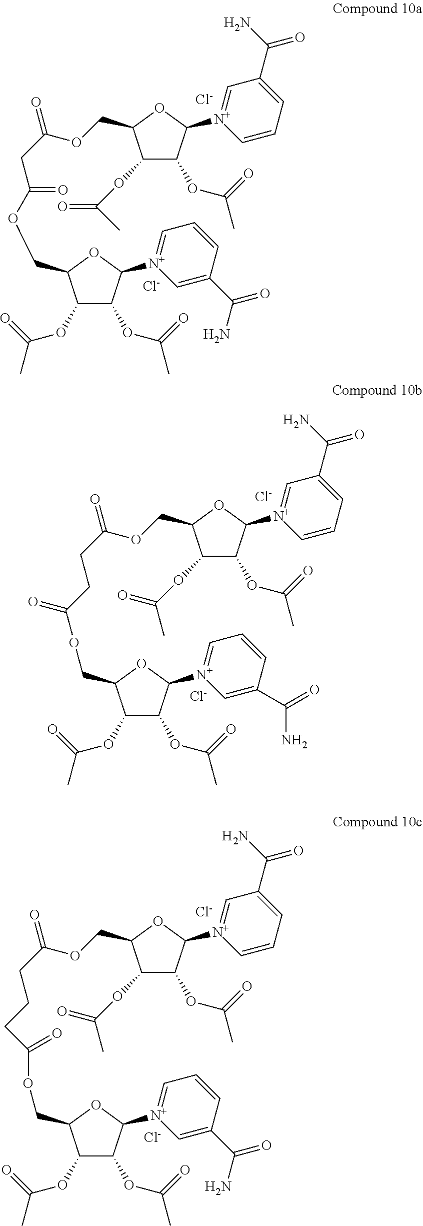

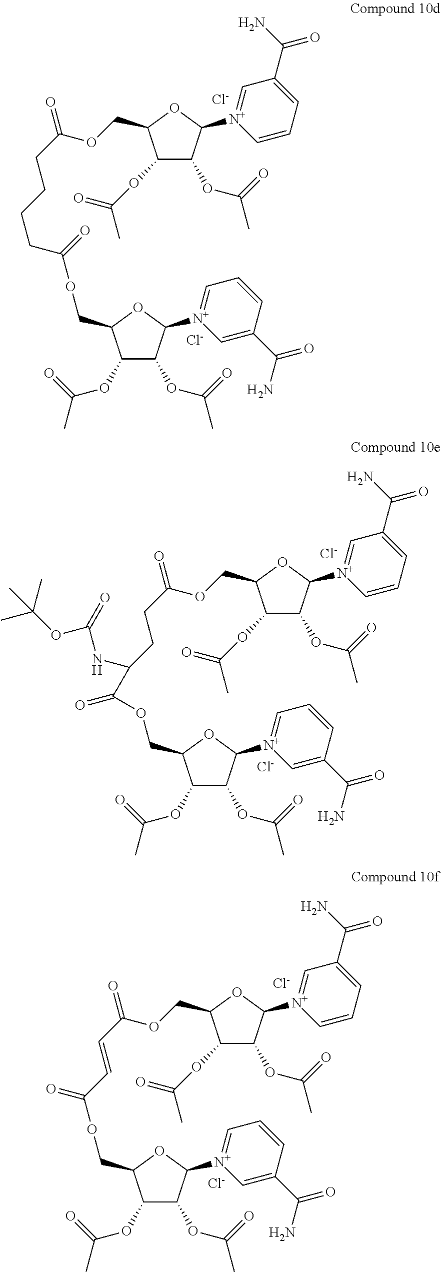

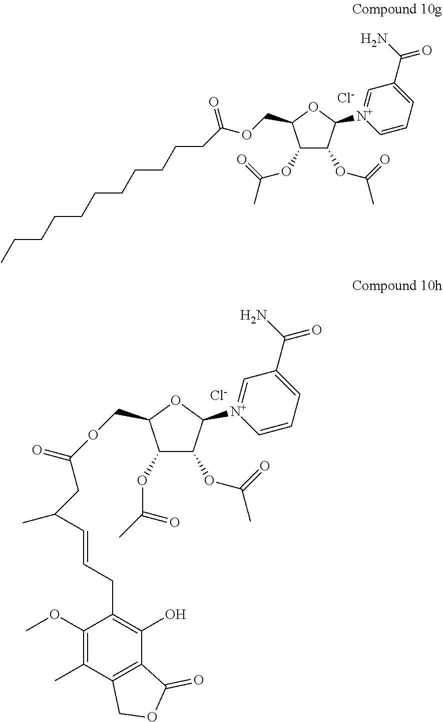 [SMILES: CCCCCCCCCCCC(=O)OC[C@H]1O[C@@H]([n+]2cccc(C(N)=O)c2)[C@H](OC(C)=O)[C@@H]1OC(C)=O ; [Cl-] ; COc1c(C)c2c(c(O)c1C/C=C/C(C)CC(=O)OC[C@H]1O[C@@H]([n+]3cccc(C(N)=O)c3)[C@H](OC(C)=O)[C@@H]1OC(C)=O)C(=O)OC2 ; [Cl-]]

In a clean round bottom flask NR-laurate (NRLR) triflate was dissolved in a 1:1 mixture of water and acetonitrile and the resulting mixture was cooled over ice to 0-5° C. and stirred for 15-20 min at the same temperature. To this ice-cold solution, amberlite resin was added and stirred at 0-5° C. for 2 hours. A glass column was packed with amberlite resin and the column was equilibrated with DI water. The above mixture NRLR triflate with the amberlite resin was added to the column and eluted using 1:1 mixture of water and acetonitrile.

Collected fractions were combined and evaporated under reduced pressure to yield a white powder of NRLR chloride free of triflate ions. The resulting product was analyzed by ¹H NMR and ¹⁹F NMR to confirm the ion exchange of triflate to chloride. The absence of fluorine peak in ¹⁹F NMR confirmed the complete conversion of corresponding triflate salt to chloride and ¹H NMR revealed the desired peaks of NRLR-chloride with a slight shift in the chemical shifts relative to NRLR-triflate which was expected due to ion-exchange.

Reaction conditions and ion-exchange chromatography were optimized so that the obtained compound is of desired purity. Conditions were modified and tuned carefully to circumvent the column chromatography which otherwise was leading to deacetylation impurities.

Synthesis of Compound 10g from Crude Compound 8g

Crude compound 8g was obtained as described in Example 3, Synthesis of compound 8g (protocol 2). The obtained crude compound was converted to a chloride salt by amberlite exchange chromatography, and the resulting chloride derivative (compound 10g) was purified by reverse phase C18 column chromatography on teledyne. Desired fractions were collected, evaporated, dried and analyzed by ¹HNMR and ¹³CNMR. Compound 10g was found to be pure and free of nicotinamide (NAM) or other impurity traces.

It was found that purification by column chromatography can be circumvented using certain reaction conditions. The protocol for synthesis of compound 10g without column purification is as follows:

A ball milling jar was charged with laurate ester intermediate 6g (100 mg, 0.21 mmoles, 1 equiv.), and NAM-TMS (compound 7) (41 mg, 0.21 mmoles, 1 equiv.), followed by the addition of trimethylsilyltrifluoromethane sulfonate (76 μL, 0.42 mmoles, 2 equiv.), and 1-2 drops of anhydrous dichloromethane. The resulting mixture was subjected to ball milling on a Retsch MM400 miller for 30 min at 30 Hz. Reaction progress was monitored by ¹HNMR. After the reaction was completed, reaction mixture was allowed to cool to room temperature and then it was dissolved in to a flask using acetone. The resulting solution was distilled off under reduced pressure and the resulting compound was co-distilled twice with diethyl ether (10-15 mL) and dried on high vacuum to obtain a pale yellow syrup (150 mg, 100% yield). Resulting compound was analyzed by ¹HNMR and was confirmed to be 90-95% pure and free of NAM impurity. This compound was further subjected to amberlite-C ion exchange chromatography without further steps of purification.

1H NMR (400 MHz, D₂O) ppm: 9.36 (s, 1H), 9.1 (d, 1H, J=6.3 Hz), 9.0 (d, 1H, J=8.2 Hz), 8.28 (dd, 1H, J=9.0, 6.1 Hz), 6.59-6.6 (d, 1H, 1′-ribose Hs, J=4.3 Hz), 5.45-5.47 (t, 1H, 2′-ribose Hs, J=5.0 Hz), 5.28-5.31 (t, 1H, 3′-ribose Hs, J=5.0 Hz), 4.73-4.74 (br m, 1H, 4′-ribose Hs), 4.41 (br d, 2H, 5′-ribose Hs), 2.26-2.31 (br m, 2H, —CH₂CO), 2.09 (s, 3H), 1.99 (s, 3H), 1.42-1.43 (br m, 2H), 1.11 (br m, 16H), 0.71 (t, 3H, J=5.9 Hz); ¹³C NMR (400 MHz, D₂O) ppm: 13.74, 19.76, 19.85, 22.48, 24.55, 28.88, 29.24, 29.4, 29.54, 29.56, 31.77, 33.57, 62.71, 69.79, 75.84, 82.59, 97.22, 128.85, 134.33, 140.29, 143.37, 146.31, 164.51, 171.03, 171.29, 173.72; HRMS: Calculated C₂₇H₄₁N₂O₈·Cl: 556.2551; C₂₇H₄₁N₂O₈.: 521.2863; found 521.2909 (M+).

### Ion Exchange Chromatography of NR-Triacetate (NRTA) to NRTA Chloride

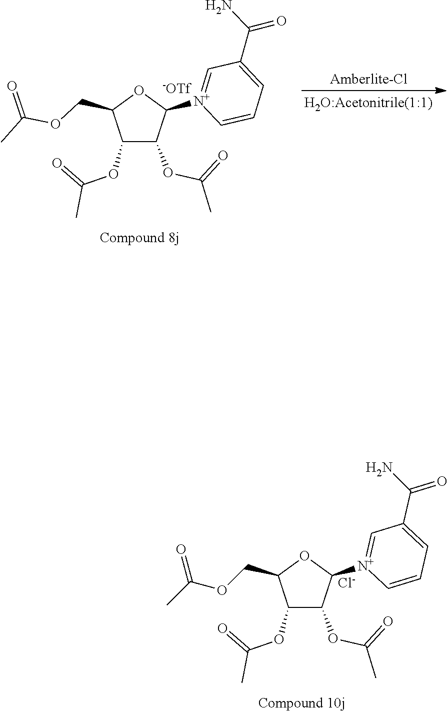 [SMILES: CC(=O)OC[C@H]1O[C@@H]([n+]2cccc(C(N)=O)c2)[C@H](OC(C)=O)[C@@H]1OC(C)=O ; CC(=O)OC[C@H]1O[C@@H]([n+]2cccc(C(N)=O)c2)[C@H](OC(C)=O)[C@@H]1OC(C)=O ; [Cl-] ; "-OTf"]

In a clean round bottom flask with 100 mg of NR-triflate (compound 8j), 1:1 mixture of acetonitrile and water (2:2 mL each) was added and the mixture was cooled over ice for 15 minutes. To the ice-cold mixture, amberlite-Cl resin (1.5 g) was added and reaction mixture was stirred at 0-5 C for 2 h. A glass column was packed with amberlite-Cl resin and equilibrated with 1:1 mixture of acetonitrile and water. The ice cold mixture of compound 8j in amberlite-Cl resin was added to the column and eluted with 1:1 mixture of acetonitrile and water. Resulting fractions were collected, distilled, dried and analyzed by ¹HNMR and ¹⁹FNMR. ¹HNMR revealed NRTA peaks and the disappearance of fluorine peak in ¹⁹FNMR revealed the successful ion exchange. The resulting compound was confirmed as NRTA-Chloride (compound 10j) (Yield: 89%).

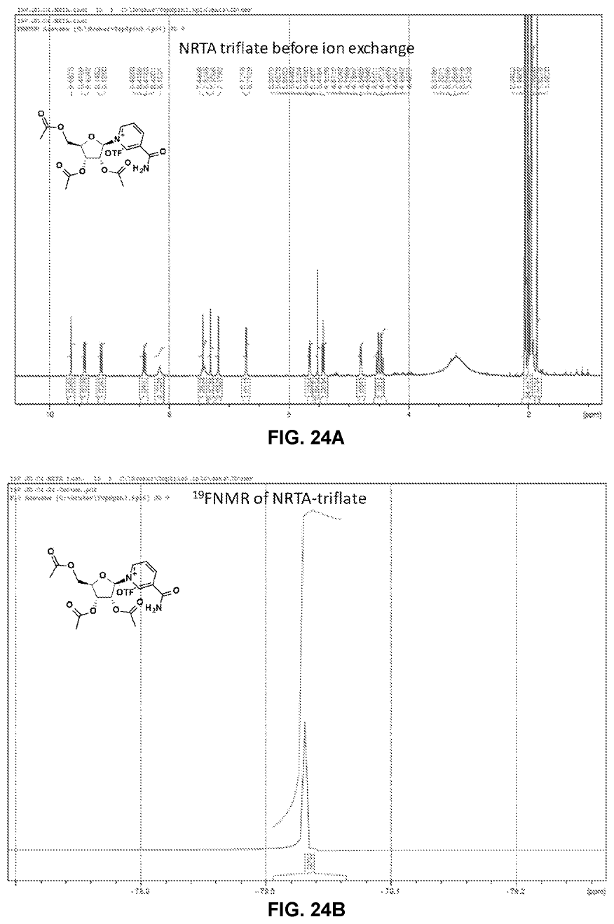

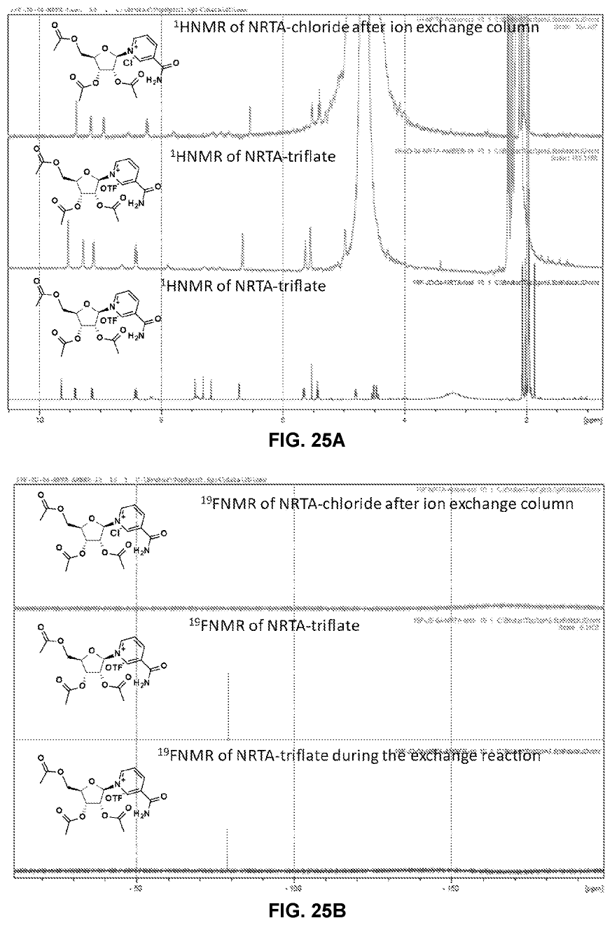

Exemplary ¹H NMR and ¹⁹F NMR spectra of NRTA triflate before ion-exchange are shown in FIGS. 24A and 24B, respectively. FIG. 25A shows exemplary ¹H NMR spectra of NRTA triflate after ion exchange (top panel) compared with the spectra before and during ion exchange (middle and bottom panels, respectively). FIG. 25B shows exemplary ¹⁹F NMR spectra of NRTA triflate after ion exchange (top panel) compared with the spectra before and during ion exchange (middle and bottom panels, respectively).

Ion Exchange Chromatography of Compound 8g to Compound 10g after Column Chromatography

In a clean round bottom flask NR-laurate (NRLR) (compound 8g) (150 mg, 0.22 mmoles) was dissolved in a 1:1 mixture of water and acetonitrile (3 mL each) and the resulting mixture was cooled over ice to 0-5° C. and stirred for 15-20 min. To this ice-cold solution, amberlite resin (1.5 g) was added and stirred at 0-5° C. for 2 hours. A glass column was packed with amberlite-Cl (IR-410) resin (5 g) and column was equilibrated with 1:1 mixture of ACN:H₂O. The ice-cold mixture of NRLR (compound 8g) with amberlite resin was added to the column and eluted using 1:1 mixture of water and acetonitrile (50 mL).

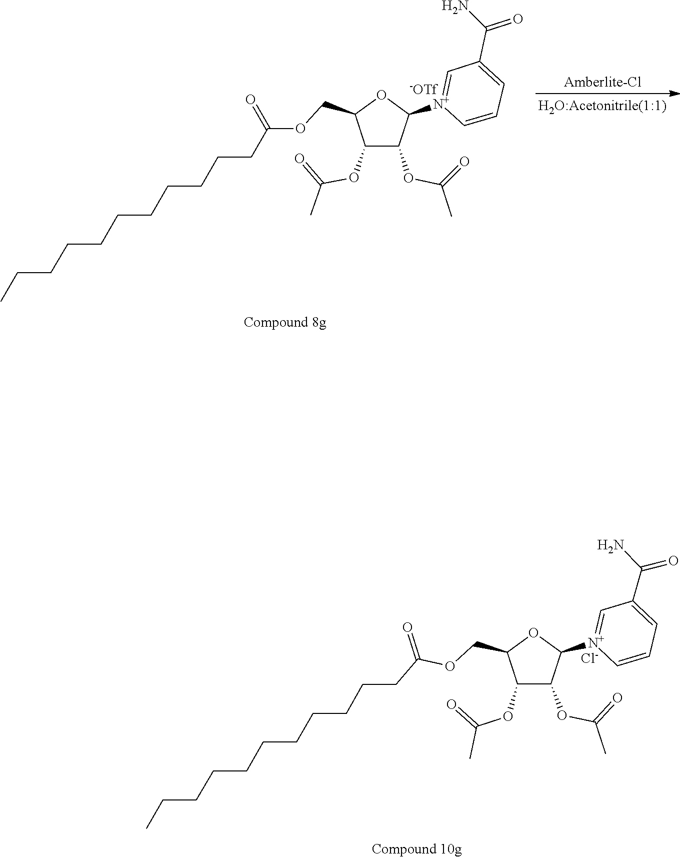 [SMILES: CCCCCCCCCCCC(=O)OC[C@H]1O[C@@H]([n+]2cccc(C(N)=O)c2)[C@H](OC(C)=O)[C@@H]1OC(C)=O ; CCCCCCCCCCCC(=O)OC[C@H]1O[C@@H]([n+]2cccc(C(N)=O)c2)[C@H](OC(C)=O)[C@@H]1OC(C)=O ; [Cl-] ; "-OTf"]

Collected fractions were combined and evaporated under reduced pressure to yield a white powder of NRLR chloride free of triflate ions. The resulting product was analyzed by ¹HNMR and ¹⁹FNMR to confirm the ion exchange of triflate to chloride. The absence of fluorine peak in ¹⁹FNMR confirmed the complete conversion of corresponding triflate salt to chloride and ¹HNMR revealed the desired peaks of NRLR-chloride with a slight shift in the chemical shifts relative to NRLR-triflate which was expected due to ion-exchange. The resulting compound was confirmed as NRLR-Chloride (compound 10g) (Yield: 85%).

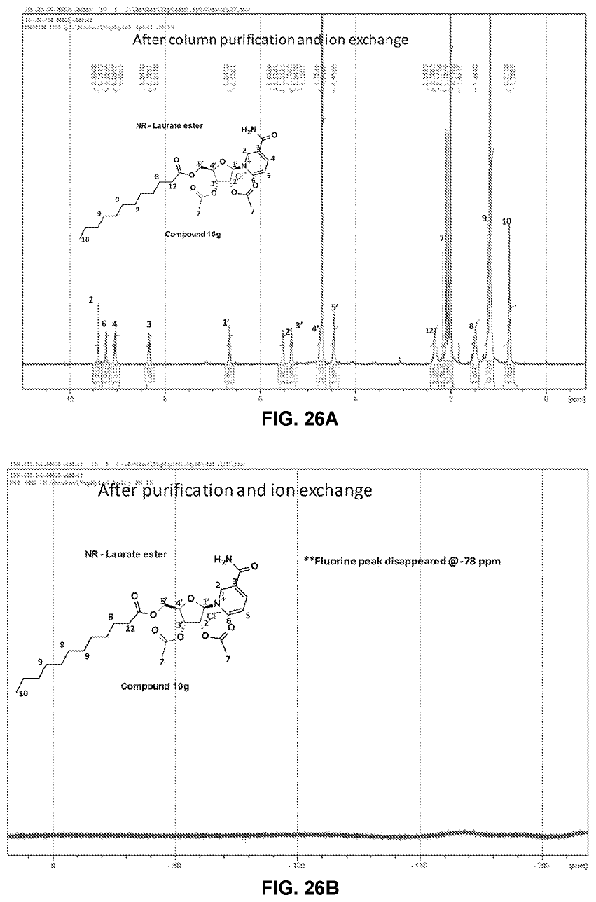

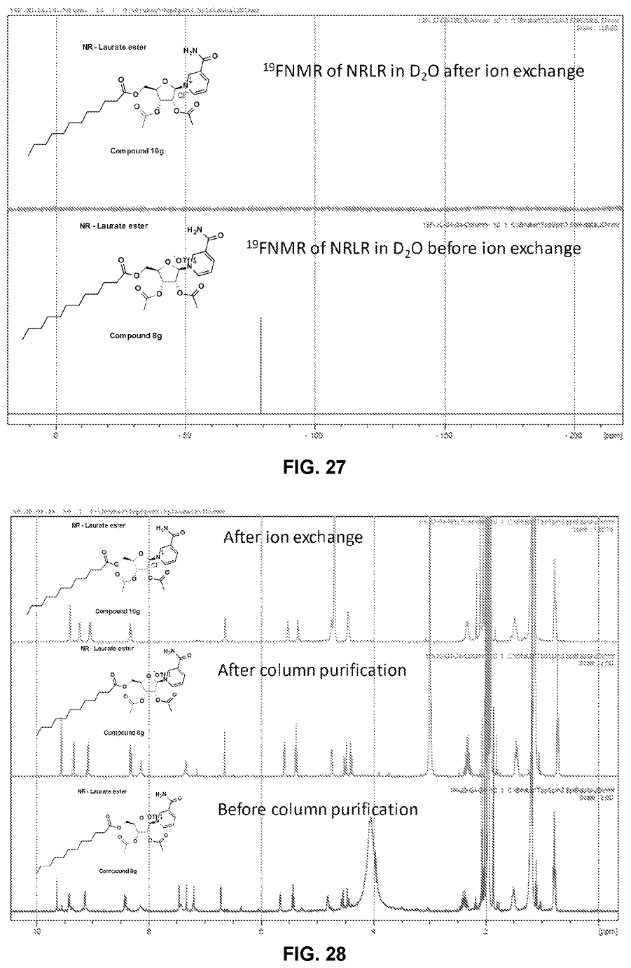

FIGS. 26A and 26B show exemplary ¹H NMR and ¹⁹F NMR spectra, respectively, of compound 10g (NRLR-Cl) following column purification and ion exchange. FIG. 27 shows exemplary ¹⁹F NMR spectra of NRLR in D₂O before (bottom panel) and NRLR-Cl in D₂O after (top panel) ion exchange chromatography. FIG. 28 shows exemplary ¹H NMR spectra of NRLR-Cl after column and ion exchange (top panel), NRLR after column purification (middle panel), and NRLR before column purification (bottom panel).

Ion Exchange Chromatography of Compound 8g to Compound 10g without Column Chromatography

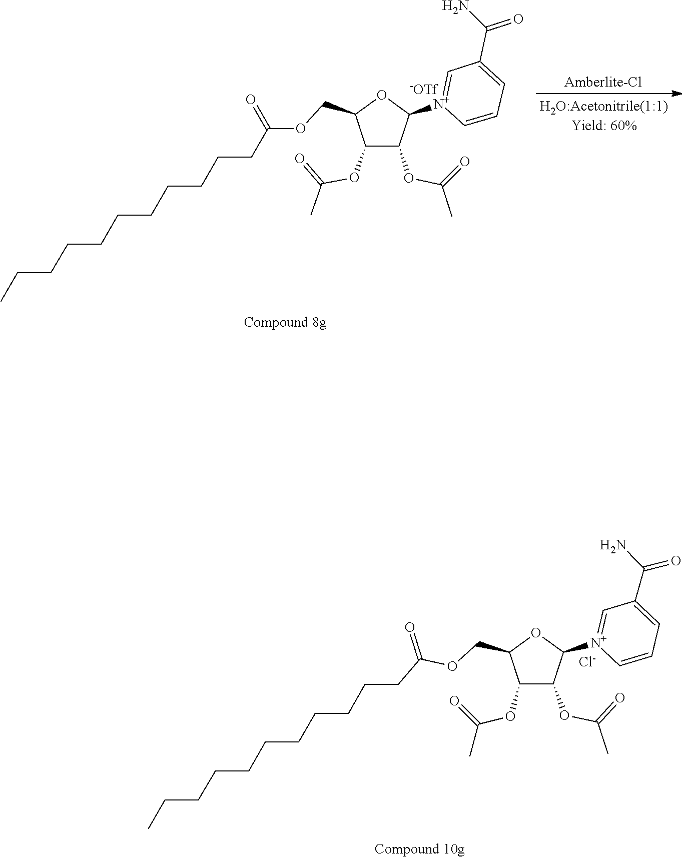 [SMILES: CCCCCCCCCCCC(=O)OC[C@H]1O[C@@H]([n+]2cccc(C(N)=O)c2)[C@H](OC(C)=O)[C@@H]1OC(C)=O ; CCCCCCCCCCCC(=O)OC[C@H]1O[C@@H]([n+]2cccc(C(N)=O)c2)[C@H](OC(C)=O)[C@@H]1OC(C)=O ; [Cl-] ; "-OTf"]

In a clean round bottom flask with 140 mg of NRLR-triflate (compound 8g), 1:1 mixture of acetonitrile and water (3:3 mL each) was added and the mixture was cooled over ice for 15 minutes. To the ice-cold mixture, amberlite-Cl resin (1.5 g) was added and reaction mixture was stirred at 0-5° C. for 2 h. A glass column was packed with amberlite-Cl resin and equilibrated with 1:1 mixture of acetonitrile and water. The ice cold mixture of compound 8g in amberlite-Cl resin was added to the column and eluted with 1:1 mixture of acetonitrile and water. Resulting fractions were collected, distilled, dried and was subjected to ether washings. A white solid was obtained after ether washing which was subsequently dried and analyzed by ¹HNMR and ¹⁹FNMR. ¹HNMR revealed NRTA peaks and the disappearance of fluorine peak in ¹⁹FNMR revealed the successful ion exchange. The resulting compound was confirmed as NRLR-Chloride (compound 10g, yield: 60%).

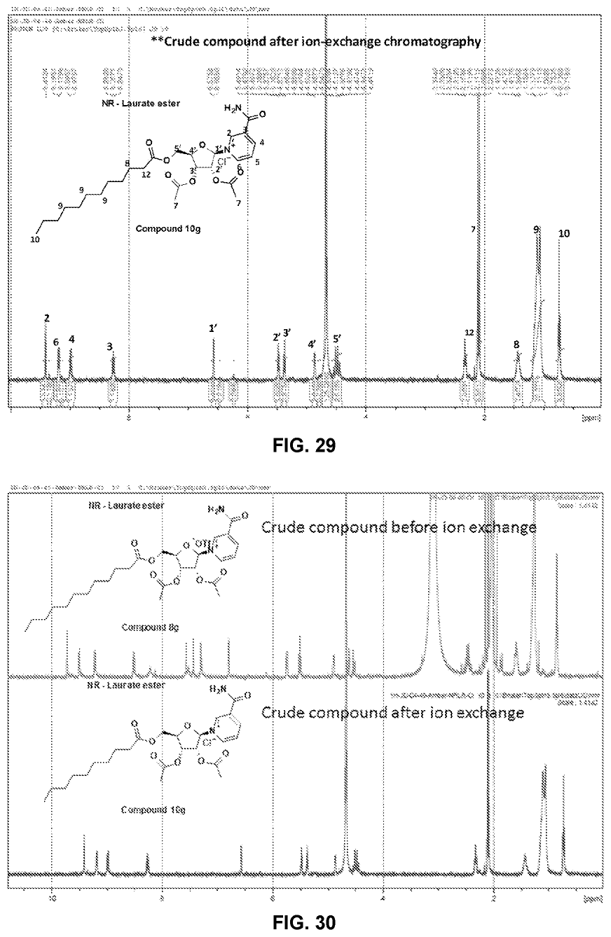

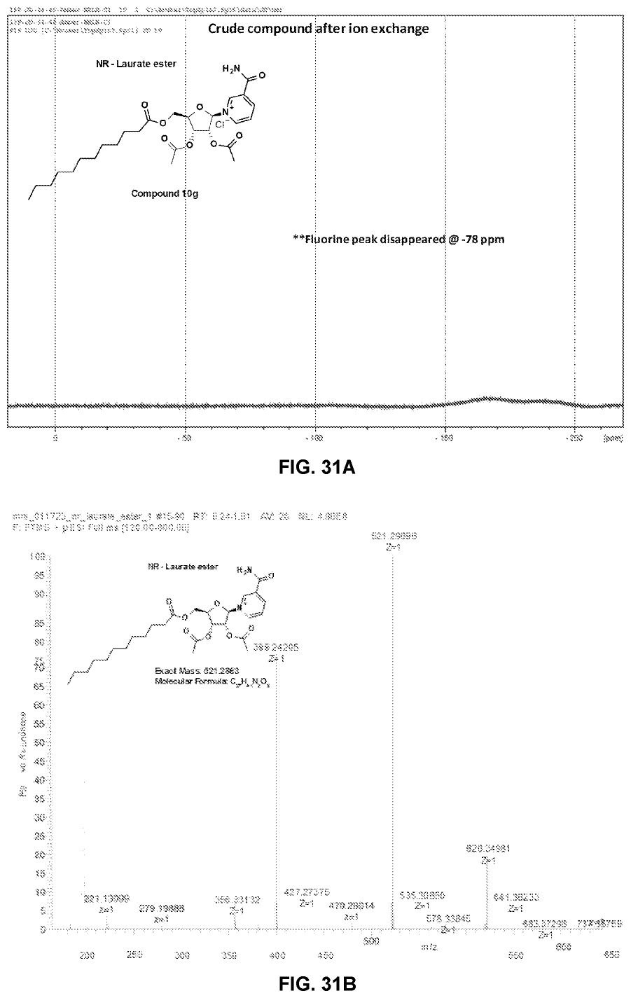

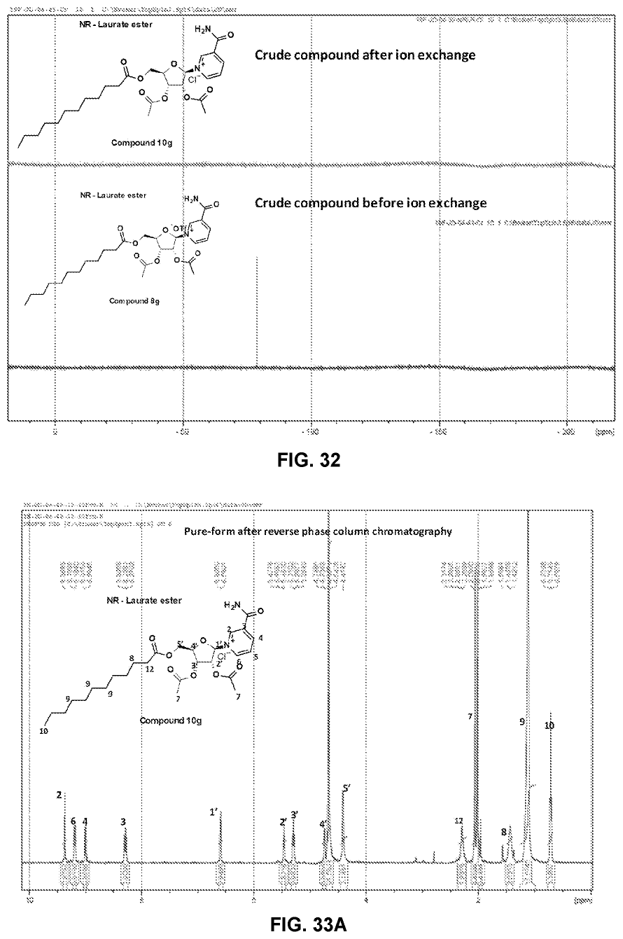

FIG. 29 shows exemplary ¹H NMR spectra of NRLR without column chromatography (“crude NRLR”). FIG. 30 shows exemplary ¹H NMR spectra of crude NRLR before (top panel) and after (bottom panel) ion exchange. FIG. 31A shows exemplary ¹⁹F NMR spectra of crude NRLR after ion exchange. FIG. 31B shows exemplary mass spectra of NRLR. FIG. 32 shows exemplary ¹⁹F NMR spectra of crude NRLR after (top panel) and before (bottom panel) ion exchange. The results in FIGS. 29-32B demonstrate that the chloride salt of NRLR is readily generated from crude compound 8g without column chromatography by using amberlite as an anion exchange and purification process.

### Reverse Phase Column Chromatography of Compound 10g

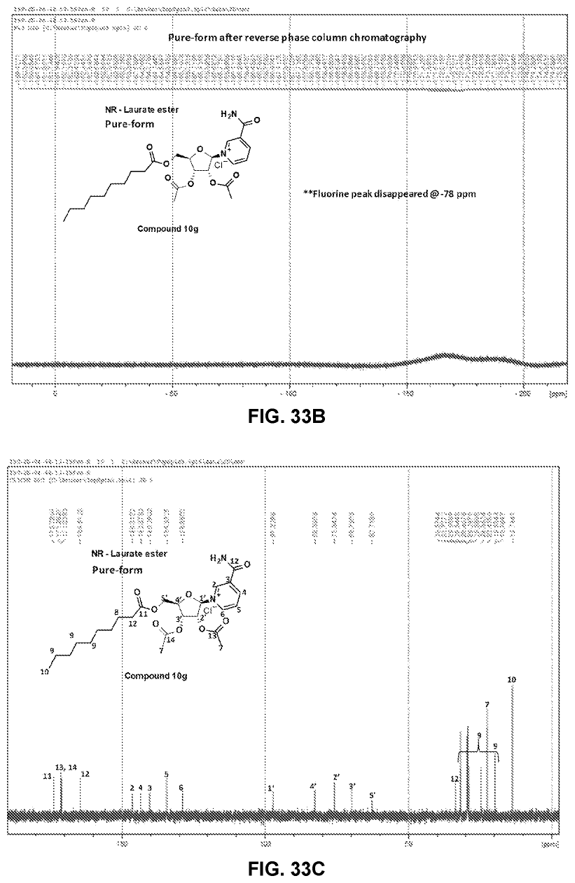

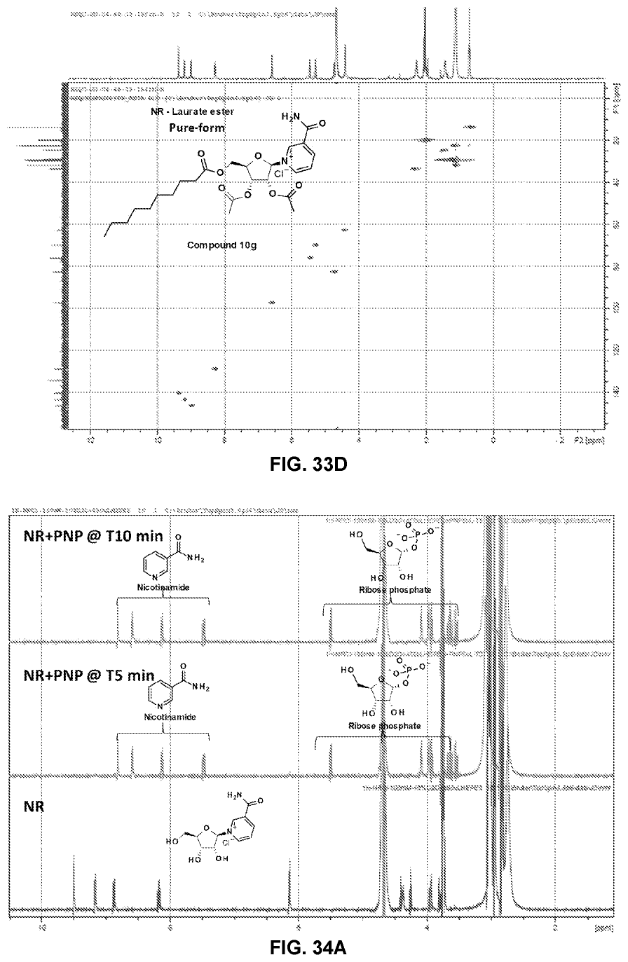

Crude compound (NRLR-OTf, compound 8g with ˜80% purity by NMR) obtained from Vörbruggen reaction was subjected to ion-exchange chromatography to yield compound 10g. Compound 10g (with ˜80% purity, 500 mg) was purified by reverse phase C18 column chromatography on Teledyne using a mixture of acetonitrile and water to obtain pure compound 10g (NRLR-Cl) as a white solid (250 mg, 50% yield). Desired compound was eluted in 25-30% acetonitrile in water. FIGS. 33A, 33B, 33C, and 33D show exemplary ¹H NMR, ¹⁹F NMR, ¹³C NMR, and HSQC spectra, respectively, of compound 10g after the reverse phase column chromatography.

### Example 6. Synthetic Scheme 2

Exemplary synthesis methods are provided in this example for Synthetic Scheme 2 below.

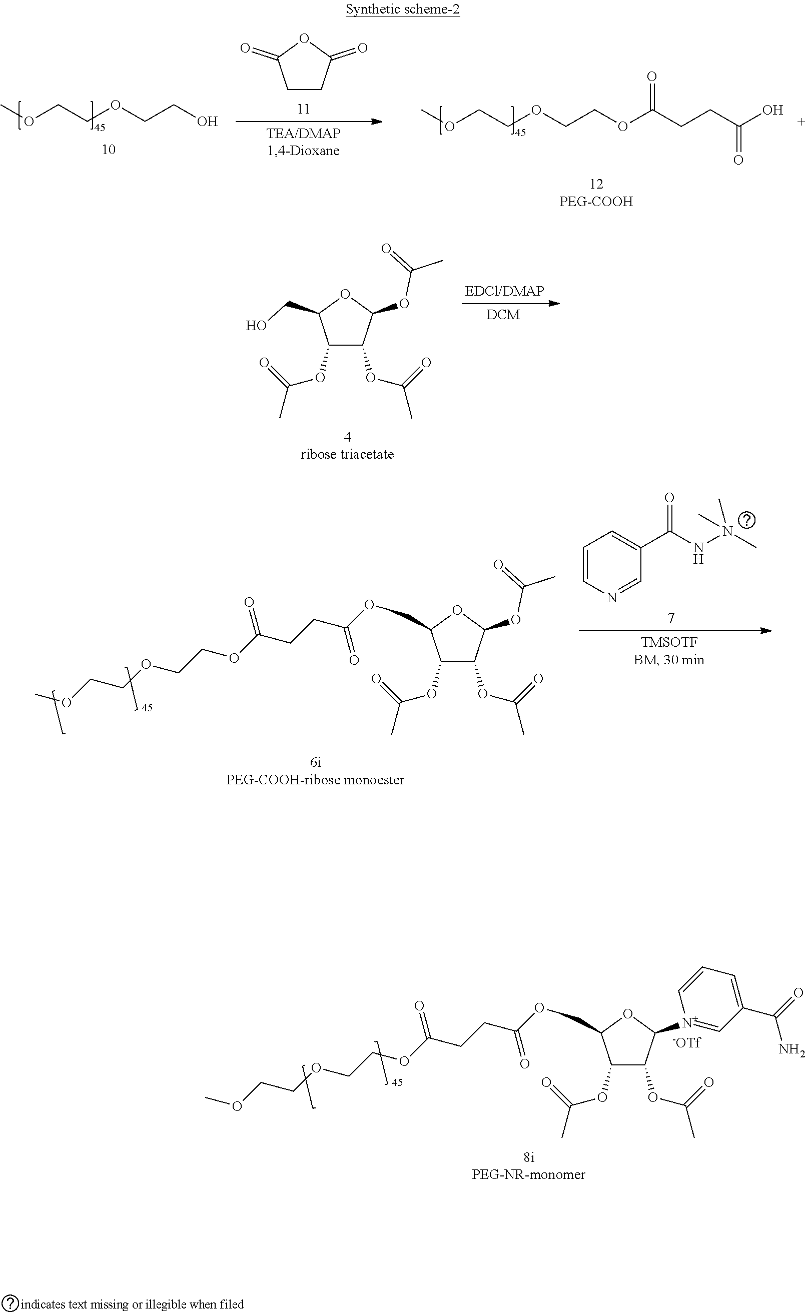

### Synthesis of Compound 12.

A clean dry ball milling jar was charged with mono-functionalized PEG-2000, followed by succinic anhydride, DMAP, trimethylamine and 1-2 drops of 1,4-dioxane. The contents were subjected to ball milling conditions on a Retsch MM400 miller for 30 min at 30 Hz. After 30 min, reaction mixture was allowed to cool to room temperature and dissolved in 1,4-dioxane. The resulting solution was precipitated by diethyl ether to obtain a white precipitate which was dried and taken to the next step without further characterization.

A solution-based reaction (i.e., in a reaction vessel instead of ball milling) was also performed and reproduced the same results. Proton NMR was indistinguishable with both reaction formats.

¹H NMR (400 MHz, CDCl₃) ppm: 2.61-2.68 (m, 4H), 3.38 (s, 3H), 3.46 (t, 2H, J=4.6 Hz), 3.53-3.56 (m, 4H), 3.6-3.73 (br m, −180H), 3.83 (m, 2H), 4.26 (m, 2H). ¹³C NMR (100 MHz, CDCl₃) ppm: 29.6, 31.9, 39.6, 58.9, 63.6, 68.9, 70.4, 71.0, 106.4, 141.9, 175.0, 191.7.

### Synthesis of Compound 6i.

A clean dry ball milling jar was charged with compound 12, followed by compound 4, EDCI, DMAP and 1-2 drops of anhydrous dichloromethane. The resulting mixture was subjected to ball milling conditions on a Retsch MM400 miller for 90 min at 30 Hz. After 90 min, reaction mixture was dissolved in dichloromethane into a flask and evaporated under reduced pressure to yield a white powder. A downfield shift of 5′Hs of compound 4 indicated the successful conjugation.

A solution-based reaction (i.e., in a reaction vessel instead of ball milling) was also performed and reproduced the same results. Proton NMR was indistinguishable with both reaction formats.

¹H NMR (400 MHz, CDCl₃) ppm: 2.08 (s, 3H), 2.11 (s, 3H), 2.13 (s, 3H), 2.66 (m, 4H), 2.87 (m, 2H), 3.38 (s, 3H), 3.46 (m, 2H), 3.6-3.8 (br m, −180H), 4.2 (m, 1H), 4.25 (m, 2H), 4.35 (m, 1H), 5.32 (m, 2H), 6.15 (br s, 1H). ¹³C NMR (100 MHz, CDCl₃) ppm: 20.4, 21.05, 28.8, 59.02, 61.6, 63.8, 63.9, 63.99, 69.0, 70.2, 70.3 (very intense, indicative of PEG carbons), 70.4, 71.9, 72.7, 74.1, 98.1. An exemplary spectrum is shown in FIG. 19.

### Synthesis of Compound 8i.

A ball milling jar was charged with intermediate 13 (53 mg, 0.02 mmoles) and compound 7 (4.2 mg, 0.02 mmoles), followed by the addition of trimethylsilyltrifluoromethane sulfonate (9 μL, 0.03 mmoles) and 1-2 drops of anhydrous dichloromethane. The resulting mixture was subjected to ball milling conditions on a Retsch MM400 miller for 30 min at 30 Hz. After 30 min, reaction mixture was allowed to cool to room temperature and then it was dissolved into a flask using acetone. The resulting solution was distilled off under reduced pressure and dried to obtain compound 8i an off-white hygroscopic solid. Product was confirmed by NMR.

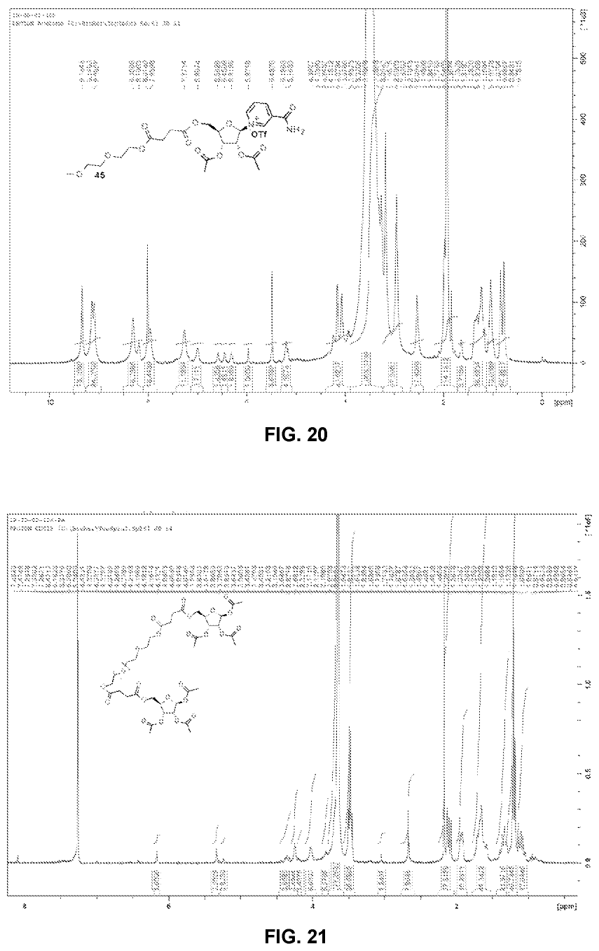

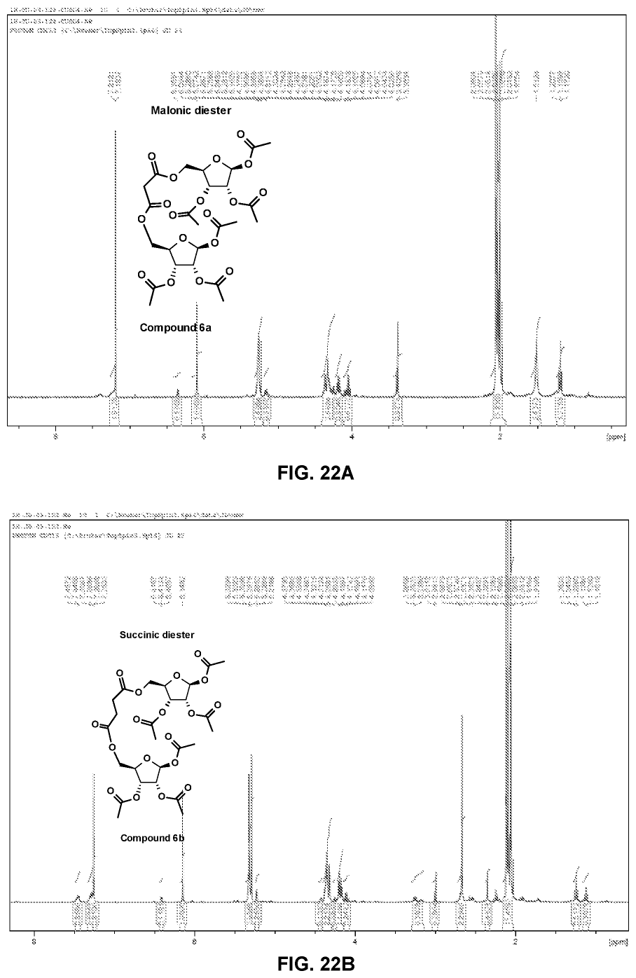

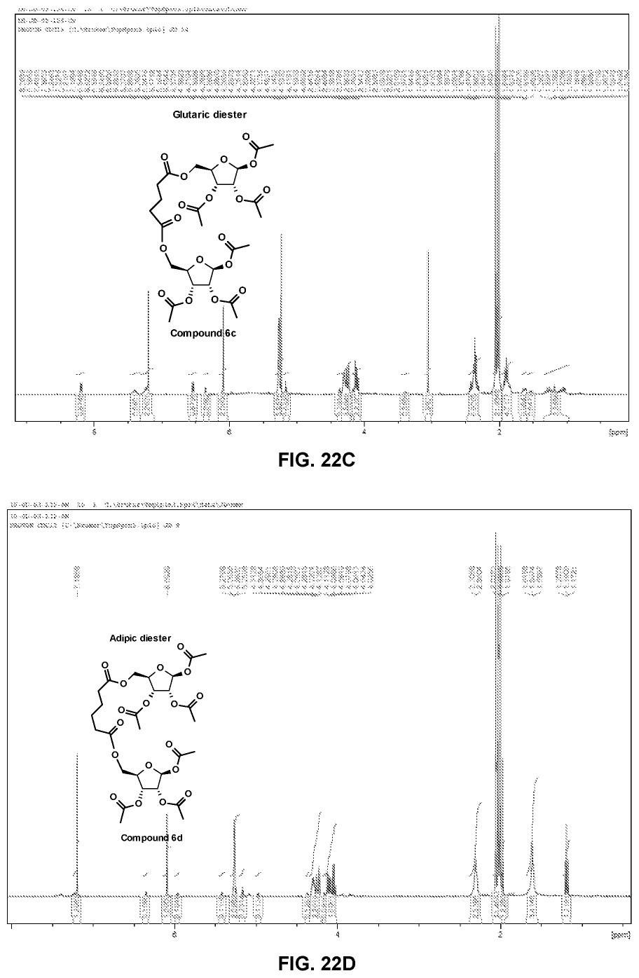

¹H NMR (400 MHz, Acetone-d6) ppm: 2.08 (s, 6H), 2.53 (s, 3H), 3.26-3.7 (m, cluster H's, polymer), 3.8-4.9 (m, polymer Hs ˜180H, 4′H, 3′H), 5.16-5.19 (m, 2H), 5.97 (s, 1H), 8.01 (m, 1H), 8.3 (m, 1H), 9.14 (m, 1H), 9.34 (m, 1H). An exemplary spectrum is shown in FIG. 20.

### Example 7. Synthetic Scheme 3

Exemplary synthesis methods are provided in this example for Synthetic Scheme 3 below.

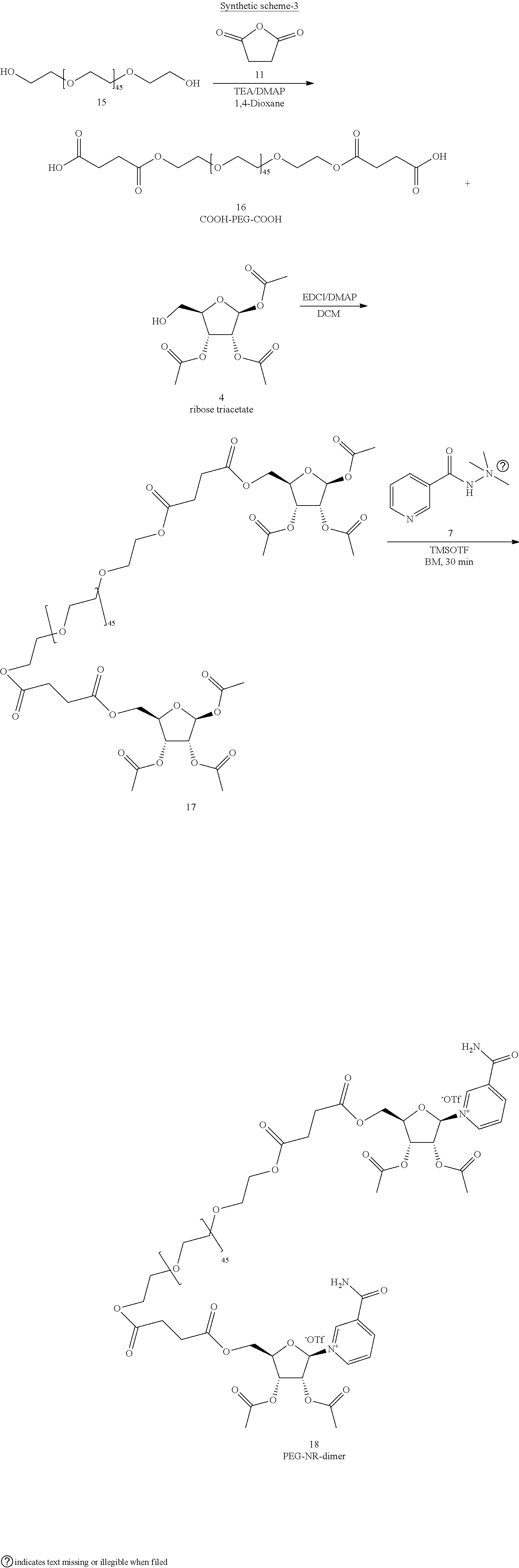

### Synthesis of Compound 16.

To a solution of polyethylene glycol (Mwt: ˜2000, 1 g, 1 equiv., 0.5 mmoles) in 1,4-dioxane (15 ml) was added succinic anhydride (60 mg, 1.1 equiv., 0.6 mmoles), DMAP (61 mg, 1 equiv., 0.5 mmoles) and triethylamine (50 mg, 1 equiv., 0.5 mmoles). The resulting solution was stirred at room temperature under argon for 24 h. Then the desired product was precipitated using diethylether, filtered and dried to obtain a white solid.

¹H NMR (400 MHz, CDCl₃) ppm: 2.62-2.66 (m, 8H), 3.45-3.48 (m, 5H), 3.64-3.73 (m, ˜180H), 3.81 (t, 2H, J=4.5 Hz), 4.26 (t, 2H, J=4.6 Hz).

### Synthesis of Compound 17.

A mixture of PEG-COOH (50 mg, 0.02 mmoles), intermediate 4 (13 mg, 0.04 mmoles), DCC (10 mg, 0.04 mmoles), DMAP (cat.), and 1-2 drops of dichloromethane were charged in a clean and dry ball milling jar. The resulting mixture was subjected to ball milling conditions on a Retsch MM400 miller for 30 min at 30 Hz. The resulting mixture was collected into a flask using DCM and evaporated under reduced pressure to obtain the crude compound.

¹H NMR (400 MHz, CDCl₃) ppm: 2.07-2.17 (s, 18H), 2.67 (m, 8H), 3.5-3.79 (br m, ˜180H), 4.04 (m, 4H), 4.23-4.44 (m, 6H), 5.33 (m, 4H), 6.16 (br s, 2H). An exemplary spectrum is shown in FIG. 21.

### Synthesis of Compound 18.

An analogous reaction as described above for synthesis of compound 8i from compound 6i is performed to obtain compound 18 from compound 17.

### Example 8. NAD Repletion Study

Exemplary studies for testing whether the compounds described herein are capable of contributing to a cell's NAD pool are provided.

Part I: Evaluation of the compounds as precursors of NAD biosynthesis. HepG3 cells are cultured in a vitamin B3-deficient RPMI+dialyzed-FBS media for 72 hours. The compounds of Formula V, Formula VII, and/or Formula IX are added to the culture at a concentration equivalent to 6 μM vitamin B3. Cell survival is measured after 24 and 48 hours by CellTiter-Fluor™ viability assay.

Part II: Evaluation of the compounds as direct precursors to the NAD(H) pool. Cells from Part I of the study that can positively rescue the vitamin B3-deficiency are evaluated under the same conditions and in the presence of FK866, an inhibitor of nicotinamide phosphoribosyltransferase (NAMPT). Due to the inhibition of NAMPT, cells only survive if the supplemented compounds of Formula V, Formula VII, and/or Formula IX are capable of directly rescuing the cells' NAD content.

### Example 9. Stability of NRLR-Cl and NRLR-OTf Esters

The solubility of triflate salt forms of NR esters vs. chloride forms in water are very different, i.e., the chloride form of NRLR is soluble in water while the triflate form is not. To ensure that the stability rather than the solubility could be investigated, both the triflate and the chloride form of NRLR (“NRLR-OTf” and “NRLR-Cl”, respectively) were tested for their stability in aqueous buffers and as substrates of the enzyme purine nucleoside phosphorylase (PNP). The release of nicotinamide (NAM) was measured, which is indicative of water-catalyzed hydrolysis. Further, the release of ribose vs. phosphoriboside was monitored, which is indicative of PNP activity. Loss of esters via hydrolysis in buffer catalyzed condition also occurs over time. Once the C5-hydroxyl is free, PNP recognizes the nucleoside and catalyze phosphorolysis. It is not known whether this hydrolysis must occur for all the esters before PNP can act on the nucleoside. The scheme below illustrates the reaction with a partially deprotected nucleoside.

NR triacetate (NRTA) is stable to PNP compared with NR. Thus, NRTA and esters of NRLR-Cl may be suitable precursors to NR in circulation.

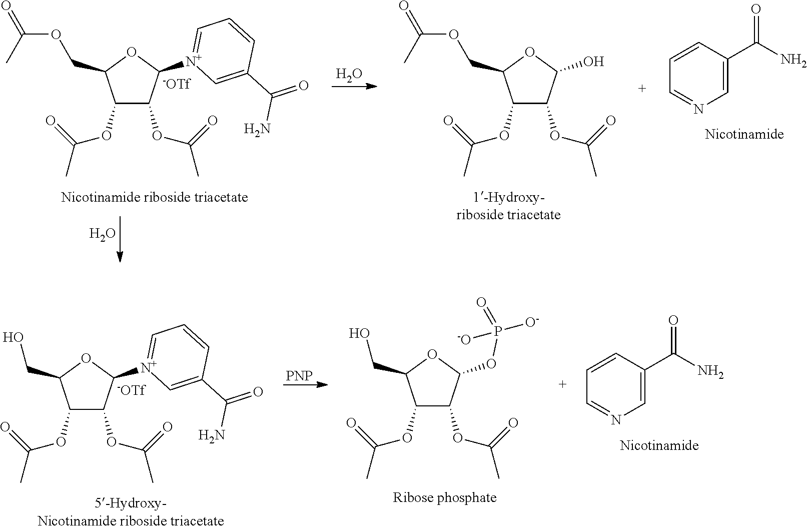

### PNP Enzyme Activity Assay

Phosphorolysis of NRCl by PNP (Sigma Aldrich) was performed in HEPES buffer, containing KH₂PO₄ and 10% D₂O at 25° C. and was monitored by 1H NMR.

Incubations were conducted in the NMR tube which contained a final volume of 505 μl including 450 μl HEPES buffer (100.0 mM, pH 7.0), containing 100 mM KH₂PO₄, 50.0 μl NRCl (100.0 mM in 1 mL D₂O) and when appropriate 5 μl PNP (1 mg dissolved in 50 μl HEPES buffer) was added and measurements were taken at t=5 min and 15 min.

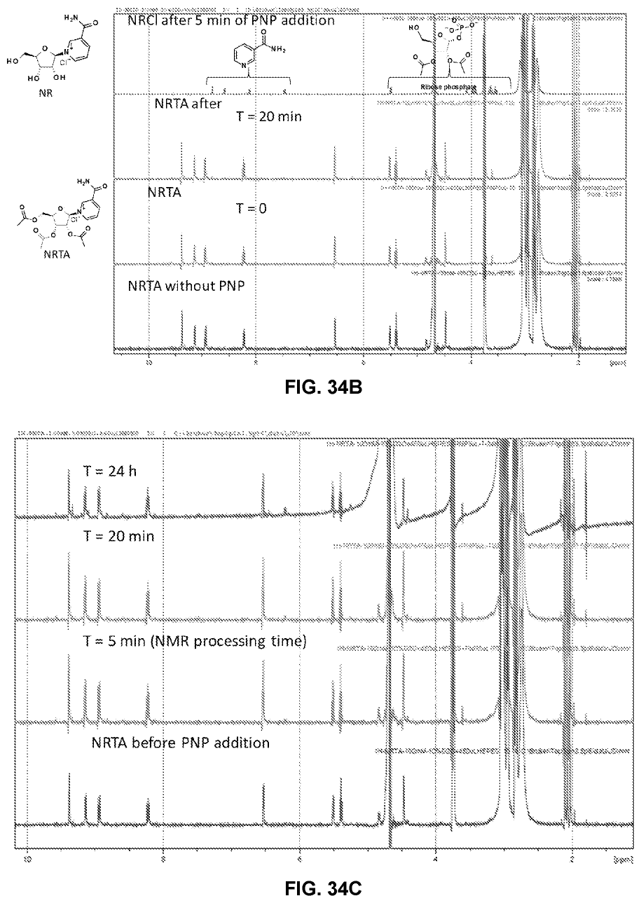

Results are shown in FIGS. 34A, 34B, and 34C. Within 5 min, a complete conversion of NR to NAM occurred, showing that the enzyme was potent to carry out the glycosidic bond cleavage (FIG. 34A and FIG. 34B, top panel). NRTA was found to be stable to PNP, which was attributed to the 5′-acetyl group conferring resistance to PNP activity. (FIG. 34B, 2^{nd} to 4^{th} panels and FIG. 34C). After 24 hours, NRTA is very slowly hydrolyzed and releases NAM. Slow partial ester hydrolysis is also observed as a new set of “NR” peaks emerge. The results demonstrate that, over time, NR triesters slowly release NR by simple chemical hydrolysis (saponification).

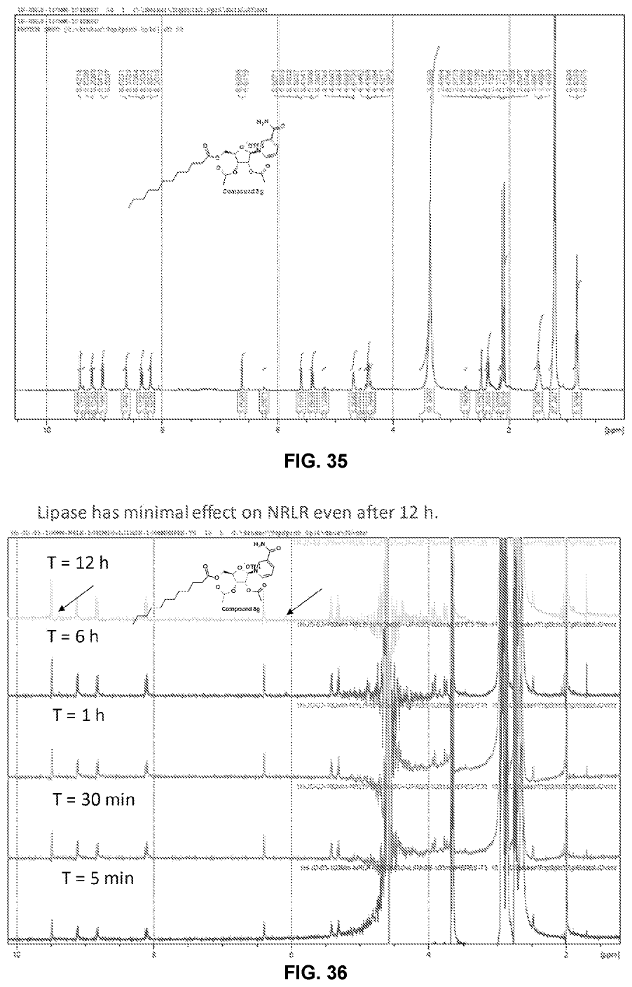

Note: Compound 8g (NRLR triflate) was not completely soluble in water, therefore it was dissolved in DMSO for PNP activity. Similarly, PNP study of NRAD-triflate was conducted in DMSO. The ¹H NMR spectra of NRLR-triflate in 100% DMSO is shown in FIG. 35.

### Lipase Assay

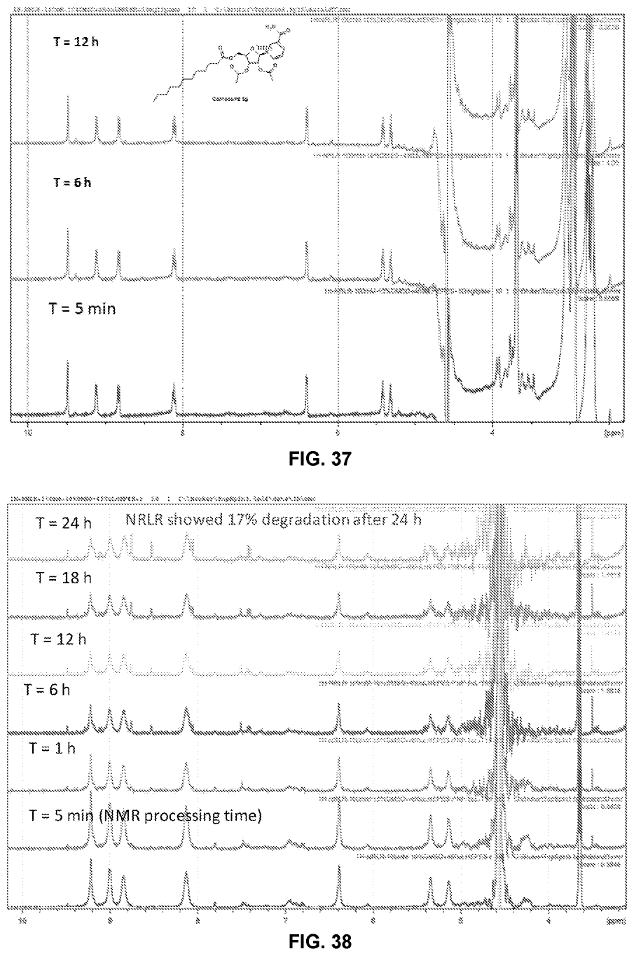

NRLR was subjected to a lipase assay in 10% DMSO, 450 μL HEPES buffer, and enhanced lipase (Candida rugens). Results are shown in FIG. 36, indicating that NRLR remained mostly intact even after 12 hours of incubation with the lipase, with only partial loss of one ester (<5%). FIG. 37 further shows that an increased concentration of lipase had no effect on NRLR over 12 hours of incubation, confirming that NRLR is stable to lipase in HEPES buffer.

Laurate Stability with PNP

The effect of PNP on compound 8g (NRLR) was studied over various time intervals, and the overall increase in NAM was 17% over 24 hours. In addition, partial deprotection of the esters over time is observed, e.g., as indicated by sharper peaks at 9.5 ppm in FIG. 38. This indicates the C5-ester is partially released over time, since the lipidic chain of laurate is likely responsible for the restricted rotation and broadening of the peaks in the NMR spectra.

Dimer Stability with PNP

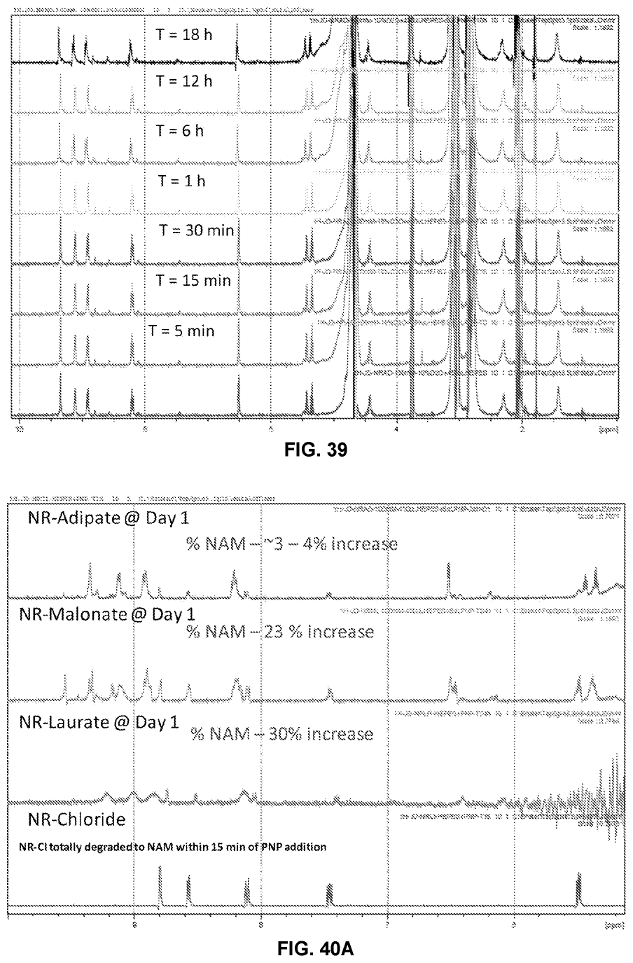

The effect of PNP on compound 8d (NR adipate) was studied over various time intervals, and the overall increase in NAM was 6.3% over 18 hours. NR adipate appears stable to PNP, as shown in FIG. 39. Hydrolysis of the nucleosidic bond was predominant, while the ester dimer appeared to remain intact.

### PNP Activity Assay on NRCl, Compound 8d, Compound 8a, and Compound 8g

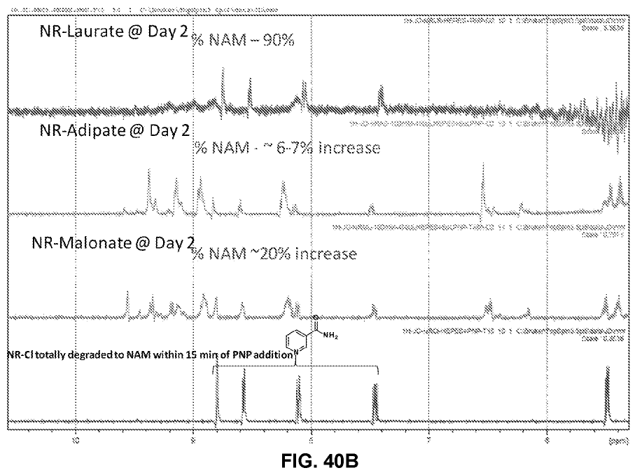

The effect of PNP on NRCl, compound 8d (NR adipate or NRAD), compound 8a (NR malonate or NRML), and compound 8g (NRLR) was compared over 2 days. The results after 1 day are shown in FIG. 40A. NRCl completely degraded to NAM within 15 minutes of PNP addition, while NR adipate, NR malonate, and NRLR respectively had 3-4%, 23%, or 30% increase in NAM after 1 day.

The results after 2 days are shown in FIG. 40B. After 2 days, NRLR (compound 8g) was almost completely degraded (˜90%) to NAM, while NR adipate (compound 8d) and NR malonate still showed some NR diester peaks.

### CONCLUSION

As demonstrated in this Example, NRCl is least stable to PNP enzyme and degrades within few minutes (5-10 min) after PNP addition. PNP degrades NR to NAM and ribose 5′-phosphate. DMSO or HEPES do not affect the enzyme activity either in combination or in mixture. Enzyme activity does not differ in DMSO or D₂O. Lipase does not result in any deacetylation even at higher concentrations or extended period of time. NRTA is more stable to PNP than NRCL. NR laurate (monoester) is less stable compared to NR adipate (diester). NR malonate is less stable compared to NR adipate. In addition to NAM and ribose phosphate, partial deacetylation was witnessed for NR diesters.

The overall trend of stability of NR esters to PNP is as follows:
NRTA>NRAD>NRML>NRLR>NRCl.

### Example 10. Determination of Lipophilicity of NRLR-Cl

The octanol-water partition coefficient (P_{ow}) for NRLR-Cl (compound 10g) was determined. As discussed in Lipinski et al., Adv Drug Deliv Rev 23(1-3):3-25 (1997), the log P_{OW} of a compound intended for oral administration should be less than 5. The predicted log P_{OW} of NRLR-Cl is 2.0284 as determined by BIOVIA DRAW 2019® software.

The experiment was performed as described in “Measuring Lipophilicity with NMR,” SpinSolve Carbon®, MagriTek (2014). Briefly, ¹H NMR spectra were recorded for NRLR-Cl in D₂O, then an equivalent volume of 1-octanol was added to the same NMR tube, and the ¹H NMR was recorded again. The measurements were performed in triplicate. The P_{ow} was calculated based on the concentrations of the NRLR-Cl in the D₂O layer and in the octanol layer, as determined based on the NMR peak values. Results are shown in Table 1. The average measured log P_{OW} of NRLR-Cl is 1.816.

TABLE 1
NRLR-CI Peak Values, Pow, and Log Pow

| | Peak values in | Peak values after | | |
|---|---|---|---|---|
| | water layer | octanol addition | Pow | LogPow |
| Expt - 1 | 0.0478 | 0.0006 | 78.66 | 1.89 |
| Expt - 2 | 0.0386 | 0.0006 | 63.33 | 1.80 |
| Expt - 3 | 0.0539 | 0.0009 | 58.88 | 1.76 |
| Average | | | | 1.816 |
| (LogPow) | | | | |

1. A compound of Formula I:
A-R1-A′ [Formula I]
wherein A and A′ are independently H or 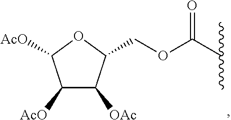
wherein A and A′ are not both H, and:
when one of A or A′ is H, R1 is a substituted or unsubstituted C₈₋₂₄ alkyl, C₈₋₂₄ alkenyl, aryl, alkylaryl, or alkenylaryl; or
when A and A′ are both not H, R1 is a substituted or unsubstituted C₁₋₄ alkyl, C₂₋₄ alkenyl, alkyl polyethylene glycol (PEG) ester, or C₁₋₃ alkyl carboxylate optionally substituted with a protected or free amine; and
Ac is an acetyl group.

2. The compound of claim 1, wherein R1 is an alkyl PEG ester.

3. The compound of claim 2, wherein the PEG ester alkyl comprises 10 to 100 ethylene glycol units.

4. A compound of Formula II: 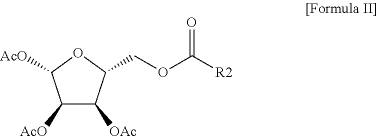
wherein R2 is a substituted or unsubstituted C₈₋₂₄ alkyl, C₈₋₂₄ alkenyl, aryl, alkylaryl, alkenylaryl, or PEG ester alkyl, and
Ac is an acetyl group.

5. The compound of claim 4, wherein R2 is a C₁₂ alkyl, a C₁₈ alkenyl, a C₂₀ alkenyl, or a C₂₂ alkenyl.

6. The compound of claim 5, wherein 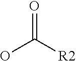
is a substituted or unsubstituted palmitoyl, oleoyl, linoleoyl, linolenoyl, arachidonoyl eicosapentaenoyl, or docosahexaenoyl group.

7. The compound of claim 4, wherein 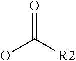
is a succinate ester of retinol.

8. The compound of claim 4, wherein 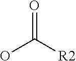
is an omega-3 fatty acid ester.

9. The compound of claim 4, wherein R2 is a substituted or unsubstituted phenyl or benzyl.

10. The compound of claim 9, wherein R2 is a benzyl that comprises an amino substituent.

11. The compound of claim 9, wherein R2 is a para-aminobenzyl.

12. The compound of claim 4, wherein R2 is a mycophenolate.

13. The compound of claim 4, wherein R2 is an alkyl PEG ester.

14. The compound of claim 13, wherein the alkyl PEG ester comprises 10 to 100 ethylene glycol units.

15. A compound of Formula III: 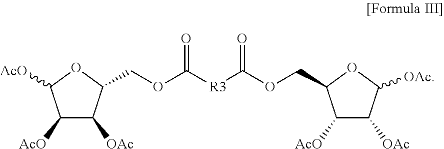
wherein R3 is a substituted or unsubstituted C₁₋₄ alkyl, C₂₋₄ alkenyl, or C₁₋₃ alkyl carboxylate optionally substituted with a protected or a free amine,
or wherein R3 comprises a carbon bonded to (i) a hydroxyl group and (ii) one of 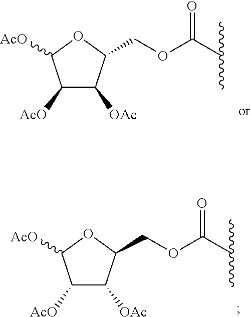 [SMILES: *C(=O)OC[C@H]1OC(OC(C)=O)[C@H](OC(C)=O)[C@@H]1OC(C)=O ; *C(=O)OC[C@@H]1OC(OC(C)=O)[C@@H](OC(C)=O)[C@H]1OC(C)=O]
and
Ac is an acetyl group.

16. The compound of claim 15 wherein the compound of Formula III is any one of 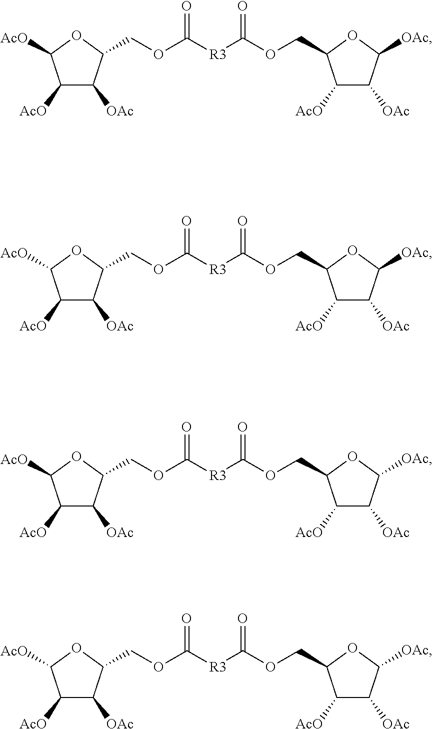
or a mixture thereof.

17. The compound of claim 15 or 16, wherein R3 is an unsubstituted C₁₋₄ alkyl.

18. The compound of claim 15 or 16, wherein R3 is C₂ alkenyl.

19. The compound of claim 15 or 16, wherein R3 is a C₁₋₃ alkyl substituted with a Boc-protected amine.

20. The compound of claim 15 or 16, wherein R3 is a C₁₋₃ alkyl substituted with an N-Boc protected glutamate or aspartate.

21. The compound of claim 15 or 16, wherein R3 is a C₃ alkyl substituted with

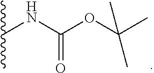 [SMILES: *NC(=O)OC(C)(C)C]

22. The compound of claim 15, wherein the compound of Formula III is:

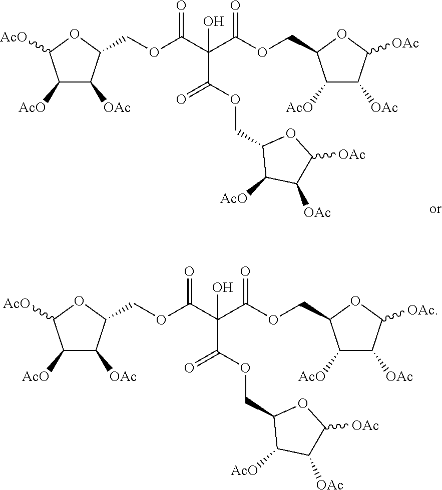 [SMILES: CC(=O)OC1O[C@@H](COC(=O)C(O)(C(=O)OC[C@H]2OC(OC(C)=O)[C@H](OC(C)=O)[C@@H]2OC(C)=O)C(=O)OC[C@H]2OC(OC(C)=O)[C@H](OC(C)=O)[C@@H]2OC(C)=O)[C@H](OC(C)=O)[C@@H]1OC(C)=O ; CC(=O)OC1O[C@H](COC(=O)C(O)(C(=O)OC[C@H]2OC(OC(C)=O)[C@H](OC(C)=O)[C@@H]2OC(C)=O)C(=O)OC[C@H]2OC(OC(C)=O)[C@H](OC(C)=O)[C@@H]2OC(C)=O)[C@@H](OC(C)=O)[C@H]1OC(C)=O]

23. A compound of Formula IV:
B—R1-B′ [Formula IV]
wherein B and B′ are independently H or 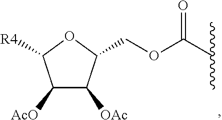
wherein B and B′ are not both H, and:
when one of B or B′ is H, R1 is a substituted or unsubstituted C₈₋₂₄ alkyl, C₈₋₂₄ alkenyl, aryl, alkylaryl, or alkenylaryl; or
when B and B′ are both not H, R1 is a substituted or unsubstituted C₁₋₄ alkyl, C₁₋₄ alkenyl, PEG ester alkyl, or C₁₋₃ alkyl carboxylate optionally substituted with a protected or free amine;
R4 is a nucleobase; and
Ac is an acetyl group.

24. The compound of claim 23, wherein R1 is an alkyl PEG ester.

25. The compound of claim 23, wherein the PEG ester alkyl comprises 10 to 100 ethylene glycol units.

26. A compound of Formula V:
C—R1-C′ [Formula V]
wherein C and C′ are independently H or 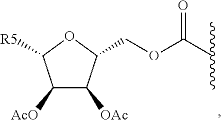
wherein C and C′ are not both H, and:
when one of C or C′ is H, R1 is a substituted or unsubstituted C₈₋₂₄ alkyl, C₈₋₂₄ alkenyl, aryl, alkylaryl, or alkenylaryl; or
when C and C′ are both not H, R1 is a substituted or unsubstituted C₁₋₄ alkyl, C₁₋₄ alkenyl, PEG ester alkyl, or C₁₋₃ alkyl carboxylate optionally substituted with a protected or free amine;
R5 is nicotinamide, dihydronicotinamide, nicotinic acid, nicotinic acid ester, or a reduced form thereof, and
Ac is an acetyl group.

27. The compound of claim 26, wherein R1 is an alkyl PEG ester.

28. The compound of claim 26, wherein the PEG ester alkyl comprises 10 to 100 ethylene glycol units.

29. A compound of Formula VI: 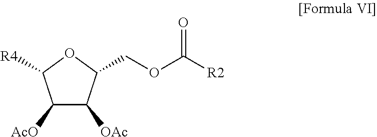
wherein R2 is a substituted or unsubstituted C₈₋₂₄ alkyl, C₈₋₂₄ alkenyl, aryl, alkylaryl, alkenylaryl, or PEG ester alkyl;
R4 is a nucleobase; and
Ac is an acetyl group.

30. The compound of claim 29, wherein R2 is a C₁₂ alkyl, a C₁₈ alkenyl, a C₂₀ alkenyl, or a C₂₂ alkenyl.

31. The compound of claim 30, wherein 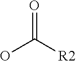
is a substituted or unsubstituted palmitoyl, oleoyl, linoleoyl, linolenoyl, arachidonoyl eicosapentaenoyl, or docosahexaenoyl group.

32. The compound of claim 29, wherein 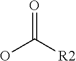
is a succinate ester of retinol.

33. The compound of claim 29, wherein 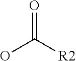
is an omega-3 fatty acid ester.

34. The compound of claim 29, wherein R2 is a substituted or unsubstituted phenyl or benzyl.

35. The compound of claim 34, wherein R2 is a benzyl that comprises an amino substituent.

36. The compound of claim 34, wherein R2 is a para-aminobenzyl.

37. The compound of claim 29, wherein R2 is a mycophenolate.

38. The compound of claim 29, wherein R2 is an alkyl PEG ester.

39. The compound of claim 38, wherein the alkyl PEG ester comprises 10 to 100 ethylene glycol units.

40. A compound of Formula VII: 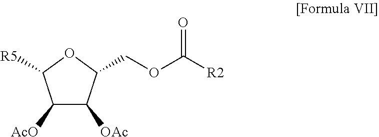
wherein R2 is a substituted or unsubstituted C₈₋₂₄ alkyl, C₈₋₂₄ alkenyl, aryl, alkylaryl, alkenylaryl, or PEG ester alkyl;
R5 is nicotinamide, dihydronicotinamide, nicotinic acid, nicotinic acid ester, or a reduced form thereof; and
Ac is an acetyl group.

41. The compound of claim 40, wherein R2 is a C₁₂ alkyl, a C₁₈ alkenyl, a C₂₀ alkenyl, or a C₂₂ alkenyl.

42. The compound of claim 41, wherein 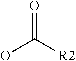
is a substituted or unsubstituted palmitoyl, oleoyl, linoleoyl, linolenoyl, arachidonoyl eicosapentaenoyl, or docosahexaenoyl group.

43. The compound of claim 40, wherein 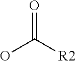
is a succinate ester of retinol.

44. The compound of claim 40, wherein 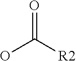
is an omega-3 fatty acid ester.

45. The compound of claim 40, wherein R2 is a substituted or unsubstituted phenyl or benzyl.

46. The compound of claim 45, wherein R2 is a benzyl that comprises an amino substituent.

47. The compound of claim 45, wherein R2 is a para-aminobenzyl.

48. The compound of claim 40, wherein R2 is a mycophenolate.

49. The compound of claim 40, wherein R2 is an alkyl PEG ester.

50. The compound of claim 49, wherein the alkyl PEG ester comprises 10 to 100 ethylene glycol units.

51. A compound of Formula VIII: 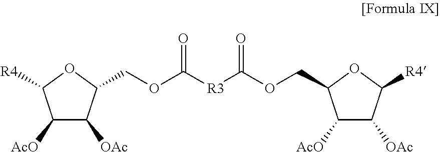
wherein R3 is a substituted or unsubstituted C₁₋₄ alkyl, C₁₋₄ alkenyl, or C₁₋₃ alkyl optionally substituted with a protected or free amine,
or wherein R3 comprises a carbon bonded to (i) a hydroxyl group and (ii) one of 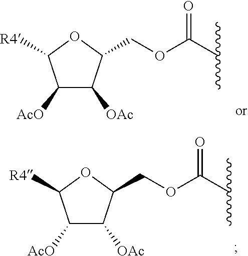
R4, R4′, and R4″ are each independently a nucleobase; and
Ac is an acetyl group.

52. The compound of claim 51, wherein R3 is an unsubstituted C₁₋₄ alkyl.

53. The compound of claim 51, wherein R3 is C₂ alkenyl.

54. The compound of claim 51, wherein R3 is a C₁₋₃ alkyl substituted with a Boc-protected amine.

55. The compound of claim 51, wherein R3 is a C₁₋₃ alkyl substituted with an N-Boc protected glutamate or aspartate.

56. The compound of claim 51, wherein R3 is a C₃ alkyl substituted with

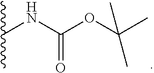 [SMILES: *NC(=O)OC(C)(C)C]

57. The compound of claim 51, wherein the compound of Formula VIII is

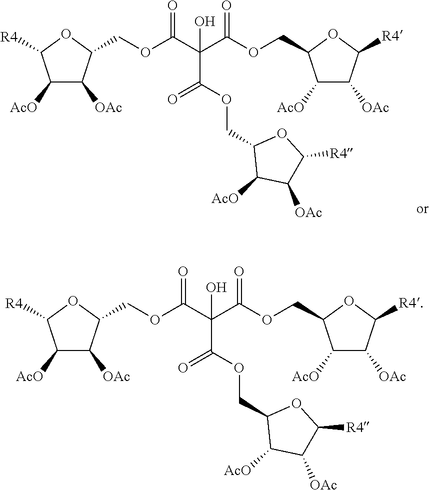

58. A compound of Formula IX: 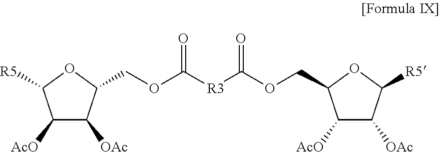
wherein R3 is a substituted or unsubstituted C₁₋₄ alkyl, C₁₋₄ alkenyl, or C₁₋₃ alkyl optionally substituted with a protected or free amine,
or wherein R3 is a carbon bonded to (i) a hydroxyl group and (ii) one of 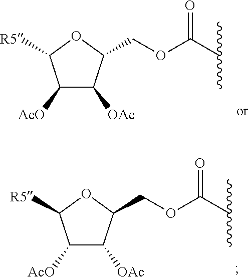
wherein R5, R5′ and R5″ are each independently nicotinamide, dihydronicotinamide, nicotinic acid, nicotinic acid ester, or a reduced form thereof; and
Ac is an acetyl group.

59. The compound of claim 58, wherein R3 is an unsubstituted C₁₋₄ alkyl.

60. The compound of claim 58, wherein R3 is C₂ alkenyl.

61. The compound of claim 58, wherein R3 is a C₁₋₃ alkyl substituted with a Boc-protected amine.

62. The compound of claim 58, wherein R3 is a C₁₋₃ alkyl substituted with an N-Boc protected glutamate or aspartate.

63. The compound of claim 58, wherein R3 is a C₃ alkyl substituted with

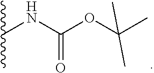 [SMILES: *NC(=O)OC(C)(C)C]

64. The compound of claim 58, wherein the compound of Formula IX is:

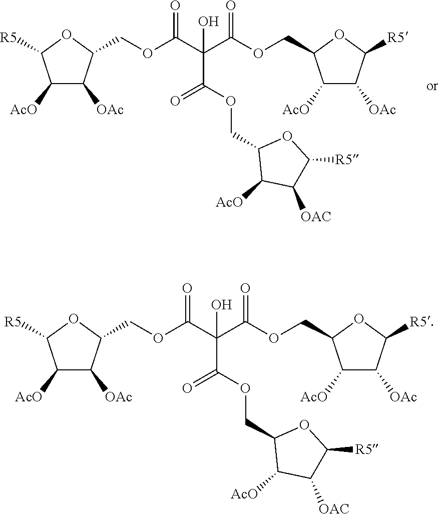

65. A method of making a compound of Formula I, Formula II, or Formula III, comprising:
(a) adding a protecting group to the 5′-carbon of D-ribose, to form a 5′-protected ribose;
(b) acetylating the hydroxy groups at the 1′, 2′, and 3′ carbons of the 5′-protected ribose, to form an acetylated, 5′-protected ribose;
(c) deprotecting the 5′-carbon of the acetylated, 5′-protected ribose, to form an acetylated, 5′-deprotected ribose; and
(d) coupling the acetylated, 5′-deprotected ribose with a reactant comprising R1, R2, and/or R3, to form the compound of Formula I, Formula II, or Formula III.

66. The method of claim 65, wherein the coupling is performed by a mechanochemical reaction.

67. The method of claim 65 or 66, wherein the reactant of (d) comprises an acid chloride, an acid anhydride, a dicarboxylic acid, or a monocarboxylic acid of R1, R2, and/or R3.

68. A method of making a compound of Formula IV, Formula V, or Formula VI, comprising reacting a compound of Formula I, Formula II, or Formula III with a functionalized nucleobase to form the compound of Formula IV, Formula V, or Formula VI.

69. A method of making a compound of Formula VII, Formula VIII, or Formula IX, comprising reacting a compound of Formula I, Formula II, or Formula III with a functionalized nicotinamide to form the compound of Formula IV, Formula V, or Formula VI.

70. The method of claim 68 or 69, wherein the reacting is performed by a mechanochemical reaction.

71. A composition comprising the compound of any one of claims 1 to 64 and a pharmaceutically acceptable excipient.

72. A method of treating nicotinamide adenine dinucleotide (NAD) deficiency in a subject in need thereof, comprising administering the compound of any one of claims 26 to 28, any one of claims 40 to 50, or any one of claims 58 to 64 to the subject.

73. A method of increasing nicotinamide adenine dinucleotide (NAD) in a subject in need thereof, comprising administering the compound of any one of claims 26 to 28, any one of claims 40 to 50, or any one of claims 58 to 64 to the subject.

74. The method of claim 72 or 73, further comprising administering pterostilbene to the subject.
